(19) United States
(12) Patent Application Publication
(10) Pub. No.: US 2025/0011869 A1
(43) Pub. Date: Jan. 9, 2025

(54) TLR3 INHIBITORS FOR USE IN THE TREATMENT OF DISEASES ASSOCIATED WITH CARDIOVASCULAR CALCIFICATION

(71) Applicant: Johannes HOLFELD, Innsbruck (AT)

(72) Inventors: Johannes HOLFELD, Innsbruck (AT); Can GOLLMANN-TEPEKOYLU, Sistrans (AT); Ivan TANCEVSKI, Hall i, Tirol (AT)

(21) Appl. No.: 18/702,233

(22) PCT Filed: Oct. 19, 2022
(86) PCT No.: PCT/EP2022/079004

§ 371 Date: Apr. 17, 2024

(30) Foreign Application Priority Data
Oct. 19, 2021 (EP) 21 203 501.8

(51) Int. Cl. C12Q 1/6883; A61K 31/135; A61K 31/343; A61K 31/381; A61K 31/4706; A61K 31/473; A61K 31/495; A61K 31/5415

(52) U.S. Cl. CPC C12Q 1/6883; A61K 31/135; A61K 31/343; A61K 31/381; A61K 31/4706; A61K 31/473; A61K 31/495; A61K 31/5415; C12Q 2600/156

(57) ABSTRACT

The present invention generally relates to products, compositions and methods for the diagnosis, prognosis, prophylaxis and treatment of diseases associated with cardiovascular calcification. The invention provides an inhibitor of toll-like receptor 3 (TLR3) for use in the treatment of a disease associated with cardiovascular calcification. Furthermore, the invention relates to a method for assessing whether a subject is at increased risk of developing a disease associated with cardiovascular calcification. The invention also provides a substance for use in a method of in vivo diagnosis of a disease associated with cardiovascular calcification in a subject.

The present invention generally relates to products, compositions and methods for the diagnosis, prognosis, prophylaxis and treatment of diseases associated with cardiovascular calcification.

The invention provides an inhibitor of toll-like receptor 3 (TLR3) for use in the treatment of a disease associated with cardiovascular calcification.

Furthermore, the invention relates to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present.

The invention also provides a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

Calcific aortic valve disease (CAVD) is the most common type of valvular disease; it affects over 5.2 million Americans and its prevalence expected to increase due to ageing society (Danielsen et al., 2014). Age represents the most prominent risk factor for CAVD (Eveborn et al., 2013), characterized by dysregulated innate immunity, a phenomenon termed inflammaging (Franceschi et al., 2018).

During its lifespan, the aortic valve is subjected to excessive mechanical strain and cellular injury, which in turn is known to promote the formation of calcific lesions (Back et al., 2013). Endogenous danger signals are released upon cellular injury, called danger associated molecular patterns (DAMPs), which may initiate and sustain aortic valve calcification. Toll-like receptors (TLRs) are evolutionary highly conserved pattern recognition receptors that orchestrate the inflammatory response to pathogens and are activated by pathogen associated molecular patterns (PAMPs) (Kawai and Akira, 2010). Because ligand selectivity is relatively low, endogenous DAMPs can also activate TLRs. TLR activation induces inflammation, contributing to cardiovascular disease including atherosclerosis (Kiechl et al., 2002), heart failure (Frantz et al., 1999) and valvular heart disease (Garcia-Rodriguez et al., 2018; Yang et al., 2009). There is clear evidence that TLRs are involved in the pathogenesis of CAVD. In particular TLR2 and TLR4 activated by bacterial PAMPs were shown to contribute to CAVD via Notch1 and NFKB-mediated inflammation (Yang et al., 2009; Zeng et al., 2013). Hence, the evolutionary advantage of improved defense against infections might come at the cost of increased risk for cardiovascular disease, representing a model of pleiotropic antagonism: a receptor crucial in the inflammatory host response may become detrimental at later age (Wagner and Zhang, 2011).

TLR3 stands out amongst all other TLRs. In contrast to all other TLRs, its signaling is not dependent on Myeloid differentiation primary response 88 (MyD88) but on TIR-domain-containing adapter-inducing interferon-β (TRIF). TLR3 activation results in interferon regulatory factor (IRF3)-dependent transcription of type I interferons (Kawai and Akira, 2010). Of note, type I interferons are not only implicated in the host's response to viral pathogens, but are also involved in physiological calcification and bone formation. In this regard, Ifnb^{-/-} and Ifnar1^{-/-} mice deficient in type I interferons exhibit a distinct phenotype of impaired bone formation and osteoporosis (Li, 2013; Takayanagi et al., 2002). Moreover, mutations in the pattern recognition receptor melanoma differentiation antigen 5 (MDA5) that result in aberrant IFN signaling cause pathological vascular and valvular calcifications with osteopenia in children with Singleton-Merton syndrome (Rutsch et al., 2015). TLR3, located in endosomes, recognizes viral dsRNA as well as endogenous RNA released from injured cells, acting as sensor of tissue injury initiating inflammation (Cavassani et al., 2008; Blasius and Beutler, 2010). Activation of TLR3 in aortic valvular interstitial cells (VICs) can trigger osteogenic responses (Zhan et al., 2015), but the physiological relevance of this finding is incompletely understood.

VICs are the predominant cell type within the aortic valve and are responsible for the maintenance of valvular function. Importantly, a phenotypic switch of VICs to bone-forming osteoblasts leads to progressive aortic valve calcification (Liu et al., 2007). However, the trigger for this phenotype switch is unknown. This lack of mechanistic insight stymies the development of effective intervention strategies for CAVD. Replacement of the aortic valve via invasive surgery can prevent valvular cardiomyopathy and death, but the complications and risk associated with surgery limit its practical utility. Currently, there are no pharmacological treatment options to stop CAVD progression. Oxidized low-density lipoproteins (OxLDL) and OxLDL-derived phospholipids promote CAVD and GWAS revealed that genetic variation in Lp(a) constitutes a risk factor for CAVD. Nevertheless, statins, the most widely used hypolipidemic drug class in the clinic, were not effective against CAVD.

Thus, there is a need for means and methods for (early) diagnosis, prophylaxis and treatment of diseases associated with cardiovascular calcification such as calcific aortic valve disease (CAVD) and/or coronary heart disease (CHD). Thus, the technical problem underlying the present invention is the provision of means and methods for (early) diagnosis, prophylaxis and treatment of diseases associated with cardiovascular calcification such as calcific aortic valve disease (CAVD) and/or coronary heart disease (CHD).

The technical problem is solved and/or the above-mentioned needs are addressed by the provision of the embodiments characterized in the claims and as provided herein below.

The invention provides an inhibitor of toll-like receptor 3 (TLR3) for use in the treatment of a disease associated with cardiovascular calcification.

The invention also provides a method of treating a disease associated with cardiovascular calcification, the method comprising administering an inhibitor of TLR3.

Furthermore, the invention relates to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present.

Furthermore, the invention relates to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

In addition, the invention relates to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The present invention solves the above identified technical problem since, as documented herein below and in the appended examples, it was surprisingly found that Tlr3^{-/-} mice are protected from CAVD. Tlr3^{-/-} mice, i.e. mice that lack the biological function of a TLR3 do not show any signs of thickening of the aortic valve leaflets and calcification usually developing with advanced age. Further, the present invention surprisingly showed that ApoE^{-/-}; Tlr3^{-/-} double-knockout mice were protected from hyperlipidemia-induced CAVD but did not show differences in serum cholesterol, tryglyceride levels or weight, therefore presenting sound evidence that TLR3 is directly involved in the induction of cardiovascular calcification which leads to conditions such as CAVD and/or CHD.

The present invention surprisingly showed that TLR3 mediated induction of calcification is further promoted by application of mechanical stress onto cells. Such mechanical stress is present in the cardiovascular system e.g. in the form of hemodynamic stress which can lead to cellular injury.

Further, it was surprisingly demonstrated that activation of TLR3, e.g. by binding of a ligand, promotes a pathological phenotype switch, i.e. the transdifferentiation of valvular interstitial cells into osteoblast-like cells upon exposure to mechanical strain. The onset of this osteoblast-related pathway initiated by TLR3 activation promotes cardiovascular calcification.

Taken together the current invention presents evidence that an inhibitor of TLR3 and/or of the TLR3 pathway including upstream and downstream pathway components, such as ligands, would be able to reduce the effect of TLR3 in the onset of calcification processes.

The present invention unexpectedly identified such a ligand of TLR3 as the proteoglycan biglycan (BGN), a structural protein of the extracellular matrix (ECM) that induces TLR3 expression in a dose-dependent manner by directly interacting with TLR3. Another unexpected result was that administration of a TLR3 inhibitor resulted in reduced calcification in a VICs cell culture model and in zebrafish in vivo.

Another surprising finding was that GWA meta-analysis of two large-scale cohorts with >300,000 individuals of European ancestry revealed that genetic variations at loci relevant to the TLR3 pathway are associated with clinically relevant aortic valve calcification. Thus, the present invention solves the above stated technical problem by provision of an early risk assessment of a disease associated with cardiovascular calcification, such as CAVD and/or CHD in subjects/patients. Prevention of said diseases may be realized by a prophylactic treatment with a drug(s) available in the art and/or with a TLR3 inhibitor in such subjects/patients assessed to be at increased risk of developing said disease(s).

The present invention links bone development with CAVD pathogenesis and uncovered TLR3 as a therapeutic target for use in the treatment or prevention of a disease associated with cardiovascular calcification. The present invention thus solves the above mentioned technical problem by the provision of an inhibitor, that can prevent interaction and activation of TLR3, e.g. by a ligand such as biglycan.

In the prior art, TLR3 inhibition was successfully achieved in experimental settings via small molecule inhibitors or blocking antibodies^{1,2}. TLR3 neutralizing antibodies have already been tested in patients with asthma or colitis³. Although none of the trials proved efficacy, inhibition of TLR3 met safety criteria, which may allow for a straight-forward drug repurposing strategy to tackle CAVD in the setting of clinical trials.

In the prior art it was further shown that TLR2 and TLR4 activated by bacterial PAMPs contribute to CAVD via Notch1 and NFKB-mediated inflammation^{4,5}. However, to target TLR3 is not an obvious choice. There are more than 10 different TLRs described with all of them showing different function and signaling. The present invention provides for the first time a very specific pathway and links TLR3 signaling to interferon-mediated calcification. TLR2 and TLR4 signaling do not result in interferon-signaling, but mainly in NFKB-mediated inflammatory response.

Further it was shown that stimulation of TLR3 by an agonist results in an unspecific inflammatory response with upregulation of TGF-ß and IL-1b and two calcific factors (BMP2, ALP) in vitro. However, TLR3 stands out amongst all other TLRs. In contrast to all other TLRs, its signaling is not dependent on Myeloid differentiation primary response 88 (MyD88) but on TIR-domain-containing adapter-inducing interferon-β (TRIF). Activation can result either in interferon regulatory factor (IRF3)-dependent transcription of type I interferons, or in NFKB-mediated transcription of proinflammatory cytokines including IL-6 and TNF-a. The NFKB response can be induced by other TLRs as well (e.g. TLR2 and TLR4), IRF3 is specific for nucleic acid TLRs. Zhan et al. 2015 disclose that TLR3 mainly induces calcification via NFKB and subsequent phosphorylation of ERK1/2. However, NFKB and ERK signaling is quite unspecific, as it can be activated by many different pathways. In contrast, IRF3 dependent signaling with subsequent regulation of interferons is quite specific. The transcription factor IRF3 translocates into the nucleus and induces the transcription of type I interferons. The present invention shows for the first time that the TLR3-dependent transcription of type I interferons is clearly the cause for calcification in CAVD, rather than the unspecific inflammatory response upon phosphorylation of NFKB. Further, the present invention provides clear evidence for the downstream pathway involved in CAVD, which includes IRF3, IFNAR1, JAK/STAT and Runx2, including functional animal data.

Until now the treatment of diseases associated with cardiovascular calcification was limited to invasive repair and/or surgery at a late stage of the disease. However, the complications and risk associated with surgery limit its practical utility.

The present invention provides for the first time means and methods to prevent diseases associated with cardiovascular calcification such as CAVD and/or CHD. Thus, while the present invention generally relates to the treatment of diseases associated with cardiovascular calcification, the treatment preferably is the prophylaxis (prevention) of such diseases, such as CAVD and/or CHD.

As explained elsewhere herein, the term “cardiovascular calcification” means ectopic buildup of mineral deposits, such as calcium minerals, that form plaques in cardiovascular tissues. Coronary heart disease, or CHD, is one of the most common heart diseases in industrialized countries. It is associated with one or more constricted coronary vessels, leading to the heart muscles' circulatory disorders and, therefore, to an undersupply of oxygen to the heart. As a result, the heart can no longer work properly, leading to heart pain and tightness in the chest. In the worst case, CHD can lead to a heart attack.

Calcific aortic valve disease (CAVD) is a slow, progressive disorder that ranges from mild valve thickening without obstruction of blood flow, termed aortic sclerosis, to severe calcification with impaired leaflet motion, termed aortic stenosis.

Preferably, the herein provided treatment, particularly with TLR3 inhibitors, is indicated for patients before an irreversible damage manifests in the patient, e.g. a damage (like stenosis) that can only be treated by repair and/or surgery.

Therefore, the herein provided treatment, particularly with TLR3 inhibitors, is in particular indicated for patients that are diagnosed/assessed as being at increased risk of developing or suffering from a disease associated with cardiovascular calcification and/or that do not yet show clinical symptoms, particularly that do not yet show an irreversible damage, e.g. a damage (like stenosis) that can only be treated by repair and/or surgery. In other words, the herein provided treatment, particularly with TLR3 inhibitors, is preferably indicated for patients at an early stage of the disease associated with cardiovascular calcification, such as CAVD and/or CHD, e.g. a stage with (aortic) sclerosis (particularly in CAVD) or even earlier stages, but not at a stage with aortic stenosis.

Likewise, the herein provided methods for diagnosis/risk assessment are particularly useful (and hence preferred) for a disease associated with cardiovascular calcification in subjects, wherein said disease is characterized in that the subjects do not yet show clinical symptoms, particularly that do not yet show an irreversible damage, e.g. a damage (like stenosis) that can only be treated by repair and/or surgery. In other words, the herein provided methods for diagnosis/risk assessment are preferred (to confirm/determine presence of/risk for) a disease associated with cardiovascular calcification in subjects, wherein said disease is at an early stage, such as an early stage of CAVD and/or of CHD, e.g. a stage with (aortic) sclerosis (particularly in CAVD) or even earlier stages, but not at a stage with aortic stenosis. Likewise, the described methods for diagnosis/risk assessment are preferably applied for subjects that are suspected of suffering from (or prone to suffering from) a disease associated with cardiovascular calcification, wherein said disease is at an early stage, such as an early stage of CAVD and/or of CHD, e.g. a stage with (aortic) sclerosis (particularly in CAVD) or even earlier stages, but not at a stage with aortic stenosis.

Cardiovascular calcification may be induced and/or caused and/or promoted by atherosclerosis and/or factors promoting atherosclerosis such as mechanical stress, irradiation, chronic kidney disease, diabetes mellitus, dyslipidemia, hypertension, gender, advanced age, unhealthy diets, sedentary lifestyle, white race/ethnicity, body mass index, family history of diseases associated with cardiovascular calcification, total cholesterol level, high density lipoprotein cholesterol level, consumption of tobacco products and/or others. Cardiovascular calcification may be caused by mechanical stress in the cardiovascular system. Cardiovascular calcification may also be caused by hemodynamic stress. Cardiovascular calcification may further be caused by a combination of hemodynamic stress and factors promoting atherosclerosis. In addition, mechanical stress or other sources of stress/strain may lead to cellular injury, which in turn may cause and/or promote cardiovascular calcification. Cellular injury may be caused e.g. by mechanical stress such as hemodynamic stress.

Accordingly, the herein provided invention is useful and applicable to subjects with the above risk factors. Also drugs which are used in the art to treat patients with such risk factors may be used herein (i.e. patients with an (increased) risk of developing a disease associated with cardiovascular calcification, e.g. patients suffering from hypertension, irradiation (damage), chronic kidney disease, diabetes mellitus, dyslipidemia and/or consumption of tobacco products, and the like). Such drugs are described further hereinbelow and may be used alone or in co-therapy (i.e. one of these drugs combined with one or more of the other drugs) and/or e.g. in combination with a TLR3 inhibitor. Such drugs (alone or in co-therapy as described above) may also be used in the treatment of subjects which are diagnosed to suffer from or diagnosed to be prone to suffering from (i.e. to be at risk of suffering from) a disease associated with cardiovascular calcification by the herein described method of in vivo diagnosis of a disease associated with cardiovascular calcification.

It is also envisaged herein that the means and methods provided by the invention delay the progression of diseases associated with cardiovascular calcification such as CAVD and/or CHD. It is also envisaged herein that the means and methods provided by the invention completely halt/stop the progression of diseases associated with cardiovascular calcification such as CAVD and/or CHD. It is also envisaged herein that the means and methods provided by the invention delay the progression of diseases associated with cardiovascular calcification such as CAVD and/or CHD in a way that the individual suffering from said disease is not affected by any symptoms usually associated with said diseases.

In summary it is illustrated by the present invention that diseases associated with cardiovascular calcification can be treated by a TLR3 inhibitor.

The present invention relates to products, compositions and methods for the diagnosis, risk assessment, prophylaxis (prevention/preventive treatment) and treatment (therapeutic treatment) of diseases associated with cardiovascular calcification. In particular, the present invention provides an inhibitor of TLR3 in the treatment of a disease associated with cardiovascular calcification. The present invention provides for the first time an inhibitor for use in a prophylaxis and/or treatment of diseases associated with cardiovascular calcification such as CAVD and/or CHD. In particular, the method for an early risk assessment of the invention causatively links variants in genomic sequences in or near a gene of a patient with a potential risk of contracting a disease associated with cardiovascular calcification such as CAVD. The present invention further provides a method for an early risk assessment and a substance and method for an early diagnosis of a disease associated with cardiovascular calcification. In particular, the invention provides a method and a substance for use in an early diagnosis of a disease associated with cardiovascular calcification in a subject by measuring the level of accumulation of a radioactively labelled substance that binds TLR3 and visualizing the same in cardiovascular tissue. Further, the invention provides uses to carry out the methods of the present invention and kits comprising components useful to carry out any of the methods, uses and purpose-limited uses of the present invention.

In the following the invention is described in more detail.

In particular the invention relates to the following items:

1. An inhibitor of toll-like receptor 3 (TLR3) for use in the treatment of a disease associated with cardiovascular calcification.

2. A method of treating a disease associated with cardiovascular calcification, the method comprising administering an inhibitor of toll-like receptor 3 (TLR3).

3. The inhibitor for use of item 1, or the method of item 2, wherein the disease is associated with calcification of the aortic valve and/or calcification of the coronary arteries and/or calcification of the great arteries
preferably, wherein the disease is associated with calcification of the aortic valve.

4. The inhibitor for use of item 1 or 3, or the method of item 2 or 3, wherein the disease is calcific aortic valve disease (CAVD) and/or coronary heart disease (CHD),
preferably, wherein the disease is CAVD.

5. The inhibitor for use of item 4, or the method of item 4, wherein the CAVD manifests in aortic sclerosis and/or aortic stenosis.

6. The inhibitor for use of any one of items 1, 3 to 5, or the method of any one of items 2 to 5, wherein the cardiovascular calcification is induced by mechanical stress, irradiation, chronic kidney disease, factors promoting atherosclerosis, genetic predisposition or a combination thereof,
preferably, wherein the cardiovascular calcification is induced by a combination of mechanical stress or factors promoting atherosclerosis,
further preferably, wherein the cardiovascular calcification is induced by mechanical stress,
further preferably, wherein the mechanical stress is hemodynamic stress.

7. The inhibitor for use of any one of items 1, 3 to 6, or the method of any one of items 2 to 6, wherein the inhibitor is one or more of (R)-2-(3-Chloro-6-fluorobenzo [b] thiophene-2-carboxamido)-3-phenyl-propanoic acid], Levocetirizine, ORF 1329L, Sertraline, Fluphenazine, ZL0420, ZL0454, Quinacrine, Chloroquine and Amiodarone.

8. A method for assessing whether a subject is at increased risk of developing a disease associated with cardiovascular calcification, said method comprising,
(i) determining in a sample from said subject whether one or more genomic sequences in or near one or more gene comprises one or more genetic variant; and
(ii) assessing that said subject is at increased risk of developing a disease associated with cardiovascular calcification when the one or more genetic variant in the one or more genomic sequences in or near said one or more gene is present.

9. The method of item 8, wherein the one or more gene is one or more of JAK1, TLR3, IFNB1, IFNA1, XYLT1 or IFNAR1.

10. The method of item 8 or 9, wherein the risk of developing a disease associated with cardiovascular calcification is increased at least 1.5-fold.

11. The method of any one of items 8 to 10, wherein the genetic variant is a single-nucleotide polymorphism (SNP).

12. The method of any one of items 8 to 11, wherein the genetic variant is one or more of
(a) a genomic sequence in or near the human JAK1 gene comprising one or more of:
   (i) variant rs143732508 at position 1:65335640 comprising the nucleobase G;
   (ii) variant rs564691204 at position 1:65342993 comprising the nucleobase T;
   (iii) variant rs528952911 at position 1:65347527 comprising the nucleobase C; and/or
   (iv) variant rs146653955 at position 1:65380580 comprising the nucleobase C;
(b) a genomic sequence in or near the human TLR3 gene comprising one or more of:
   (i) variant rs548870644 at position 4:186953463 comprising the nucleobase G; and/or
   (ii) variant rs184106700 at position 4:187028029 comprising the nucleobase G;
(c) a genomic sequence in or near the human IFNB1 gene and comprises one or more of:
   (i) variant rs569915578 at position 9:21119979 comprising the nucleobase T;
   (ii) variant rs755535058 at position 9:21120058 comprising the nucleobase T; and/or
(d) a genomic sequence in or near the human IFNA1 gene comprising the variant rs551992948 at position 9:21457591 comprising the nucleobase C;
(e) a genomic sequence in or near the human XYLT1 gene comprising one or more of:
   (i) variant rs118001479 at position 16:17153381 comprising the nucleobase A;
   (ii) variant rs550834189 at position 16:17283730 comprising the nucleobase A;
   (iii) variant rs531295111 at position 16:17289368 comprising the nucleobase C;
   (iv) variant rs62033189 at position 16:17342509 comprising the nucleobase C;
   (v) variant rs34588333 at position 16:17345488 comprising the nucleobase A; and/or
   (vi) variant rs936346 at position 16:17376126 comprising the nucleobase C;
(f) a genomic sequence in or near the human IFNAR1 gene comprising the variant rs554831417 at position 21:34683984 comprising the nucleobase T.

13. The method of any one of items 8 to 12, wherein the disease is associated with calcification of the aortic valve and/or calcification of the coronary arteries and/or calcification of the great arteries
preferably, wherein the disease is associated with calcification of the aortic valve.

14. The method of any one of items 8 to 13, wherein the disease is calcific aortic valve disease (CAVD) and/or coronary heart disease (CHD),
preferably, wherein the disease is CAVD.

15. The method of any one of items 8 to 14, wherein the cardiovascular calcification is induced by mechanical stress, irradiation, chronic kidney disease, factors promoting atherosclerosis or a combination thereof
preferably, wherein the cardiovascular calcification is induced by a combination of mechanical stress and factors promoting atherosclerosis,
further preferably, wherein the cardiovascular calcification is induced by mechanical stress,
further preferably, wherein the mechanical stress is hemodynamic stress.

16. Use of a binding molecule, a nucleic acid, a nucleic acid probe, a primer, a primer pair, a biotinylated primer, a SNP microarray, a single-stranded oligonucleotide probe specific for one or more genetic variant of the one or more genomic sequences in or near a gene as defined in any one of items 8 to 15 for assessing whether a subject is at increased risk of developing a disease associated with cardiovascular calcification.

17. A drug for use in the treatment of a subject assessed to be at increased risk of developing a disease associated with cardiovascular calcification according to any one of items 8 to 16.

18. The drug of item 17, wherein the drug comprises an inhibitor of toll-like receptor 3 (TLR3).

19. A substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

20. A method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

21. The substance for use of item 19, or the method of item 20, wherein the substance is radioactively labelled and binds TLR3,
preferably, wherein the substance comprises a TLR3 specific ligand, antibody or nucleic acid probe.

22. The substance for use of item 19 or 21, or the method of item 20 or 21, wherein the substance is administered to the subject intravenously and an accumulation of said substance is detected by positron emission tomography (PET).

23. The substance for use of any one of items 19 to 22, or the method of item 20 to 22, wherein the cardiac tissue is the aortic valve and the accumulation is indicative for the subject being at increased risk of developing or suffering from CAVD, and/or
wherein the cardiac tissue is the coronary arteries and the accumulation is indicative for the subject being at increased risk of developing or suffering from CHD, and/or
wherein the cardiac tissue is the great arteries and the accumulation is indicative for the subject being at increased risk of developing or suffering from CHD,
preferably, wherein the cardiac tissue is the aortic valve and the accumulation is indicative for the subject being at increased risk of developing or suffering from CAVD.

24. A kit comprising the binding molecule, the nucleic acid, the nucleic acid probe, the primer, the primer pair, the biotinylated primer, the SNP microarray, the single-stranded oligonucleotide probe of item 16 or the substance of any one of items 19 to 23.

25. Use of the kit as defined in item 24 for carrying out the method or use as defined in any one of items 8 to 16 and 19 to 23.

26. The substance of any one of items 19 to 23.

27. The substance of any one of items 19 to 23 for use in in vivo diagnostics.

The disclosures in the context of the methods of the invention described herein are applicable to the corresponding uses and vice versa.

As mentioned above the invention relates to the treatment of a disease associated with cardiovascular calcification by inhibition of TLR3. Accordingly, the invention relates to an inhibitor of toll-like receptor 3 (TLR3) for use in the treatment of a disease associated with cardiovascular calcification.

Furthermore, the invention relates to a method of treating a disease associated with cardiovascular calcification, the method comprising administering an inhibitor of toll-like receptor 3.

TLR3 is a member of the Toll-like receptor (TLR) family which plays a fundamental role in pathogen recognition and activation of innate immunity. TLRs are highly conserved from Drosophila to humans and share structural and functional similarities. They recognize pathogen-associated molecular patterns (PAMPs) that are expressed on infectious agents and mediate the production of cytokines necessary for the development of effective immunity. TLR3 is an intracellular receptor described in the prior art to recognize dsRNA associated with viral infection in its function as a receptor of the innate immune system and also recognizes the synthetic dsRNA analog polyinosinic-polycytidylic acid (poly I:C). TLR3 is preferentially located within the membrane of the early endosome and comprises an ectodomain extending into the endosomal lumen, a transmembrane domain and a cytoplasmic domain.

An exemplary amino acid sequence of human TLR3 is shown hereinbelow and in SEQ ID NO: 9 and can be retrieved form the corresponding databasis, such as NCBI, EMBL, Uniprot etc.:

>sp|O15455|TLR3_HUMAN Toll-like receptor 3 OS = Homo sapiens
OX = 9606 GN = TLR3 PE = 1 SV = 1
MRQTLPCIYFWGGLLPFGMLCASSTTKCTVSHEVADCSHLKLTQVPDDLPTNITVLNLTH
NQLRRLPAANFTRYSQLTSLDVGFNTISKLEPELCQKLPMLKVLNLQHNELSQLSDKTFA
FCTNLTELHLMSNSIQKIKNNPFVKQKNLITLDLSHNGLSSTKLGTQVQLENLQELLLSN
NKIQALKSEELDIFANSSLKKLELSSNQIKEFSPGCFHAIGRLFGLFLNNVQLGPSLTEK
LCLELANTSIRNLSLSNSQLSTTSNTTFLGLKWTNLTMLDLSYNNLNVVGNDSFAWLPQL
EYFFLEYNNIQHLFSHSLHGLFNVRYLNLKRSFTKQSISLASLPKIDDFSFQWLKCLEHL
NMEDNDIPGIKSNMFTGLINLKYLSLSNSFTSLRTLTNETFVSLAHSPLHILNLTKNKIS
KIESDAFSWLGHLEVLDLGLNEIGQELTGQEWRGLENIFEIYLSYNKYLQLTRNSFALVP
SLQRLMLRRVALKNVDSSPSPFQPLRNLTILDLSNNNIANINDDMLEGLEKLEILDLQHN
NLARLWKHANPGGPIYFLKGLSHLHILNLESNGFDEIPVEVFKDLFELKIIDLGLNNLNT
LPASVFNNQVSLKSLNLQKNLITSVEKKVFGPAFRNLTELDMRFNPFDCTCESIAWFVNW
INETHTNIPELSSHYLCNTPPHYHGFPVRLFDTSSCKDSAPFELFFMINTSILLIFIFIV
LLIHFEGWRISFYWNVSVHRVLGFKEIDRQTEQFEYAAYIIHAYKDKDWVWEHFSSMEKE
DQSLKFCLEERDFEAGVFELEAIVNSIKRSRKIIFVITHHLLKDPLCKRFKVHHAVQQAI
EQNLDSIILVFLEEIPDYKLNHALCLRRGMFKSHCILNWPVQKERIGAFRHKLQVALGSK
NSVH

The above sequence SEQ ID NO: 9 was retrieved from UniProt with accession number UniProtKB—O15455 (TLR3_HUMAN). The term “TLR3” as used herein also covers variants thereof, as well as orthologs, e.g. murine TLR3. Corresponding nucleotide sequences can also be retrieved from the corresponding databases.

It is preferred herein that the patient/subject is human. Accordingly, it is preferred that the TLR3 inhibitor is an inhibitor of human TLR3, particularly when the patient/subject is human.

In one aspect, the present invention relates to the inhibition of TLR3 activity in a mechanism that induces calcification of cardiovascular tissue.

The present invention provides inhibitors of TLR3 for use in the treatment of a disease associated with cardiovascular calcification. It is envisaged herein that these inhibitors may be used as a medicament, i.e. the inhibitors of TLR3 provided and described herein are for use in medicine (e.g. for use in the therapy/treatment of a disease, in particular a disease associated with cardiovascular calcification, such as CAVD and CHD). The terms “medicament” and “pharmaceutical composition” are used interchangeably herein. Accordingly, definitions and explanations provided herein in relation to “pharmaceutical compositions”, apply, mutatis mutandis, to the term “medicament”.

The term “inhibitor” implies no specific mechanism of biological action whatsoever and is deemed to expressly include and encompass all possible pharmacological, physiological, and biochemical interactions with TLR3 whether direct or indirect. For the purpose of the present disclosure, it will be explicitly understood that the term “inhibitor” encompasses all the previously identified terms, titles, and functional states and characteristics whereby TLR3 itself, a biological activity of TLR3 (including but not limited to its ability to induce calcification processes), or the consequences of the biological activity, are substantially nullified, decreased, or neutralized in any meaningful degree, e.g., by at least 5%, 10%, 20%, 50%, 70%, 85%, 90%, 100%, 150%, 200%, 300%, 500%, or by 2-fold, 3-fold, 4-fold, 5-fold, 10-fold, 20-fold, 50-fold, 100-fold or 1000-fold. An inhibitor may decrease an abnormal level of a biological activity of TLR3 that may cause an adverse effect and/or a disease in a subject to a level that corresponds to a level in a healthy subject thereby preventing, preventing progression and/or curing the adverse effect and/or disease.

Accordingly, the present invention relates in one aspect to an inhibitor of toll-like receptor 3 (TLR3) for use in the treatment of a disease associated with cardiovascular calcification, wherein the inhibitor decreases the biological activity of TLR3 at least 2-fold, 3-fold, 4-fold, 5-fold, 10-fold, 20-fold, 50-fold, 100-fold or 1000-fold.

Accordingly, the present invention relates in one aspect to an inhibitor of toll-like receptor 3 (TLR3) for use in the treatment of a disease associated with cardiovascular calcification, wherein the inhibitor decreases an abnormal level of a biological activity of TLR3 that causes/and or promotes an adverse effect and/or a disease at least 2-fold, 3-fold, 4-fold, 5-fold, 10-fold, 20-fold, 50-fold, 100-fold or 1000-fold.

Accordingly, the present invention relates in one aspect to an inhibitor of toll-like receptor 3 (TLR3) for use in the treatment of a disease associated with cardiovascular calcification, wherein the inhibitor decreases an abnormal level of a biological activity of TLR3 that causes/and or promotes an adverse effect and/or a disease at least 3-fold.

Accordingly, the present invention relates in one aspect to an inhibitor of toll-like receptor 3 (TLR3) for use in the treatment of a disease associated with cardiovascular calcification, wherein the inhibitor decreases an abnormal level of a biological activity of TLR3 that causes/and or promotes calcific aortic valve disease (CAVD) and/or coronary heart disease (CHD).

Accordingly, the present invention relates in one aspect to an inhibitor of toll-like receptor 3 (TLR3) for use in the treatment of a disease associated with cardiovascular calcification, wherein the inhibitor decreases an abnormal level of a biological activity of TLR3 that causes/and or promotes calcific aortic valve disease (CAVD).

Accordingly, the present invention relates in one aspect to an inhibitor of toll-like receptor 3 (TLR3) for use in the treatment of a disease associated with cardiovascular calcification, wherein the inhibitor decreases an abnormal level of a biological activity of TLR3 that causes/and or promotes calcific aortic valve disease (CAVD) in a subject to a level comparable to a healthy subject.

The present invention also relates to a method of treating a disease associated with cardiovascular calcification, the method comprising administering an inhibitor of toll-like receptor 3 (TLR3), wherein the inhibitor decreases the biological activity of TLR3 at least 2-fold, 3-fold, 4-fold, 5-fold, 10-fold, 20-fold, 50-fold, 100-fold or 1000-fold.

Accordingly, the present invention relates in one aspect to a method of treating a disease associated with cardiovascular calcification, the method comprising administering an inhibitor of toll-like receptor 3 (TLR3), wherein the inhibitor decreases an abnormal level of a biological activity of TLR3 that causes and/or promotes an adverse effect and/or a disease at least 2-fold, 3-fold, 4-fold, 5-fold, 10-fold, 20-fold, 50-fold, 100-fold or 1000-fold.

Accordingly, the present invention relates in one aspect to a method of treating a disease associated with cardiovascular calcification, the method comprising administering an inhibitor of toll-like receptor 3 (TLR3), wherein the inhibitor decreases an abnormal level of a biological activity of TLR3 that causes and/or promotes an adverse effect and/or a disease at least 3-fold.

Accordingly, the present invention relates in one aspect to a method of treating a disease associated with cardiovascular calcification, the method comprising administering an inhibitor of toll-like receptor 3 (TLR3), wherein the inhibitor decreases an abnormal level of a biological activity of TLR3 that causes and/or promotes calcific aortic valve disease (CAVD) and/or coronary heart disease (CHD).

Accordingly, the present invention relates in one aspect to a method of treating a disease associated with cardiovascular calcification, the method comprising administering an inhibitor of toll-like receptor 3 (TLR3), wherein the inhibitor decreases an abnormal level of a biological activity of TLR3 that causes and/or promotes calcific aortic valve disease (CAVD).

Accordingly, the present invention relates in one aspect to a method of treating a disease associated with cardiovascular calcification, the method comprising administering an inhibitor of toll-like receptor 3 (TLR3), wherein the inhibitor decreases an abnormal level of a biological activity of TLR3 that causes and/or promotes calcific aortic valve disease (CAVD) in a subject to a level comparable to a healthy subject.

According to the present invention the term “inhibitor of toll-like receptor 3 (TLR3)” also means a compound or substance capable of fully or partially preventing or reducing the physiologic activity of TLR3. In the context of the present invention said inhibitor may therefore prevent, reduce, inhibit or inactivate the physiological activity of TLR3 e.g. upon binding of said compound/substance (i.e. of the inhibitor) to TLR3.

As used herein, the term “inhibitor” also encompasses inhibitors that result in a reversible inhibition such as competitive inhibition, uncompetitive inhibition, non-competitive inhibition, mixed inhibition or in an irreversible inhibition of TLR3, e.g. inhibition by covalent interaction. A “inhibitor of TLR3” may also be capable of preventing the function of TLR3 by preventing/reducing the expression of the nucleic acid molecule encoding for TLR3. Thus, an inhibitor of TLR3 may lead to a decreased expression level of TLR3 gene products such as a decreased level of TLR3 mRNA and/or TLR3 protein. An inhibitor of TLR3 may reduce an abnormal expression level of TLR3 that may cause and/or promote an adverse effect and/or disease associated with cardiovascular calcification in a subject to a level that corresponds to a level in a healthy subject thereby preventing, preventing progression or curing the adverse effect and/or disease in a subject. This may be reflected in a decreased TLR3 expression and/or a decreased abnormal TLR3 expression thereby restoring a healthy level of TLR3 expression. TLR3 expression level may to some extent correlate with TLR3 activity until saturation of the translation machinery and or a substance that binds TLR3 is achieved. The expression level of TLR3 may be measured/detected by methods known in the art.

Accordingly, the present invention relates in one aspect to an inhibitor of toll-like receptor 3 (TLR3) for use in the treatment of a disease associated with cardiovascular calcification, wherein the inhibitor decreases the expression level of TLR3 gene products.

Accordingly, the present invention relates in one aspect to an inhibitor of toll-like receptor 3 (TLR3) for use in the treatment of a disease associated with cardiovascular calcification, wherein the inhibitor decreases an abnormal expression level of TLR3 gene products that causes and/or promotes an adverse effect and/or a disease.

Accordingly, the present invention relates in one aspect to an inhibitor of toll-like receptor 3 (TLR3) for use in the treatment of a disease associated with cardiovascular calcification, wherein the inhibitor decreases an abnormal expression level of TLR3 gene products that causes and/or promotes CAVD and/or CHD.

Accordingly, the present invention relates in one aspect to an inhibitor of toll-like receptor 3 (TLR3) for use in the treatment of a disease associated with cardiovascular calcification, wherein the inhibitor decreases an abnormal expression level of TLR3 gene products that causes and/or promotes CAVD.

Accordingly, the present invention relates in one aspect to an inhibitor of toll-like receptor 3 (TLR3) for use in the treatment of a disease associated with cardiovascular calcification, wherein the inhibitor decreases an abnormal expression level of TLR3 gene products that causes and/or promotes CAVD in a subject to a level of expression comparable to a healthy subject.

The present invention also relates to a method of treating a disease associated with cardiovascular calcification, the method comprising administering an inhibitor of toll-like receptor 3 (TLR3), wherein the inhibitor decreases the expression level of TLR3 gene products. Accordingly, the present invention relates in one aspect to a method of treating a disease associated with cardiovascular calcification, the method comprising administering an inhibitor of toll-like receptor 3 (TLR3), wherein the inhibitor decreases an abnormal expression level of TLR3 gene products that causes and/or promotes an adverse effect and/or a disease.

Accordingly, the present invention relates in one aspect to a method of treating a disease associated with cardiovascular calcification, the method comprising administering an inhibitor of toll-like receptor 3 (TLR3), wherein the inhibitor decreases an abnormal expression level of TLR3 gene products that causes and/or promotes CAVD and/or CHD.

Accordingly, the present invention relates in one aspect to a method of treating a disease associated with cardiovascular calcification, the method comprising administering an inhibitor of toll-like receptor 3 (TLR3), wherein the inhibitor decreases an abnormal expression level of TLR3 gene products that causes and/or promotes CAVD.

Accordingly, the present invention relates in one aspect to a method of treating a disease associated with cardiovascular calcification, the method comprising administering an inhibitor of toll-like receptor 3 (TLR3), wherein the inhibitor decreases an abnormal expression level of TLR3 gene products that causes and/or promotes CAVD in a subject to a level of expression comparable to a healthy subject.

An inhibitor of TLR3 may perform its inhibitory function by directly interacting the TLR3 protein, i.e. with any part of the TLR3 protein such as the ectodomain, transmembrane domain and/or cytoplasmic domain of TLR3. An inhibitor of TLR3 may also perform any inhibitory effect on TLR3 function by preventing, reducing, inhibiting or inactivating any upstream or downstream pathway components that essentially contribute to TLR3 function. An inhibitor of TLR3 may perform any indirect inhibitory effect on any TLR activating molecules such as nucleic acids, ribonucleic acid (RNA), double-stranded ribonucleic acid (dsRNA), chromatin reader proteins and/or ligands.

Efficacy of a TLR3 inhibitor may be described using the half-maximal inhibitory concentration (IC50) value. In the sense of the present invention, a TLR3 inhibitor preferably embodies a low IC50 value. The IC50 value of a TLR3 inhibitor may be below 100 μM, below 90 μM, below 80 μM, below 70 μM, below 60 μM, below 50 μM, below 40 μM, below 30 μM, below 20 μM or below 10 μM, wherein lower values are preferred over higher values. Preferably, the IC50 value of a TLR3 inhibitor may be below 10 μm, below 9 UM, below 8 μM, below 7 μM, below 6 μM, below 5 UM or below 4 μM. Preferably, the IC50 value of a TLR3 inhibitor may be below 4 μM.

Accordingly, the invention relates to an inhibitor of toll-like receptor 3 (TLR3) for use in the treatment of a disease associated with cardiovascular calcification, wherein the efficacy of the half-maximal inhibitory concentration (IC50) is below 100 μM, below 90 μM, below 80 μM, below 70 μM, below 60 μM, below 50 μM, below 40 μM, below 30 μM, below 20 UM or below 10 μM, below 9 μM, below 8 μM, below 7 μM, below 6 μM, below 5 UM or below 4 μM, preferably below 4 μM.

Accordingly, the invention relates to (R)-2-(3-Chloro-6-fluorobenzo [b] thiophene-2-carboxamido)-3-phenyl-propanoic acid for use in the treatment of a disease associated with cardiovascular calcification, wherein the efficacy of the half-maximal inhibitory concentration (IC50) is below 4 μM.

Accordingly, the invention relates to (R)-2-(3-Chloro-6-fluorobenzo [b] thiophene-2-carboxamido)-3-phenyl-propanoic acid for use in the treatment of a disease associated with calcification of the aortic valve and/or calcification of the coronary arteries and/or calcification of the great arteries, wherein the efficacy of the half-maximal inhibitory concentration (IC50) is below 4 μM.

Accordingly, the invention relates to (R)-2-(3-Chloro-6-fluorobenzo [b] thiophene-2-carboxamido)-3-phenyl-propanoic acid for use in the treatment of a disease associated with calcification of the aortic valve, wherein the efficacy of the half-maximal inhibitory concentration (IC50) is below 4 μM.

Accordingly, the invention relates to (R)-2-(3-Chloro-6-fluorobenzo [b] thiophene-2-carboxamido)-3-phenyl-propanoic acid for use in the treatment of calcific aortic valve disease (CAVD), wherein the efficacy of the half-maximal inhibitory concentration (IC50) is below 4 μM.

Accordingly, the invention relates to (R)-2-(3-Chloro-6-fluorobenzo [b] thiophene-2-carboxamido)-3-phenyl-propanoic acid for use in the treatment of coronary heart disease (CHD), wherein the efficacy of the half-maximal inhibitory concentration (IC50) is below 4 μM.

The invention also relates to a method of treating a disease associated with cardiovascular calcification, the method comprising administering an inhibitor of toll-like receptor 3 (TLR3), wherein the efficacy of the half-maximal inhibitory concentration (IC50) is below 100 μM, below 90 μM, below 80 μM, below 70 μM, below 60 μM, below 50 μM, below 40 μM, below 30 μM, below 20 μM or below 10 μM, below 9 UM, below 8 μM, below 7 UM, below 6 μM, below 5 UM or below 4 μM, preferably below 4 μM.

Accordingly, the invention relates to a method of treating a disease associated with cardiovascular calcification, the method comprising administering (R)-2-(3-Chloro-6-fluorobenzo [b] thiophene-2-carboxamido)-3-phenyl-propanoic acid, wherein the efficacy of the half-maximal inhibitory concentration (IC50) is below 4 μM.

Accordingly, the invention relates to a method of treating a disease associated with calcification of the aortic valve and/or calcification of the coronary arteries and/or calcification of the great arteries, the method comprising administering (R)-2-(3-Chloro-6-fluorobenzo [b] thiophene-2-carboxamido)-3-phenyl-propanoic acid, wherein the efficacy of the half-maximal inhibitory concentration (IC50) is below 4 μM.

Accordingly, the invention relates to a method of treating a disease associated with calcification of the aortic valve, the method comprising administering (R)-2-(3-Chloro-6-fluorobenzo [b] thiophene-2-carboxamido)-3-phenyl-propanoic acid, wherein the efficacy of the half-maximal inhibitory concentration (IC50) is below 4 μM.

Accordingly, the invention relates to a method of treating calcific aortic valve disease (CAVD), the method comprising administering (R)-2-(3-Chloro-6-fluorobenzo [b] thiophene-2-carboxamido)-3-phenyl-propanoic acid, wherein the efficacy of the half-maximal inhibitory concentration (IC50) is below 4 μM.

Accordingly, the invention relates to a method of treating coronary heart disease (CHD), the method comprising administering (R)-2-(3-Chloro-6-fluorobenzo [b] thiophene-2-carboxamido)-3-phenyl-propanoic acid, wherein the efficacy of the half-maximal inhibitory concentration (IC50) is below 4 μM.

The skilled person is aware how to determine the IC50 value of a TLR3 inhibitor. It is envisioned herein, that TLR3 inhibitors may embody additional IC50 values and/or that other TLR3 inhibitors with other IC50 values are identified.

An inhibitor of TLR3 may be any substance that prevents, reduces, inhibits or inactivates the physiological activity of TLR3 such as a small molecule, an antibody, a nucleic acid. An inhibitor of TLR3 may be any substance that prevents, reduces, inhibits or inactivates the physiological activity of TLR3 such as a drug and/or a medicament. A drug and/or medicament may comprise any TLR3 inhibitor, e.g. a small molecule, an antibody, a nucleic acid. An inhibitor of TLR3 may be a substance such as a thiophenecarboxamidopropionate compound, an antihistamine, a selective serotonin reuptake inhibitor (SSRI), a typical antipsychotic of the phenothiazine class, a bromodomain-containing protein inhibitor, a 4-aminoquinoline and/or a class III antiarrhythmic agent.

Accordingly, the present invention relates in one aspect to an inhibitor of toll-like receptor 3 (TLR3) for use in the treatment of a disease associated with cardiovascular calcification, wherein the inhibitor is one or more of a thiophenecarboxamidopropionate compound, an antihistamine, a selective serotonin reuptake inhibitor (SSRI), a typical antipsychotic of the phenothiazine class, a bromodomain-containing protein inhibitor, a 4-aminoquinoline and/or a class III antiarrhythmic agent.

The present invention also relates to a method of treating a disease associated with cardiovascular calcification, the method comprising administering an inhibitor of toll-like receptor 3 (TLR3), wherein the inhibitor is one or more of a thiophenecarboxamidopropionate compound, an antihistamine, a selective serotonin reuptake inhibitor (SSRI), a typical antipsychotic of the phenothiazine class, a bromodomain-containing protein inhibitor, a 4-aminoquinoline and/or a class III antiarrhythmic agent.

An inhibitor of TLR3 may selectively inhibit TLR3 function/signaling without affecting related signaling pathways such as TLR1/2, TLR2/6, TLR4 and TLR7 pathways. An inhibitor of TLR3 may also selectively inhibit TLR3 function/signaling without affecting related signaling pathways such as TLR1/2, TLR2/6, TLR4 and TLR7 pathways in such a manner to cause adverse effects in a subject.

Accordingly, the invention relates to an inhibitor of toll-like receptor 3 (TLR3) for use in the treatment of a disease associated with cardiovascular calcification, wherein the inhibitor does not affect the signalling pathways of TLR1/2, TLR2/6, TLR4 and TLR7.

The present invention also relates to a method of treating a disease associated with cardiovascular calcification, the method comprising administering an inhibitor of toll-like receptor 3 (TLR3), wherein the inhibitor does not affect the signalling pathways of TLR1/2, TLR2/6, TLR4 and TLR7.

An inhibitor of TLR3 preferably exhibits minimal cytotoxicity. An inhibitor of TLR3 may also not significantly affect the viability of cells in a negative way. In general, a preferred risk/reward ratio of a TLR3 inhibitor for use in the treatment of a disease associated with cardiovascular calcification is <1. Accordingly, a preferred risk/reward ratio in a method of treating a disease associated with cardiovascular calcification, the method comprising administering an inhibitor of toll-like receptor 3 (TLR3) is <1.

Accordingly, the present invention relates in one aspect to an inhibitor of toll-like receptor 3 (TLR3) for use in the treatment of a disease associated with cardiovascular calcification, wherein the inhibitor does only cause a minimal cytotoxic effect. Accordingly, the present invention relates in one aspect to an inhibitor of toll-like receptor 3 (TLR3) for use in the treatment of a disease associated with cardiovascular calcification, wherein the inhibitor does not cause a cytotoxic effect.

The present invention also relates to a method of treating a disease associated with cardiovascular calcification, the method comprising administering an inhibitor of toll-like receptor 3 (TLR3), wherein the inhibitor does only cause a minimal cytotoxic effect.

Accordingly, the present invention relates in one aspect to a method of treating a disease associated with cardiovascular calcification, the method comprising administering an inhibitor of toll-like receptor 3 (TLR3), wherein the inhibitor does not cause a cytotoxic effect.

An inhibitor of TLR3 may be one or more of (R)-2-(3-Chloro-6-fluorobenzo [b] thiophene-2-carboxamido)-3-phenyl-propanoic acid], Levocetirizine, ORF 1329L, Sertraline, Fluphenazine, ZL0420, ZL0454, Quinacrine, Chloroquine and Amiodarone. Preferably the inhibitor of TLR3 is (R)-2-(3-Chloro-6-fluorobenzo [b] thiophene-2-carboxamido)-3-phenyl-propanoic acid.

Accordingly, the invention relates to (R)-2-(3-Chloro-6-fluorobenzo [b] thiophene-2-carboxamido)-3-phenyl-propanoic acid for use in the treatment of a disease associated with cardiovascular calcification.

The invention also relates to (R)-2-(3-Chloro-6-fluorobenzo [b] thiophene-2-carboxamido)-3-phenyl-propanoic acid for use in the treatment of a disease which is associated with calcification of the aortic valve and/or calcification of the coronary arteries and/or calcification of the great arteries.

Accordingly, invention also relates to (R)-2-(3-Chloro-6-fluorobenzo [b] thiophene-2-carboxamido)-3-phenyl-propanoic acid for use in the treatment of a disease which is associated with calcification of the aortic valve.

The invention also relates to (R)-2-(3-Chloro-6-fluorobenzo [b] thiophene-2-carboxamido)-3-phenyl-propanoic acid for use in the treatment of calcific aortic valve disease (CAVD) and/or coronary heart disease (CHD).

Accordingly, the invention relates to (R)-2-(3-Chloro-6-fluorobenzo [b] thiophene-2-carboxamido)-3-phenyl-propanoic acid for use in the treatment of calcific aortic valve disease (CAVD).

Accordingly, the invention relates to (R)-2-(3-Chloro-6-fluorobenzo [b] thiophene-2-carboxamido)-3-phenyl-propanoic acid for use in the treatment of coronary heart disease (CHD).

The invention also relates to a method of treating a disease associated with cardiovascular calcification, the method comprising administering (R)-2-(3-Chloro-6-fluorobenzo [b] thiophene-2-carboxamido)-3-phenyl-propanoic acid.

Accordingly, the invention relates to a method of treating a disease associated with calcification of the aortic valve and/or calcification of the coronary arteries and/or calcification of the great arteries, the method comprising administering (R)-2-(3-Chloro-6-fluorobenzo [b] thiophene-2-carboxamido)-3-phenyl-propanoic acid.

The invention also relates to a method of treating a disease associated with calcification of the aortic valve, the method comprising administering (R)-2-(3-Chloro-6-fluorobenzo [b] thiophene-2-carboxamido)-3-phenyl-propanoic acid.

Accordingly, the invention also relates to a method of treating calcific aortic valve disease (CAVD) and/or coronary heart disease (CHD), the method comprising administering (R)-2-(3-Chloro-6-fluorobenzo [b] thiophene-2-carboxamido)-3-phenyl-propanoic acid.

Accordingly, the invention also relates to a method of treating calcific aortic valve disease (CAVD), the method comprising administering (R)-2-(3-Chloro-6-fluorobenzo [b] thiophene-2-carboxamido)-3-phenyl-propanoic acid.

Accordingly, the invention also relates to a method of treating coronary heart disease (CHD), the method comprising administering (R)-2-(3-Chloro-6-fluorobenzo [b] thiophene-2-carboxamido)-3-phenyl-propanoic acid.

It is particularly envisaged herein that the TLR3 inhibitor selectively blocks the interaction and/or complex formation of TLR3 with TLR3 ligands, e.g. TLR3 ligands such as biglycan. Thereby the TLR3 function can be inhibited. For example, the TLR3 inhibitor (R)-2-(3-Chloro-6-fluorobenzo [b] thiophene-2-carboxamido)-3-phenyl-propanoic acid selectively blocks the interaction and/or complex formation of TLR3 with TLR3 ligands (thereby preventing TLR3 function), e.g. TLR3 ligands such as biglycan. The TLR3 inhibitor might also inhibit the interaction and/or complex formation so far unknown TLR3 ligands with TLR3. The skilled person knows how to identify additional ligands of TLR3 and how to test their interaction and/or complex formation with TLR3.

Screens to identify additional TLR3 inhibitors such as small molecule inhibitors or the like are known in the art and can be performed according to, e.g. Cheng et al. 2011.

As already mentioned above an inhibitor of TLR3 may also be an antibody. Preferably, the antibody is an antibody against human TLR3, i.e. the antibody is an inhibitory antibody against human TLR3. An antibody (interchangeably used in plural form) as used herein is an immunoglobulin molecule capable of specific binding to a target, such as a carbohydrate, polynucleotide, lipid, polypeptide, etc., through at least one antigen recognition site, located in the variable region of the immunoglobulin molecule. The preferred target herein is TLR3, particularly human TLR3. As used herein, the term “antibody” encompasses not only intact (i.e., full-length) monoclonal antibodies, but also antigen-binding fragments (such as Fab, Fab′, F(ab′)2, Fv, single chain variable fragment (scFv)), mutants thereof, fusion proteins comprising an antibody portion, humanized antibodies, chimeric antibodies, diabodies, linear antibodies, single chain antibodies, single domain antibodies (e.g., camel or llama VHH antibodies), multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a TLR3 epitope is an antibody that binds this TLR3 epitope with greater affinity, avidity, more readily, and/or with greater duration than it binds to other TLR3 epitopes or non-TLR3 epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

An inhibitor of TLR3 may be an anti-TLR3 specific antibody. An anti-TLR antibody is an antibody capable of binding to TLR3, which may inhibit TLR3 biological activity and/or downstream pathway components mediated by TLR3. In some examples, an anti-TLR3 antibody used in the methods described herein suppresses TLR3 biological activity by at least 10%, at least 20%, at least 30%, at least 40%, at least 50%, at least 60%, at least 70%, at least 80%, at least 90%, at least 100%, or by at least 2-fold, at least 5-fold, at least 10-fold, at least 20-fold, at least 50-fold, at least 100-fold, or at least 1000-fold.

TLR3 inhibition was successfully achieved in the prior art in experimental settings via blocking antibodies (see Bunting et al. 2011). For example, Bunting et al. 2011 identified/disclosed the monoclonal antibodies CNTO4685 and CNTO5429 referred to herein.

Accordingly, the present invention relates in one aspect to an anti-TLR3 antibody for use in the treatment of a disease associated with cardiovascular calcification.

The present invention also relates to a method of treating a disease associated with cardiovascular calcification, the method comprising administering an anti-TLR3 antibody. Anti-TLR3 antibodies are well known in the art and are commercially available. The skilled person is readily capable of choosing and/or generating suitable anti-TLR3 antibodies.

The present invention also relates to the anti-TLR3 antibody CNTO4685 and/or CNTO5429 for use in the treatment of a disease associated with cardiovascular calcification.

Accordingly, the present invention relates in one aspect to the anti-TLR3 antibody CNTO4685 and/or CNTO5429 for use in the treatment of a disease associated with calcification of the aortic valve and/or calcification of the coronary arteries and/or calcification of the great arteries.

Accordingly, the present invention relates in one aspect to the anti-TLR3 antibody CNTO4685 and/or CNTO5429 for use in the treatment of CAVD and/or CHD.

Accordingly, the present invention relates in one aspect to the anti-TLR3 antibody CNTO4685 and/or CNTO5429 for use in the treatment of CAVD.

Accordingly, the present invention relates in one aspect to the anti-TLR3 antibody CNTO5429 for use in the treatment of CAVD.

The present invention also relates to a method of treating a disease associated with cardiovascular calcification, the method comprising administering the anti-TLR3 antibody CNTO4685 and/or CNTO5429.

Accordingly, the present invention also relates to a method of treating a disease associated with calcification of the aortic valve and/or calcification of the coronary arteries and/or calcification of the great arteries, the method comprising administering the anti-TLR3 antibody CNTO4685 and/or CNTO5429.

Accordingly, the present invention also relates to a method of treating CAVD or CHD, the method comprising administering the anti-TLR3 antibody CNTO4685 and/or CNTO5429.

Accordingly, the present invention also relates to a method of treating CAVD, the method comprising administering the anti-TLR3 antibody CNTO4685 and/or CNTO5429.

Accordingly, the present invention also relates to a method of treating CAVD, the method comprising administering the anti-TLR3 antibody CNTO5429.

Screens to identify additional anti-TLR3 specific inhibiting antibodies are known in the art and can be performed according to, e.g. Groth et al. 1980.

The terms “treatment”, “treating” and the like are used herein to generally mean obtaining a desired pharmacological and/or physiological effect. The effect may be prophylactic in terms of completely or partially preventing a disease or symptom thereof and/or preventing the progression of a disease or symptom thereof. The term “treatment” as used herein may be understood to relate to any form of therapy.

Accordingly, the present invention relates in a preferred aspect to an inhibitor of toll-like receptor 3 (TLR3) for use in the prophylactic treatment of a disease associated with cardiovascular calcification.

The present invention also relates in a preferred aspect to a prophylactic method of treating a disease associated with cardiovascular calcification, the method comprising administering an inhibitor of toll-like receptor 3 (TLR3).

The effect may also be therapeutic in terms of partially or completely curing a disease and/or adverse effect (e.g. a symptom) attributed to the disease, in particular diseases associated with cardiovascular calcification. The term “treatment” as used herein covers any treatment of a disease associated with cardiovascular calcification in a subject and includes: (a) preventing a disease associated with cardiovascular calcification in a subject suspected being at risk of suffering of and/or developing and/or being predisposed to a disease associated with cardiovascular calcification; (b) inhibiting the disease and/or inhibiting (delaying) the progression of the disease associated with cardiovascular calcification in said subject, i.e. arresting its development; or (c) relieving the disease associated with cardiovascular calcification in said subject, i.e. causing regression of the disease. A subject suspected being at risk of suffering of and/or developing and/or being predisposed to a disease associated with cardiovascular calcification, such as CAVD and/or CHD, may already suffer from conditions that precede a disease associated with cardiovascular calcification, such as aortic sclerosis and/or aortic stenosis. Said subject may undergo a prophylactic treatment. The prophylactic treatment may comprise an TLR3 inhibitor. The prophylactic treatment, also referred to as prophylaxis, may comprise administering an inhibitor of TLR3. The prophylactic treatment may prevent the development and/or progression of a disease associated with cardiovascular calcification.

Accordingly, the present invention relates in one aspect to an inhibitor of toll-like receptor 3 (TLR3) for use in the treatment of a disease associated with cardiovascular calcification in a subject suspected being at risk of suffering of and/or developing and/or being predisposed to a disease associated with cardiovascular calcification.

Accordingly, the present invention relates in one aspect to an inhibitor of toll-like receptor 3 (TLR3) for use in the prophylaxis of a disease associated with cardiovascular calcification in a subject suspected being at risk of suffering of and/or developing and/or being predisposed to a disease associated with cardiovascular calcification.

Accordingly, the present invention relates in one aspect to an inhibitor of toll-like receptor 3 (TLR3) for use in the prophylaxis of CAVD and/or CHD in a subject suspected being at risk of suffering of and/or developing and/or being predisposed to CAVD and/or CHD.

Accordingly, the present invention relates in one aspect to an inhibitor of toll-like receptor 3 (TLR3) for use in the prophylaxis of CAVD in a subject suspected being at risk of suffering of and/or developing and/or being predisposed to CAVD.

The present invention also relates to a method of treating a disease associated with cardiovascular calcification, the method comprising administering an inhibitor of toll-like receptor 3 (TLR3) to a subject suspected being at risk of suffering of and/or developing and/or being predisposed to a disease associated with cardiovascular calcification.

Accordingly, the present invention relates in one aspect to a method of preventing the development and/or progression of a disease associated with cardiovascular calcification, the method comprising administering an inhibitor of toll-like receptor 3 (TLR3) to a subject suspected being at risk of suffering of and/or developing and/or being predisposed to a disease associated with cardiovascular calcification.

Accordingly, the present invention relates in one aspect to a method of preventing the development and/or progression of CAVD and/or CHD, the method comprising administering an inhibitor of toll-like receptor 3 (TLR3) to a subject suspected being at risk of suffering of and/or developing and/or being predisposed to CAVD and/or CHD.

Accordingly, the present invention relates in one aspect to a method of preventing the development and/or progression of CAVD, the method comprising administering an inhibitor of toll-like receptor 3 (TLR3) to a subject suspected being at risk of suffering of and/or developing and/or being predisposed to CAVD.

As used herein the term “cure” means relieving a disease, i.e. causing regression of the disease. The term “cure” can also mean causing a regression or relieving a subject of any symptoms that are underlying a disease, e.g. a disease associated with cardiovascular calcification. The term “cure” can also comprise relieving a subject from any conditions that are caused by an ignition disease such as a disease associated with cardiovascular calcification.

The disease associated with cardiovascular calcification may be prevented by a prophylactic treatment of said subject suspected being at risk of suffering of and/or developing and/or being predisposed to a disease associated with cardiovascular calcification predisposed to the disease associated with cardiovascular calcification, the prophylactic treatment comprising administering one or more TLR3 inhibitor and/or one or more drugs disclosed herein.

Accordingly, the present invention relates in one aspect to an inhibitor of toll-like receptor 3 (TLR3) for use in the prophylaxis of a disease associated with cardiovascular calcification in a subject suspected being at risk of suffering of and/or developing and/or being predisposed to a disease associated with cardiovascular calcification.

Accordingly, the present invention relates in one aspect to an inhibitor of toll-like receptor 3 (TLR3) for use in the prophylaxis of a CAVD and/or CHD in a subject suspected being at risk of suffering of and/or developing and/or being predisposed to CAVD and/or CHD.

Accordingly, the present invention relates in one aspect to an inhibitor of toll-like receptor 3 (TLR3) for use in the prophylaxis of CAVD in a subject suspected being at risk of suffering of and/or developing and/or being predisposed to CAVD.

The present invention also relates in one aspect to a method of preventing the development and/or progression a disease associated with cardiovascular calcification, the method comprising administering an inhibitor of toll-like receptor 3 (TLR3) to a subject suspected being at risk of suffering of and/or developing and/or being predisposed to a disease associated with cardiovascular calcification.

Accordingly, the present invention relates in one aspect to a method of preventing the development and/or progression of CAVD and/or CHD, the method comprising administering an inhibitor of toll-like receptor 3 (TLR3) to a subject suspected being at risk of suffering of and/or developing and/or being predisposed to CAVD and/or CHD.

Accordingly, the present invention relates in one aspect to a method of preventing the development and/or progression of CAVD, the method comprising administering an inhibitor of toll-like receptor 3 (TLR3) to a subject suspected being at risk of suffering of and/or developing and/or being predisposed to CAVD.

The progression of a disease associated with cardiovascular calcification may be prevented in a subject diagnosed with the disease associated with cardiovascular calcification by administering one or more TLR3 inhibitor and/or one or more drugs disclosed herein.

Accordingly, the present invention relates in one aspect to an inhibitor of toll-like receptor 3 (TLR3) for use in the treatment of a disease associated with cardiovascular calcification in a subject diagnosed with the disease associated with cardiovascular calcification.

Accordingly, the present invention relates in one aspect to an inhibitor of toll-like receptor 3 (TLR3) for use in the treatment of CAVD and/or CHD in a subject diagnosed with CAVD and/or.

Accordingly, the present invention relates in one aspect to an inhibitor of toll-like receptor 3 (TLR3) for use in the treatment of CAVD in a subject diagnosed with CAVD.

The present invention also relates to a method of treating a disease associated with cardiovascular calcification, the method comprising administering an inhibitor of toll-like receptor 3 (TLR3) to a subject diagnosed with the disease associated with cardiovascular calcification.

The present invention also relates to a method of treating CAVD and/or CHD, the method comprising administering an inhibitor of toll-like receptor 3 (TLR3) to a subject diagnosed with CAVD and/or CHD.

The present invention also relates to a method of treating CAVD, the method comprising administering an inhibitor of toll-like receptor 3 (TLR3) to a subject diagnosed with CAVD.

A TLR3 inhibitor as an active component of a pharmaceutical composition or drug may be administered to a subject suspected being at risk of suffering of and/or developing and/or being predisposed to a disease associated with cardiovascular calcification in any amount effective to prevent a disease associated with cardiovascular calcification. A TLR3 inhibitor as an active component of a pharmaceutical composition or drug may be administered to a subject suspected being at risk of suffering of and/or developing and/or being predisposed to CAVD and/or CHD in any amount effective to prevent a disease associated with cardiovascular calcification. A TLR3 inhibitor as an active component of a pharmaceutical composition or drug may be administered to a subject suspected being at risk of suffering of and/or developing and/or being predisposed to CAVD in any amount effective to prevent CAVD.

Accordingly, the present invention relates in one aspect to a drug for use in the treatment of a subject assessed to be at increased risk of developing a disease associated with cardiovascular calcification.

Accordingly, the present invention relates in one aspect to a drug for use in the treatment of subject suspected being at risk of suffering of and/or developing and/or being predisposed to a disease associated with cardiovascular calcification.

Accordingly, the present invention relates in one aspect to a drug comprising a TLR3 inhibitor as an active component for use in the treatment of subject suspected being at risk of suffering of and/or developing and/or being predisposed to a disease associated with cardiovascular calcification.

Accordingly, the present invention relates in one aspect to a drug comprising an effective amount of TLR3 inhibitor as an active component for use in the treatment of subject suspected being at risk of suffering of and/or developing and/or being predisposed to a disease associated with cardiovascular calcification.

Accordingly, the present invention relates in one aspect to a drug comprising an effective amount of TLR3 inhibitor as an active component for use in the treatment of subject suspected being at risk of suffering of and/or developing and/or being predisposed to CAVD and/or CHD.

Accordingly, the present invention relates in one aspect to a drug comprising an effective amount of TLR3 inhibitor as an active component for use in the treatment of subject suspected being at risk of suffering of and/or developing and/or being predisposed to CAVD.

Accordingly, the present invention relates in one aspect to a drug comprising an effective amount of (R)-2-(3-Chloro-6-fluorobenzo [b] thiophene-2-carboxamido)-3-phenyl-propanoic acid as an active component for use in the treatment of subject suspected being at risk of suffering of and/or developing and/or being predisposed to CAVD.

Accordingly, the present invention relates in one aspect to a drug comprising an effective amount of an anti-TLR3 antibody CNTO4685 and/or CNTO5429 as an active component for use in the treatment of subject suspected being at risk of suffering of and/or developing and/or being predisposed to CAVD.

A TLR3 inhibitor may be an active component of a pharmaceutical composition or drug that is administered to a subject diagnosed with a disease associated with cardiovascular calcification in any amount effective to arrest progression and/or cure the disease associated with cardiovascular calcification, such as CAVD and/or CHD, preferably CAVD. A TLR3 inhibitor as an active component of a pharmaceutical composition or drug may be administered to a subject diagnosed with CAVD and/or CHD in any amount effective to arrest progression and/or cure CAVD and/or CHD. A TLR3 inhibitor as an active component of a pharmaceutical composition or drug may be administered to a subject diagnosed with CAVD in any amount effective to arrest progression and/or cure CAVD. The skilled person is well aware how to determine the effective amount of a TLR3 inhibitor as an active component by routine measures. The effective amount of a TLR3 inhibitor as an active component may be determined in e.g. μg/kg body mass per day, hour etc.

Accordingly, the present invention relates in one aspect to a drug for use in the treatment of a subject assessed to be at increased risk of developing a disease associated with cardiovascular calcification.

Accordingly, the present invention relates in one aspect to a drug for use in the treatment of a subject diagnosed with a disease associated with cardiovascular calcification.

Accordingly, the present invention relates in one aspect to a drug for use in the treatment of a subject diagnosed with a disease associated with cardiovascular calcification, comprising a TLR3 inhibitor as an active component.

Accordingly, the present invention relates in one aspect to a drug for use in the treatment of a subject diagnosed with a disease associated with cardiovascular calcification, comprising a TLR3 inhibitor as an active component in any amount effective to arrest progression of the disease associated with cardiovascular calcification.

Accordingly, the present invention relates in one aspect to a drug for use in the treatment of a subject diagnosed with CAVD and/or CHD, comprising a TLR3 inhibitor as an active component in any amount effective to arrest progression of CAVD and/or CHD.

Accordingly, the present invention relates in one aspect to a drug for use in the treatment of a subject diagnosed with CAVD, comprising a TLR3 inhibitor as an active component in any amount effective to arrest progression of CAVD.

Accordingly, the present invention relates in one aspect to a drug for use in the treatment of a subject diagnosed with CAVD, the drug, comprising (R)-2-(3-Chloro-6-fluorobenzo [b] thiophene-2-carboxamido)-3-phenyl-propanoic acid as an active component in any amount effective to arrest progression of CAVD.

Accordingly, the present invention relates in one aspect to a drug for use in the treatment of a subject diagnosed with CAVD, the drug, comprising an anti-TLR3 antibody CNTO4685 and/or CNTO5429 as an active component in any amount effective to arrest progression of CAVD.

As used herein, the term “subject” means an individual. A subject may be an animal with a cardiovascular system, preferably a mammal. The subject may preferably be a human. The subject may be suspected to or suffer from additional conditions such as hypertension, diabetes mellitus, bicuspid aortic valve, smoking, dyslipidemia and/or chronic kidney disease, these conditions may induce, cause and/or promote cardiovascular calcification.

Accordingly the present invention relates t in one aspect o an inhibitor of toll-like receptor 3 (TLR3) for use in the treatment of a disease associated with cardiovascular calcification, wherein the cardiovascular calcification is induced and/or caused and/or promoted by one or more conditions selected from the group of hypertension, diabetes mellitus, dyslipidemia and chronic kidney disease.

The present invention also relates to a method of treating a disease associated with cardiovascular calcification, the method comprising administering an inhibitor of toll-like receptor 3 (TLR3), wherein the cardiovascular calcification is induced and/or caused and/or promoted by one or more conditions selected from the group of hypertension, diabetes mellitus, dyslipidemia and chronic kidney disease.

The subject may have been exposed to irradiation. The subject may have undergone adjuvant thoracic radiation. The subject may have undergone an adjuvant thoracic radiation treatment to treat a cancer. The adjuvant thoracic radiation treatment in a subject may induce cardiovascular calcification and promotes the development and/or progression of CAVD or CHD. The adjuvant thoracic radiation treatment in a subject suffering from cancer may induce cardiovascular calcification and promotes the development and/or progression of CAVD.

Accordingly, the present invention relates in one aspect to an inhibitor of toll-like receptor 3 (TLR3) for use in the treatment of a disease associated with cardiovascular calcification, wherein the cardiovascular calcification is induced and/or caused and/or promoted by irradiation.

Accordingly, the present invention relates in one aspect to an inhibitor of toll-like receptor 3 (TLR3) for use in the treatment of a disease associated with cardiovascular calcification, wherein the cardiovascular calcification is induced and/or caused and/or promoted by an adjuvant thoracic radiation treatment.

Accordingly, the present invention relates in one aspect to an inhibitor of toll-like receptor 3 (TLR3) for use in the treatment of a disease associated with cardiovascular calcification, wherein the cardiovascular calcification is induced and/or caused and/or promoted by an adjuvant thoracic radiation treatment to treat a cancer.

Accordingly, the present invention relates in one aspect to an inhibitor of toll-like receptor 3 (TLR3) for use in the treatment of CAVD and/or CHD, wherein the development and/or progression of CAVD and/or CHD is induced and/or caused and/or promoted by an adjuvant thoracic radiation treatment to treat a cancer.

Accordingly, the present invention relates in one aspect to an inhibitor of toll-like receptor 3 (TLR3) for use in the treatment of CAVD, wherein the development and/or progression of CAVD is induced and/or caused and/or promoted by an adjuvant thoracic radiation treatment to treat a cancer.

The present invention also relates to a method of treating a disease associated with cardiovascular calcification, the method comprising administering an inhibitor of toll-like receptor 3 (TLR3), wherein the cardiovascular calcification is induced and/or caused and/or promoted by irradiation.

Accordingly, the present invention relates in one aspect to a method of treating a disease associated with cardiovascular calcification, the method comprising administering an inhibitor of toll-like receptor 3 (TLR3), wherein the cardiovascular calcification is induced and/or caused and/or promoted by an adjuvant thoracic radiation treatment.

Accordingly, the present invention relates in one aspect to a method of treating a disease associated with cardiovascular calcification, the method comprising administering an inhibitor of toll-like receptor 3 (TLR3), wherein the cardiovascular calcification is induced and/or caused and/or promoted by an adjuvant thoracic radiation treatment to treat a cancer.

Accordingly, the present invention relates in one aspect to a method of treating CAVD and/or CHD, the method comprising administering an inhibitor of toll-like receptor 3 (TLR3), wherein the development and/or progression of CAVD and/or CHD is induced and/or caused and/or promoted by an adjuvant thoracic radiation treatment to treat a cancer.

Accordingly, the present invention relates in one aspect to a method of treating CAVD, the method comprising administering an inhibitor of toll-like receptor 3 (TLR3), wherein the development and/or progression of CAVD is induced and/or caused and/or promoted by an adjuvant thoracic radiation treatment to treat a cancer.

The cardiovascular calcification induced and/or caused and/or promoted by an adjuvant thoracic radiation treatment of a cancer in a subject may be prevented by administering a TLR3 inhibitor to a subject during, prior and/or after the adjuvant thoracic radiation.

Accordingly, the present invention relates in one aspect to an inhibitor of toll-like receptor 3 (TLR3) for use in the prophylactic treatment of a disease associated with cardiovascular calcification, wherein the cardiovascular calcification is induced and/or caused and/or promoted by an adjuvant thoracic radiation treatment.

Accordingly, the present invention relates in one aspect to an inhibitor of toll-like receptor 3 (TLR3) for use in the prophylactic treatment of CAVD, wherein development and/or progression of CAVD is induced and/or caused and/or promoted by an adjuvant thoracic radiation treatment.

The present invention also relates in one aspect to a prophylactic method of treating a disease associated with cardiovascular calcification, the method comprising administering an inhibitor of toll-like receptor 3 (TLR3), wherein the cardiovascular calcification is induced and/or caused and/or promoted by an adjuvant thoracic radiation treatment.

Accordingly, the present invention relates in one aspect to a prophylactic method of treating CAVD, the method comprising administering an inhibitor of toll-like receptor 3 (TLR3), wherein development and/or progression of CAVD is induced and/or caused and/or promoted by an adjuvant thoracic radiation treatment.

The cardiovascular calcification in a subject may be induced and/or caused and/or promoted by factors such as mechanical stress, bicuspid aortic valve, gender, advanced age, unhealthy diets, sedentary lifestyle, white race/ethnicity, body mass index, family history of diseases associated with cardiovascular calcification, total cholesterol level, high density lipoprotein cholesterol level, consumption of tobacco products and/or others.

Accordingly the present invention relates in one aspect to an inhibitor of toll-like receptor 3 (TLR3) for use in the treatment of a disease associated with cardiovascular calcification, wherein the cardiovascular calcification is induced and/or caused and/or promoted by one or more selected from the group of mechanical stress, gender, advanced age, unhealthy diets, sedentary lifestyle, white race/ethnicity, body mass index, family history of diseases associated with cardiovascular calcification, total cholesterol level, high density lipoprotein cholesterol level and consumption of tobacco products.

Accordingly the present invention relates in one aspect to an inhibitor of toll-like receptor 3 (TLR3) for use in the treatment of CAVD, wherein the development and/or progression of CAVD is induced and/or caused and/or promoted by one or more selected from the group of mechanical stress, gender, advanced age, unhealthy diets, sedentary lifestyle, white race/ethnicity, body mass index, family history of diseases associated with cardiovascular calcification, total cholesterol level, high density lipoprotein cholesterol level and consumption of tobacco products.

The present invention also relates to a method of treating a disease associated with cardiovascular calcification, the method comprising administering an inhibitor of toll-like receptor 3 (TLR3), wherein the cardiovascular calcification is induced and/or caused and/or promoted by one or more selected from the group of mechanical stress, gender, advanced age, unhealthy diets, sedentary lifestyle, white race/ethnicity, body mass index, family history of diseases associated with cardiovascular calcification, total cholesterol level, high density lipoprotein cholesterol level and consumption of tobacco products.

Accordingly the present invention relates in one aspect to a method of treating CAVD, the method comprising administering an inhibitor of toll-like receptor 3 (TLR3), wherein the development and/or progression of CAVD is induced and/or caused and/or promoted by one or more selected from the group of mechanical stress, gender, advanced age, unhealthy diets, sedentary lifestyle, race/ethnicity, body mass index, family history of diseases associated with cardiovascular calcification, total cholesterol level, high density lipoprotein cholesterol level and consumption of tobacco products.

The subject in any of the treatments or methods disclosed herein may be of advanced age. The subject in any of the treatments or methods disclosed herein may be at least 40, at least 50, at least 60, at least 70 or at least 80 years old. The subject suspected being at risk of suffering of and/or developing and/or being predisposed to a disease associated with cardiovascular calcification may be at least 20, at least 30, at least 40, at least 50, at least 60, at least 70, at least 80 or at least 90 years old. The subject may be between 20 and 80, between 30 and 80, between 40 and 80, between 50 and 80, between 60 and 80, between 70 and 80 years old. The subject may preferably be between 20 and 80 years old.

Accordingly the present invention relates in one aspect to an inhibitor of toll-like receptor 3 (TLR3) for use in the treatment of a disease associated with cardiovascular calcification in a subject suspected being at risk of suffering of and/or developing and/or being predisposed to a disease associated with cardiovascular calcification, wherein the subject is a human and between 20 and 80 years old.

Accordingly the present invention relates in one aspect to an inhibitor of toll-like receptor 3 (TLR3) for use in the treatment of CAVD and/or CHD in a subject suspected being at risk of suffering of and/or developing and/or being predisposed to CAVD and/or CHD, wherein the subject is a human and between 20 and 80 years old.

Accordingly the present invention relates in one aspect to an inhibitor of toll-like receptor 3 (TLR3) for use in the treatment of CAVD in a subject suspected being at risk of suffering of and/or developing and/or being predisposed to CAVD, wherein the subject is a human and between 20 and 80 years old.

The present invention also relates to a method of treating a disease associated with cardiovascular calcification, the method comprising administering an inhibitor of toll-like receptor 3 (TLR3) to a subject suspected being at risk of suffering of and/or developing and/or being predisposed to a disease associated with cardiovascular calcification, wherein the subject is a human and between 20 and 80 years old.

Accordingly the present invention relates in one aspect to a method of treating CAVD and/or CHD, the method comprising administering an inhibitor of toll-like receptor 3 (TLR3) to a subject suspected being at risk of suffering of and/or developing and/or being predisposed to CAVD and/or CHD, wherein the subject is a human and between 20 and 80 years old.

Accordingly the present invention relates in one aspect to a method of treating CAVD, the method comprising administering an inhibitor of toll-like receptor 3 (TLR3) to a subject suspected being at risk of suffering of and/or developing and/or being predisposed to CAVD, wherein the subject is a human and between 20 and 80 years old.

The present invention also relates to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present.

Accordingly, the present invention relates in one aspect to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present,
wherein the subject is a human and between 20 and 80 years old.

Accordingly, the present invention relates in one aspect to a method for assessing whether a subject is at increased risk of developing CAVD and/or CHD, said method comprising,
(i) determining in a sample from said subject whether one or more genomic sequences in or near a gene comprises one or more genetic variant; and
(ii) assessing that said subject is at increased risk of developing CAVD and/or CHD when the one or more genetic variant in the one or more genomic sequences in or near a gene is present,
wherein the subject is a human and between 20 and 80 years old.

Accordingly, the present invention relates in one aspect to a method for assessing whether a subject is at increased risk of developing CAVD, said method comprising,
(i) determining in a sample from said subject whether one or more genomic sequences in or near a gene comprises one or more genetic variant; and
(ii) assessing that said subject is at increased risk of developing CAVD when the one or more genetic variant in the one or more genomic sequences in or near a gene is present,
wherein the subject is a human and between 20 and 80 years old.

A subject diagnosed with a disease associated with cardiovascular calcification may be treated with a TLR3 inhibitor in any amount effective to arrest progression and/or cure the disease associated with cardiovascular calcification. A subject diagnosed with a disease associated with cardiovascular calcification may be at least 20, at least 30, at least 40, least 50 at least 60, at least 70, at least 80 or at least 90 years old. Preferably, a subject diagnosed with a disease associated with cardiovascular calcification may be at least at least 60, at least 70, at least 80 or at least 90 years old. The subject may be between 20 and 90, between 30 and 90, between 40 and 90, between 50 and 90, between 60 and 90, between 70 and 90 or between 75 and 90 years old. Preferably, the subject may be between 75 and 90 years old. Preferably, the subject may be at least 80 years old.

Accordingly, the present invention relates in one aspect to an inhibitor of toll-like receptor 3 (TLR3) for use in the treatment of a disease associated with cardiovascular calcification in a subject diagnosed with a disease associated with cardiovascular calcification, wherein the subject is a human and at least 60 years old, preferably at least 70 years old, more preferably at least 80 years old.

Accordingly, the present invention relates in one aspect to an inhibitor of toll-like receptor 3 (TLR3) for use in the treatment of CAVD and/or CHD in a subject diagnosed with CAVD and/or CHD, wherein the subject is a human and at least 60 years old, preferably at least 70 years old, more preferably at least 80 years old.

Accordingly, the present invention relates in one aspect to an inhibitor of toll-like receptor 3 (TLR3) for use in the treatment of CAVD in a subject diagnosed with CAVD, wherein the subject is a human and at least 60 years old, preferably at least 70 years old, more preferably at least 80 years old.

The present invention also relates to a method of treating a disease associated with cardiovascular calcification, the method comprising administering an inhibitor of toll-like receptor 3 (TLR3) to subject diagnosed with a disease associated with cardiovascular calcification, wherein the subject is a human and at least 60 years old, preferably at least 70 years old, more preferably at least 80 years old.

Accordingly, the present invention relates in one aspect to a method of treating CAVD and/or CHD, the method comprising administering an inhibitor of toll-like receptor 3 (TLR3) to subject diagnosed with CAVD and/or CHD, wherein the subject is a human and at least 60 years old, preferably at least 70 years old, more preferably at least 80 years old.

Accordingly, the present invention relates in one aspect to a method of treating CAVD, the method comprising administering an inhibitor of toll-like receptor 3 (TLR3) to subject diagnosed with CAVD, wherein the subject is a human and at least 60 years old, preferably at least 70 years old, more preferably at least 80 years old.

The present invention also relates to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the subject is a human and at least 60 years old, preferably at least 70 years old, more preferably at least 80 years old.

Accordingly, the present invention also relates to a substance for use in a method of in vivo diagnosis of CAVD and/or CHD in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from CAVD and/or CHD, wherein the subject is a human and at least 60 years old, preferably at least 70 years old, more preferably at least 80 years old.

Accordingly, the present invention also relates to a substance for use in a method of in vivo diagnosis of CAVD in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from CAVD, wherein the subject is a human and at least 60 years old, preferably at least 70 years old, more preferably at least 80 years old.

The present application also relates to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the subject is a human and at least 60 years old, preferably at least 70 years old, more preferably at least 80 years old.

Accordingly, the present application relates to a method for diagnosing in vivo CAVD and/or CHD in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from CAVD and/or CHD, wherein the subject is a human and at least 60 years old, preferably at least 70 years old, more preferably at least 80 years old.

Accordingly, the present application relates to a method for diagnosing in vivo CAVD in a subject, the method comprising,
(i) administering a substance to said subject, and
(ii) detecting if said substance accumulates in cardiac tissue,
wherein the level of accumulation of said substance in the leaflets of the aortic valve is indicative for said subject being at increased risk of developing or suffering from CAVD,
wherein the subject is a human and at least 60 years old, preferably at least 70 years old, more preferably at least 80 years old.

The term “subject” also includes domesticated animals, such as cats, dogs, etc., livestock (for example, cattle (cows), horses, pigs, sheep, goats, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). Subjects can also include, but are not limited to fish (for example, zebrafish, goldfish, tilapia, salmon, and trout), amphibians and reptiles. As used herein, a “subject” may be a “patient”.

According to the present invention the term “cardiovascular calcification” means ectopic buildup of mineral deposits, such as calcium minerals, that form plaques in cardiovascular tissues. In the sense of the present invention, “atherosclerosis” is the thickening or hardening of the arteries caused by a buildup of plaque in the inner lining of an artery. In advanced stages atherosclerosis in the cardiovascular tissue can be associated with cardiovascular calcification. Therefore, in some cases the terms “cardiovascular calcification” and “atherosclerosis in the cardiovascular tissue” may be used interchangeably. In the sense of the present invention affected cardiac tissue comprises all tissue of the cardiovascular system where ectopic mineral deposits or plaques can build up.

The affected cardiac tissue may be the coronary arteries and/or the great arteries and the disease associated with the calcification may be CHD.

Coronary heart disease, or CHD, is one of the most common heart diseases in industrialized countries. It is associated with one or more constricted coronary vessels, leading to the heart muscles' circulatory disorders and, therefore, to an undersupply of oxygen to the heart. As a result, the heart can no longer work properly, leading to heart pain and tightness in the chest. In the worst case, CHD can lead to a heart attack.

The affected cardiac tissue may preferably be the leaflets of the aortic valve and the disease associated with cardiovascular calcification may preferably be CAVD. Calcific aortic valve disease (CAVD) is a slow, progressive disorder that ranges from mild valve thickening without obstruction of blood flow, termed aortic sclerosis, to severe calcification with impaired leaflet motion, termed aortic stenosis.

In the sense of the preset invention, the terms “valvular calcification” and “calcification of the leaflets of the aortic valve” and “calcification of the aortic valve” can be used interchangeably. The tissue affected by calcification may be the leaflets of the aortic valve and/or vessels or vessel walls of the coronary arteries and/or great arteries. Tissue affected by calcification may be the leaflets of the aortic valve. The leaflets of the aortic valve can experience a buildup of ectopic mineral deposits during the progression of aortic sclerosis and/or aortic stenosis. Aortic sclerosis and/or aortic stenosis are the pathological conditions that can result in calcific aortic valve disease (CAVD). The tissues affected by calcification may be the coronary arteries and/or great arteries, ectopic buildup of mineral deposits in these tissues can result in coronary heart disease (CHD).

Accordingly, the present invention relates in one aspect to an inhibitor of toll-like receptor 3 (TLR3) for use in the treatment of CAVD, wherein the cardiac tissue affected by calcification is the leaflets of the aortic valve.

Accordingly, the present invention relates in one aspect to an inhibitor of toll-like receptor 3 (TLR3) for use in the treatment of CHD, wherein the cardiac tissue affected by calcification is the coronary arteries and/or the great arteries.

Accordingly, the present invention relates in one aspect to an inhibitor of toll-like receptor 3 (TLR3) for use in the treatment of aortic sclerosis and/or aortic stenosis to prevent the development and/or progression of CAVD and/or CHD.

Accordingly, the present invention relates in one aspect to an inhibitor of toll-like receptor 3 (TLR3) for use in the treatment of aortic sclerosis to prevent the development and/or progression of CAVD and/or CHD.

Accordingly, the present invention relates in one aspect to an inhibitor of toll-like receptor 3 (TLR3) for use in the treatment of aortic stenosis to prevent the development and/or progression of CAVD and/or CHD.

Accordingly, the present invention relates in one aspect to an inhibitor of toll-like receptor 3 (TLR3) for use in the treatment of aortic sclerosis to prevent the development and/or progression of CAVD.

Accordingly, the present invention relates in one aspect to an inhibitor of toll-like receptor 3 (TLR3) for use in the treatment of aortic stenosis to prevent the development and/or progression of CAVD.

Accordingly, the present invention relates in one aspect to (R)-2-(3-Chloro-6-fluorobenzo [b] thiophene-2-carboxamido)-3-phenyl-propanoic acid for use in the treatment of aortic sclerosis and/or aortic stenosis to prevent the development and/or progression of CAVD and/or CHD.

Accordingly, the present invention relates in one aspect to (R)-2-(3-Chloro-6-fluorobenzo [b] thiophene-2-carboxamido)-3-phenyl-propanoic acid for use in the treatment of aortic sclerosis to prevent the development and/or progression of CAVD.

Accordingly, the present invention relates in one aspect to (R)-2-(3-Chloro-6-fluorobenzo [b] thiophene-2-carboxamido)-3-phenyl-propanoic acid for use in the treatment of aortic stenosis to prevent the development and/or progression of CAVD.

The present invention also relates in one aspect to a method of treating CAVD, the method comprising administering an inhibitor of toll-like receptor 3 (TLR3), wherein the cardiac tissue affected by calcification is the leaflets of the aortic valve.

Accordingly, the present invention relates in one aspect to a method of treating CHD, the method comprising administering an inhibitor of toll-like receptor 3 (TLR3), wherein the cardiac tissue affected by calcification the coronary arteries and/or the great arteries.

Accordingly, the present invention relates in one aspect to a method of treating aortic sclerosis and/or aortic stenosis to prevent the development and/or progression of CAVD and/or CHD, the method comprising administering an inhibitor of toll-like receptor 3 (TLR3).

Accordingly, the present invention relates in one aspect to a method of treating aortic sclerosis to prevent the development and/or progression of CAVD and/or CHD, the method comprising administering an inhibitor of toll-like receptor 3 (TLR3).

Accordingly, the present invention relates in one aspect to a method of treating aortic stenosis to prevent the development and/or progression of CAVD and/or CHD, the method comprising administering an inhibitor of toll-like receptor 3 (TLR3).

Accordingly, the present invention relates in one aspect to a method of treating aortic sclerosis to prevent the development and/or progression of CAVD, the method comprising administering an inhibitor of toll-like receptor 3 (TLR3).

Accordingly, the present invention relates in one aspect to a method of treating aortic stenosis to prevent the development and/or progression of CAVD, the method comprising administering an inhibitor of toll-like receptor 3 (TLR3).

Accordingly, the present invention relates in one aspect to a method of treating aortic sclerosis and/or aortic stenosis to prevent the development and/or progression of CAVD and/or CHD, the method comprising administering (R)-2-(3-Chloro-6-fluorobenzo [b] thiophene-2-carboxamido)-3-phenyl-propanoic acid.

Accordingly, the present invention relates in one aspect to a method of treating aortic sclerosis to prevent the development and/or progression of CAVD, the method comprising administering (R)-2-(3-Chloro-6-fluorobenzo [b] thiophene-2-carboxamido)-3-phenyl-propanoic acid.

Accordingly, the present invention relates in one aspect to a method of treating aortic stenosis to prevent the development and/or progression of CAVD, the method comprising administering (R)-2-(3-Chloro-6-fluorobenzo [b] thiophene-2-carboxamido)-3-phenyl-propanoic acid.

It is envisioned herein that the presence of TLR3 in cardiac tissue may indicate that a subject is being at increased risk of developing or suffering from a disease associated with cardiovascular calcification. A substance binding to TLR3 may administered to a subject and may be detected by means of imaging technology. An accumulation of said substance may be indicative for a subject being at increased risk of developing or suffering from a disease associated with cardiovascular calcification.

The present invention also relates to a substance for use in a method of in vivo diagnosis of CAVD in a subject, the method comprising:
(i) administering the substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD.

Accordingly, the present invention relates in one aspect to a substance for use in a method of in vivo diagnosis of CHD in a subject, the method comprising:
(i) administering the substance to said subject, and
(ii) detecting if said substance accumulates in the coronary arteries and/or the great arteries,
wherein the level of accumulation of said substance in the coronary arteries and/or the great arteries is indicative for said subject being at increased risk of developing or suffering from CHD.

The present invention also relates to a method for diagnosing in vivo CAVD in a subject, the method comprising,
(i) administering a substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD.

Accordingly, the present invention also relates to a method for diagnosing in vivo CHD in a subject, the method comprising,
(i) administering a substance to said subject, and
(ii) detecting if said substance accumulates in the coronary arteries and/or the great arteries,
wherein the level of accumulation of said substance in the coronary arteries and/or the great arteries is indicative for said subject being at increased risk of developing or suffering from CHD.

Cardiovascular calcification may be induced and/or caused and/or promoted by atherosclerosis and/or factors promoting atherosclerosis such as mechanical stress, irradiation, chronic kidney disease, diabetes mellitus, dyslipidemia, hypertension, gender, advanced age, unhealthy diets, sedentary lifestyle, white race/ethnicity, body mass index, family history of diseases associated with cardiovascular calcification, total cholesterol level, high density lipoprotein cholesterol level, consumption of tobacco products and/or others. Cardiovascular calcification may be caused by mechanical stress in the cardiovascular system. Cardiovascular calcification may also be caused by hemodynamic stress. Cardiovascular calcification may further be caused by a combination of hemodynamic stress and factors promoting atherosclerosis. In addition, mechanical stress or other sources of stress/strain may lead to cellular injury, which in turn may cause and/or promote cardiovascular calcification. Cellular injury may be caused e.g. by mechanical stress such as hemodynamic stress.

Accordingly, the present invention relates in one aspect to an inhibitor of toll-like receptor 3 (TLR3) for use in the treatment of a disease associated with cardiovascular calcification, wherein the calcification is induced and/or caused and/or promoted by mechanical stress.

Accordingly, the present invention relates in one aspect to an inhibitor of toll-like receptor 3 (TLR3) for use in the treatment of a disease associated with cardiovascular calcification, wherein the calcification is induced and/or caused and/or promoted by hemodynamic stress.

Accordingly, the present invention relates in one aspect to an inhibitor of toll-like receptor 3 (TLR3) for use in the treatment of a disease associated with cardiovascular calcification, wherein the calcification is induced and/or caused and/or promoted by cellular injury.

Accordingly, the present invention relates in one aspect to an inhibitor of toll-like receptor 3 (TLR3) for use in the treatment of CAVD and/or CHD, wherein the calcification is caused by a combination of hemodynamic stress and factors promoting atherosclerosis.

Accordingly, the present invention relates in one aspect to an inhibitor of toll-like receptor 3 (TLR3) for use in the treatment of CAVD, wherein the calcification is caused by a combination of hemodynamic stress and factors promoting atherosclerosis.

The present application also relates in one aspect to a method of treating a disease associated with cardiovascular calcification, the method comprising administering an inhibitor of toll-like receptor 3 (TLR3), wherein the calcification is induced and/or caused and/or promoted by mechanical stress.

Accordingly, the present application relates in one aspect to a method of treating a disease associated with cardiovascular calcification, the method comprising administering an inhibitor of toll-like receptor 3 (TLR3), wherein the calcification is induced and/or caused and/or promoted by hemodynamic stress.

Accordingly, the present application relates in one aspect to a method of treating a disease associated with cardiovascular calcification, the method comprising administering an inhibitor of toll-like receptor 3 (TLR3), wherein the calcification is induced and/or caused and/or promoted by cellular injury.

Accordingly, the present application relates in one aspect to a method of treating CAVD and/or CHD, the method comprising administering an inhibitor of toll-like receptor 3 (TLR3), wherein the calcification is caused by a combination of hemodynamic stress and factors promoting atherosclerosis.

Accordingly, the present application relates to a method of treating CAVD, the method comprising administering an inhibitor of toll-like receptor 3 (TLR3), wherein the calcification is caused by a combination of hemodynamic stress and factors promoting atherosclerosis.

Hemodynamic stress may lead to the development of cardiovascular calcification by promoting an osteogenic response in cells of the cardiovascular system. In particular, calcification in the leaflets of the aortic valve can be induced by a mechanism that is characterized by a phenotypic switch of valvular interstitial cells (VICs) to bone-forming osteoblasts that leads to progressive aortic valve calcification and thus can result in CAVD. The inhibitor disclosed by the present invention prevents said mechanism and/or prevents the progression of said mechanism. In humans, TLRs show vessel-specific expression within the cardiovascular system, depending on their anatomical site (Pryshchep et al., 2008). It is thus assumed, that the same osteogenic response promoting mechanism is present in other tissues or cells of the cardiovascular system that are exposed to stresses such as mechanical stress and/or hemodynamic stress and/or cellular injury and express TLR3. The same mechanism may also be present to the same extent in vascular smooth muscle cells (VSMC) leading to development of atherosclerotic plaques in the cardiovascular system promoting the development of CHD.

Cardiovascular calcification may be induced by irradiation, e.g. in patients that have undergone radiotherapy in the treatment of e.g. cancer. It is envisioned that the stress caused by radiation of cells in a patient leads to cellular injury and thus to the release of TLR3 specific ligands such as biglycan and/or dsRNAs that promote the development and/or progression of cardiovascular calcification. Cardiovascular calcification can be induced by irradiation of the thoracic region such e.g. by an adjuvant thoracic radiation treatment. Such an adjuvant thoracic radiation treatment may be used to treat a cancer in a subject. Cardiovascular calcification induced by the adjuvant thoracic radiation treatment may promote the development and/or progression of CAVD and/or CHD, preferably CAVD.

Accordingly, the present invention relates in one aspect to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the cardiovascular calcification is induced and/or caused and/or promoted by irradiation.

Accordingly, the present invention relates in one aspect to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the cardiovascular calcification is induced and/or caused and/or promoted by an adjuvant thoracic radiation treatment.

Accordingly, the present invention relates in one aspect to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the cardiovascular calcification is induced and/or caused and/or promoted by an adjuvant thoracic radiation treatment of a cancer in a subject.

Accordingly, the present invention relates in one aspect to a substance for use in a method of in vivo diagnosis of CAVD and/or CHD in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from CAVD and/or CHD, wherein the cardiovascular calcification is induced and/or caused and/or promoted by an adjuvant thoracic radiation treatment of a cancer in a subject.

Accordingly, the present invention relates in one aspect to a substance for use in a method of in vivo diagnosis of CAVD in a subject, the method comprising:
(i) administering the substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD,
wherein the cardiovascular calcification is induced and/or caused and/or promoted by an adjuvant thoracic radiation treatment of a cancer in a subject.

The present invention also relates to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the cardiovascular calcification is induced and/or caused and/or promoted by irradiation.

Accordingly, the present invention also relates to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the cardiovascular calcification is induced and/or caused and/or promoted by an adjuvant thoracic radiation treatment.

Accordingly, the present invention also relates to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the cardiovascular calcification is induced and/or caused and/or promoted by an adjuvant thoracic radiation treatment of a cancer in a subject.

Accordingly, the present invention also relates to a method for diagnosing in vivo CAVD and/or CHD in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from CAVD and/or CHD, wherein the cardiovascular calcification is induced and/or caused and/or promoted by an adjuvant thoracic radiation treatment of a cancer in a subject.

Accordingly, the present invention also relates to a method for diagnosing in vivo CAVD in a subject, the method comprising,
(i) administering a substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD,
wherein the cardiovascular calcification is induced and/or caused and/or promoted by an adjuvant thoracic radiation treatment of a cancer in a subject.

Also envisaged herein is a method_for assessing whether a subject is at increased risk of developing a disease associated with cardiovascular calcification. Said method may comprise assessing whether one or more genetic variants are present in the subject.

Accordingly, the invention relates to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present.

According to the present invention the term “genetic variant” means that a specific nucleobase that may be associated with a pathological condition or predisposition is present in a genome of a subject. The terms “genetic variant” and “genetic variation” are used interchangeably herein. As far as genetic variants are concerned, the present invention relates in one aspect to the annotated human genome GRCh37 as a reference. A genetic variant may comprise any one of the nucleobases A, G, C or T. The genetic variant may correlate with a pathological condition or predisposition such as CAVD and/or CHD, preferably CAVD.

Accordingly, the present invention relates in one aspect to a method for assessing whether a subject is at increased risk of developing CAVD and/or CHD, said method comprising,
(i) determining in a sample from said subject whether one or more genomic sequences in or near a gene comprises one or more genetic variant; and
(ii) assessing that said subject is at increased risk of developing CAVD and/or CHD when the one or more genetic variant in the one or more genomic sequences in or near a gene is present.

Accordingly, the present invention relates in one aspect to a method for assessing whether a subject is at increased risk of developing CAVD, said method comprising,
(i) determining in a sample from said subject whether one or more genomic sequences in or near a gene comprises one or more genetic variant; and
(ii) assessing that said subject is at increased risk of developing CAVD when the one or more genetic variant in the one or more genomic sequences in or near a gene is present.

The genetic variant may be a nucleobase substitution of a nucleobase at position in the reference genome GRCh37 with any one of the nucleobases A, G, C or T. However, the genetic variant does not necessarily be different from a nucleobase of a reference genome such as GRCh37 as long as the present variant contributes to a pathological condition or predisposition. The genetic variant may be a small nucleotide polymorphism (SNP). The presence of a SNP may correlate with and/or may be indicative of a pathological condition or predisposition. The pathological condition or predisposition may be a disease associated with cardiovascular calcification such as CAVD and/or CHD, preferably CAVD.

Accordingly, the present invention relates in one aspect to a method for assessing whether a subject is at increased risk of developing CAVD and/or CHD, said method comprising,
(i) determining in a sample from said subject whether one or more genomic sequences in or near a gene comprises one or more SNPs; and
(ii) assessing that said subject is at increased risk of CAVD and/or CHD when the one or more SNPs in the one or more genomic sequences in or near a gene is present.

Accordingly, the present invention relates in one aspect to a method for assessing whether a subject is at increased risk of developing CAVD, said method comprising,
(i) determining in a sample from said subject whether one or more genomic sequences in or near a gene comprises one or more SNPs; and
(ii) assessing that said subject is at increased risk of CAVD when the one or more SNPs in the one or more genomic sequences in or near a gene is present.

The genetic variant may be located in the coding or noncoding region of the genomic DNA of a subject. The genetic variant may preferably be located in the genomic sequence in or near a gene. The genetic variant may be located in an intron. The genetic variant may be located in an exon. The genetic variant may be located within regulatory regions in or near a gene. The genomic sequence in or near a gene comprises the genomic sequence within 1 mega base pairs upstream or downstream of a gene. A single genetic variant that correlates with and/or is indicative of a pathological condition or predisposition may be referred to as an individual genetic variant.

The term “genetic variants” in the context of the present invention also relates to a multitude of small nucleotide polymorphism that in sum may contribute to a pathological condition or predisposition. The term “genetic variants” is not limited to variants in a single gene or genomic sequence in or near a gene but can be interchangeably used to describe a landscape of SNPs on different chromosomes contributing in sum to said pathological condition or predisposition such as CAVD and/or CHD, preferably CAVD.

Genetic variants in the context of the present invention may represent a risk for a subject to contract a disease associated with cardiovascular calcification, preferably CAVD. This risk may be assessed by identifying said genetic variants in a subject e.g. by known genome sequencing methods and sequence analysis methods. Identification of individual genetic variants and/or the landscape of relevant genetic variants in a subject allows a risk assessment for said subject contracting a disease associated with cardiovascular calcification such as CAVD and/or CHD, preferably CAVD. This risk assessment comprises a risk as a fold probability to contract a disease associated with cardiovascular calcification such as CAVD and/or CHD, preferably CAVD, compared to a subject where the individual variant and/or landscape of genetic variants is not present or is different. Such a risk assessment allows a practitioner to establish a treatment plan and consider prophylactic treatment to prevent and/or prevent the progression of a disease associated with cardiovascular calcification, such as CAVD and/or CHD, preferably CAVD. Such a prophylactic treatment may be a medication with a TLR3 inhibitor disclosed herein. The risk to contract a disease associated with cardiovascular calcification such as CAVD and/or CHD, preferably CAVD, in a subject where one or more genetic variants are present may be at least 1.5-fold and at most 6-fold higher compared to a subject where the one or more genetic variant is not present or is different. For example, the risk may be 1.5, 1.6, 1.7 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5,1 5.2, 5.3, 5.4, 5.5, 5.6, 5.7, 5.8, 5.9 or 6-fold increased. In particular, the risk may be increased at least 1.5, 2, 2.5, 3, 5, 5.5 or 5.86-fold.

Accordingly, the present invention relates in one aspect to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present, wherein the increased risk is at least 1.5-fold higher compared to a subject where the one or more genetic variant is not present or is different.

Accordingly, the present invention relates in one aspect to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present, wherein the increased risk is at least 2-fold higher compared to a subject where the one or more genetic variant is not present or is different.

Accordingly, the present invention relates in one aspect to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present, wherein the increased risk is at least 3-fold higher compared to a subject where the one or more genetic variant is not present or is different.

Accordingly, the present invention relates in one aspect to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present, wherein the increased risk is at least 4-fold higher compared to a subject where the one or more genetic variant is not present or is different.

Accordingly, the present invention relates in one aspect to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present, wherein the increased risk is at least 5.5-fold higher compared to a subject where the one or more genetic variant is not present or is different.

Accordingly, the present invention relates in one aspect to a method for assessing whether a subject is at increased risk of developing a disease associated with cardiovascular calcification, said method comprising,
(i) determining in a sample from said subject whether one or more genomic sequences in or near a gene comprises one or more genetic variant; and
(ii) assessing that said subject is at increased risk of developing a disease associated with cardiovascular calcification when the one or more genetic variant in the one or more genomic sequences in or near a gene is present, wherein the increased risk is 5.86-fold higher compared to a subject where the one or more genetic variant is not present or is different.

Genetic variants such as SNPs can be identified by their unique identifier numbers such as rs numbers of the Single Nucleotide Polymorphism Database (dbSNP) of Nucleotide Sequence Variation, their position within a reference genome e.g. GRCh37 and the respective identity of the risk allele.

A genetic variant may comprise one or more of the genetic variants as described herein, e.g. item 12. The risk assessment may be performed based on the identification of an individual genetic variant. The individual genetic variant may be in the genomic sequences in or near any one of a JAK1, TLR3, IFNB1, IFNA1, XYLT1 or IFNAR1 gene and/or regulatory regions thereof. The individual genetic variant may comprise any one of genetic variants as described herein, e.g. item 12. Preferably, the individual genetic variant comprises the variant rs551992948 at position 9:21457591 comprising the nucleobase C.

Accordingly, the present invention relates in one aspect to a method for assessing whether a subject is at increased risk of developing a disease associated with cardiovascular calcification, said method comprising,
(i) determining in a sample from said subject whether a genomic sequence in or near a gene comprises a genetic variant; and
(ii) assessing that said subject is at increased risk of developing a disease associated with cardiovascular calcification when the genetic variant in the genomic sequence in a gene is present, wherein the gene is one of JAK1, TLR3, IFNB1, IFNA1, XYLT1 or IFNAR1.

Accordingly, the present invention relates in one aspect to a method for assessing whether a subject is at increased risk of developing a disease associated with cardiovascular calcification, said method comprising,
(i) determining in a sample from said subject whether a genomic sequence in or near a gene comprises a genetic variant; and
(ii) assessing that said subject is at increased risk of developing a disease associated with cardiovascular calcification when the genetic variant in the genomic sequence in a gene is present, wherein the gene is any one of JAK1, TLR3, IFNB1, IFNA1, XYLT1 or IFNAR1; and
wherein the genetic variant is any one of:
(a) variant rs143732508 at position 1:65335640 comprising the nucleobase G;
(b) variant rs564691204 at position 1:65342993 comprising the nucleobase T;
(c) variant rs528952911 at position 1:65347527 comprising the nucleobase C;
(d) variant rs146653955 at position 1:65380580 comprising the nucleobase C;
(e) variant rs548870644 at position 4:186953463 comprising the nucleobase G;
(f) variant rs184106700 at position 4:187028029 comprising the nucleobase G;
(g) variant rs569915578 at position 9:21119979 comprising the nucleobase T;
(h) variant rs755535058 at position 9:21120058 comprising the nucleobase T;
(i) variant rs551992948 at position 9:21457591 comprising the nucleobase C;
(j) variant rs118001479 at position 16:17153381 comprising the nucleobase A;
(k) variant rs550834189 at position 16:17283730 comprising the nucleobase A;
(l) variant rs531295111 at position 16:17289368 comprising the nucleobase C;
(m) variant rs62033189 at position 16:17342509 comprising the nucleobase C;
(n) variant rs34588333 at position 16:17345488 comprising the nucleobase A;
(o) variant rs936346 at position 16:17376126 comprising the nucleobase C; or
(p) variant rs554831417 at position 21:34683984 comprising the nucleobase T.

Accordingly, the present invention relates in one aspect to a method for assessing whether a subject is at increased risk of developing CAVD, said method comprising,
(i) determining in a sample from said subject whether variant rs551992948 at position 9:21457591 comprises the nucleobase C; and
(ii) assessing that said subject is at increased risk of developing CAVD when variant rs551992948 at position 9:21457591 comprising the nucleobase C is present, wherein the risk is 5.86-fold higher compared to a subject where variant rs551992948 at position 9:21457591 does not comprise the nucleobase C.

Accordingly, the present invention relates in one aspect to a method for assessing whether a subject is at increased risk of developing CAVD, said method comprising,
(i) determining in a sample from said subject whether variant rs143732508 at position 1:65335640 comprises the nucleobase G; and
(ii) assessing that said subject is at increased risk of developing CAVD when variant rs143732508 at position 1:65335640 comprising the nucleobase G is present, wherein the risk is 1.47-fold higher compared to a subject where variant rs143732508 at position 1:65335640 does not comprise the nucleobase G.

Accordingly, the present invention relates in one aspect to a method for assessing whether a subject is at increased risk of developing CAVD, said method comprising,
(i) determining in a sample from said subject whether variant rs564691204 at position 1:65342993 comprises the nucleobase T; and
(ii) assessing that said subject is at increased risk of developing CAVD when variant rs564691204 at position 1:65342993 comprising the nucleobase T is present, wherein the risk is 3.20-fold higher compared to a subject where variant rs564691204 at position 1:65342993 does not comprise the nucleobase T.

Accordingly, the present invention relates in one aspect to a method for assessing whether a subject is at increased risk of developing CAVD, said method comprising,
(i) determining in a sample from said subject whether variant rs528952911 at position 1:65347527 comprises the nucleobase C; and
(ii) assessing that said subject is at increased risk of developing CAVD when variant rs528952911 at position 1:65347527 comprising the nucleobase C is present, wherein the risk is 2.31-fold higher compared to a subject where variant rs528952911 at position 1:65347527 does not comprise the nucleobase C.

Accordingly, the present invention relates in one aspect to a method for assessing whether a subject is at increased risk of developing CAVD, said method comprising,
(i) determining in a sample from said subject whether variant rs146653955 at position 1:65380580 comprises the nucleobase C; and
(ii) assessing that said subject is at increased risk of developing CAVD when variant rs146653955 at position 1:65380580 comprising the nucleobase C is present, wherein the risk is 2.13-fold higher compared to a subject where variant rs146653955 at position 1:65380580 does not comprise the nucleobase C.

Accordingly, the present invention relates in one aspect to a method for assessing whether a subject is at increased risk of developing CAVD, said method comprising,
(i) determining in a sample from said subject whether variant rs548870644 at position 4:186953463 comprises the nucleobase G; and
(ii) assessing that said subject is at increased risk of developing CAVD when variant rs548870644 at position 4:186953463 comprising the nucleobase G is present, wherein the risk is 2.94-fold higher compared to a subject where variant rs548870644 at position 4:186953463 does not comprise the nucleobase G.

Accordingly, the present invention relates in one aspect to a method for assessing whether a subject is at increased risk of developing CAVD, said method comprising,
(i) determining in a sample from said subject whether variant rs184106700 at position 4:187028029 comprises the nucleobase G; and
(ii) assessing that said subject is at increased risk of developing CAVD when variant rs184106700 at position 4:187028029 comprising the nucleobase G is present, wherein the risk is 2.12-fold higher compared to a subject where variant rs184106700 at position 4:187028029 does not comprise the nucleobase G.

Accordingly, the present invention relates in one aspect to a method for assessing whether a subject is at increased risk of developing CAVD, said method comprising,
(i) determining in a sample from said subject whether variant rs569915578 at position 9:21119979 comprises the nucleobase T; and
(ii) assessing that said subject is at increased risk of developing CAVD when variant rs569915578 at position 9:21119979 comprising the nucleobase T is present, wherein the risk is 2.58-fold higher compared to a subject where variant rs569915578 at position 9:21119979 does not comprise the nucleobase T.

Accordingly, the present invention relates in one aspect to a method for assessing whether a subject is at increased risk of developing CAVD, said method comprising,
(i) determining in a sample from said subject whether variant rs755535058 at position 9:21120058 comprises the nucleobase T; and
(ii) assessing that said subject is at increased risk of developing CAVD when variant rs755535058 at position 9:21120058 comprising the nucleobase T is present, wherein the risk is 2.59-fold higher compared to a subject where variant rs755535058 at position 9:21120058 does not comprise the nucleobase T.

Accordingly, the present invention relates in one aspect to a method for assessing whether a subject is at increased risk of developing CAVD, said method comprising,
(i) determining in a sample from said subject whether variant rs118001479 at position 16:17153381 comprises the nucleobase A; and
(ii) assessing that said subject is at increased risk of developing CAVD when variant rs118001479 at position 16:17153381 comprising the nucleobase A is present, wherein the risk is 1.51-fold higher compared to a subject where variant rs118001479 at position 16:17153381 does not comprise the nucleobase A.

Accordingly, the present invention relates in one aspect to a method for assessing whether a subject is at increased risk of developing CAVD, said method comprising,
(i) determining in a sample from said subject whether variant rs550834189 at position 16:17283730 comprises the nucleobase A; and
(ii) assessing that said subject is at increased risk of developing CAVD when variant rs550834189 at position 16:17283730 comprising the nucleobase A is present, wherein the risk is 1.57-fold higher compared to a subject where variant rs550834189 at position 16:17283730 does not comprise the nucleobase A.

Accordingly, the present invention relates in one aspect to a method for assessing whether a subject is at increased risk of developing CAVD, said method comprising,
(i) determining in a sample from said subject whether variant rs531295111 at position 16:17289368 comprises the nucleobase C; and
(ii) assessing that said subject is at increased risk of developing CAVD when variant rs531295111 at position 16:17289368 comprising the nucleobase C is present, wherein the risk is 2.11-fold higher compared to a subject where variant rs531295111 at position 16:17289368 does not comprise the nucleobase C.

Accordingly, the present invention relates in one aspect to a method for assessing whether a subject is at increased risk of developing CAVD, said method comprising,
(i) determining in a sample from said subject whether variant rs62033189 at position 16:17342509 comprises the nucleobase C; and
(ii) assessing that said subject is at increased risk of developing CAVD when variant rs62033189 at position 16:17342509 comprising the nucleobase C is present, wherein the risk is 1.09-fold higher compared to a subject where variant rs62033189 at position 16:17342509 does not comprise the nucleobase C.

Accordingly, the present invention relates in one aspect to a method for assessing whether a subject is at increased risk of developing CAVD, said method comprising,
(i) determining in a sample from said subject whether variant rs34588333 at position 16:17345488 comprises the nucleobase A; and
(ii) assessing that said subject is at increased risk of developing CAVD when variant rs34588333 at position 16:17345488 comprising the nucleobase A is present, wherein the risk is 1.08-fold higher compared to a subject where variant rs34588333 at position 16:17345488 does not comprise the nucleobase A.

Accordingly, the present invention relates in one aspect to a method for assessing whether a subject is at increased risk of developing CAVD, said method comprising,
(i) determining in a sample from said subject whether variant rs936346 at position 16:17376126 comprises the nucleobase C; and
(ii) assessing that said subject is at increased risk of developing CAVD when variant rs936346 at position 16:17376126 comprising the nucleobase C is present, wherein the risk is 1.07-fold higher compared to a subject where variant rs936346 at position 16:17376126 does not comprise the nucleobase C.

Accordingly, the present invention relates in one aspect to a method for assessing whether a subject is at increased risk of developing CAVD, said method comprising,
(i) determining in a sample from said subject whether variant rs554831417 at position 21:34683984 comprises the nucleobase T; and
(ii) assessing that said subject is at increased risk of developing CAVD when variant rs554831417 at position 21:34683984 comprising the nucleobase T is present, wherein the risk is 3.53-fold higher compared to a subject where variant rs554831417 at position 21:34683984 does not comprise the nucleobase T.

Additional genetic variants may be used according to the methods disclosed herein.

A landscape of genetic variants may comprise two or more genetic variants. The identity of genetic variants within a landscape of genetic variants may be specific for a risk assessment of a subject for contracting a disease associated with cardiovascular calcification such as CAVD and/or CHD, preferably CAVD. A landscape of genetic variants may comprise at least two and at most 16 genetic variants distributed over the genome of a subject. Within a landscape of genetic variants not every individual variant present necessarily contributes to, correlates with and/or is indicative of a pathological condition and/or predisposition, however the overall effect of genetic variants contributing to a fold increased risk of a pathological composition or predisposition obtained by the analysis of genetic variants present in the landscape allows the skilled person or practitioner to create a risk assessment for a subject to suffer from the pathological condition or predisposition such as CAVD or CHD, preferably CAVD. The landscape of genetic variants may comprise genetic variants in the genomic sequences in or near one or more of the JAK1, TLR3, IFNB1, IFNA1, XYLT1 or IFNAR1 genes and/or regulatory regions thereof. The landscape of genetic variants may comprise at least two of variant rs143732508 at position 1:65335640 comprising the nucleobase G, variant rs564691204 at position 1:65342993 comprising the nucleobase T, variant rs528952911 at position 1:65347527 comprising the nucleobase C, variant rs146653955 at position 1:65380580 comprising the nucleobase C, variant rs548870644 at position 4:186953463 comprising the nucleobase G, variant rs184106700 at position 4:187028029 comprising the nucleobase G, variant rs569915578 at position 9:21119979 comprising the nucleobase T, variant rs755535058 at position 9:21120058 comprising the nucleobase T, variant rs551992948 at position 9:21457591 comprising the nucleobase C, variant rs118001479 at position 16:17153381 comprising the nucleobase A, variant rs550834189 at position 16:17283730 comprising the nucleobase A, variant rs531295111 at position 16:17289368 comprising the nucleobase C, variant rs62033189 at position 16:17342509 comprising the nucleobase C, variant rs34588333 at position 16:17345488 comprising the nucleobase A, variant rs936346 at position 16:17376126 comprising the nucleobase C, variant rs554831417 at position 21:34683984 comprising the nucleobase T.

Accordingly, the present invention relates in one aspect to a method for assessing whether a subject is at increased risk of developing a disease associated with cardiovascular calcification, said method comprising,
(i) determining in a sample from said subject whether at least two genomic sequences in or near one or more genes comprises at least two genetic variants; and
(ii) assessing that said subject is at increased risk of developing a disease associated with cardiovascular calcification when the at least two genetic variants in the at least two genomic sequences in or near one or more genes is present.

Accordingly, the present invention relates in one aspect to a method for assessing whether a subject is at increased risk of developing a disease associated with cardiovascular calcification, said method comprising,
(i) determining in a sample from said subject whether at least two genomic sequences in or near one or more genes comprises at least two genetic variants; and
(ii) assessing that said subject is at increased risk of developing a disease associated with cardiovascular calcification when the at least two genetic variants in the at least two genomic sequences in or near one or more genes is present, wherein the increased overall risk is at least 1.5-fold higher compared to a subject where the least two genetic variants are not present or are different.

Genetic variants can be identified by methods known in the art, utilizing compounds such as binding molecules, nucleic acids and SNP microarrays. Nucleic acids for identification of genetic variations in form of SNPs comprise nucleic acid probes, primers, primer pairs, biotinylated primers, single-stranded oligonucleotide probes.

Accordingly, the present invention relates in one aspect to a use of a binding molecule, a nucleic acid, a nucleic acid probe, a primer, a primer pair, a biotinylated primer, a SNP microarray, a single-stranded oligonucleotide probe specific for one or more genetic variant of the one or more genomic sequences in or near a gene according to any one of the methods described herein for assessing whether a subject is at increased risk of developing a disease associated with cardiovascular calcification.

Accordingly, the present invention relates in one aspect to a use of a binding molecule, a nucleic acid, a nucleic acid probe, a primer, a primer pair, a biotinylated primer, a SNP microarray, a single-stranded oligonucleotide probe specific for one or more genetic variant of the one or more genomic sequences in or near a gene, according to the methods described herein, for assessing whether a subject is at increased risk of developing CAVD and/or CHD.

Accordingly, the present invention relates in one aspect to a use of a binding molecule, a nucleic acid, a nucleic acid probe, a primer, a primer pair, a biotinylated primer, a SNP microarray, a single-stranded oligonucleotide probe specific for one or more genetic variant of the one or more genomic sequences in or near a gene, according to the methods described herein, for assessing whether a subject is at increased risk of developing CAVD.

It is envisioned herein, that a subject assessed to be at increased risk of developing a disease associated with cardiovascular calcification according to the methods disclosed herein may be treated with a drug to prevent and/or prevent the progression of a disease associated with cardiovascular calcification. The present invention also relates to a drug for use in the treatment of a subject assessed to be at increased risk of developing a disease associated with cardiovascular calcification according to any one of the methods or uses described herein.

In the context of the present invention a drug may comprise an inhibitor of TLR3 or the active compound that inhibits TLR3 and therefore prevents and/or inhibits and/or reduces cardiovascular calcification, ultimately preventing or reducing the burden of CAVD and/or CHD. In addition, a drug may comprise pharmaceutically acceptable carriers.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions may include a pharmaceutically acceptable salt, e.g., an acid addition salt or a base addition salt.

Accordingly, the present invention relates in one aspect to a drug for use in the treatment of a subject assessed to be at increased risk of developing a disease associated with cardiovascular calcification including a pharmaceutically acceptable carrier.

The drug may be any drug used in the art to treat patients which are at (increased) risk of developing a disease associated with cardiovascular calcification. Such drugs are described herein below. They can be used alone or in co-therapy (i.e. one of these drugs combined with one or more of the other drugs and/or e.g. in combination with a TLR3 inhibitor) to treat e.g. hypertension, irradiation, chronic kidney disease, diabetes mellitus, dyslipidemia and/or consumption of tobacco products, and the like.

Such drugs (alone or in co-therapy as described above) may also be used in the treatment of subjects which are diagnosed to suffer from or diagnosed to be prone to suffering from (i.e. to be at risk of suffering from) a disease associated with cardiovascular calcification by the herein described method of in vivo diagnosis of a disease associated with cardiovascular calcification.

In one aspect, the present invention relates to a drug for use in the treatment of a subject assessed to be at increased risk of developing a disease associated with cardiovascular calcification, wherein the drug comprises an inhibitor of toll-like receptor 3 (TLR3) as an active compound.

A drug may comprise additional compounds such as medicaments for the simultaneous treatment of factors promoting cardiovascular calcification and/or factors promoting atherosclerosis such as hemodynamic stress, hypertension, irradiation, chronic kidney disease, diabetes mellitus, dyslipidemia and/or consumption of tobacco products.

Accordingly the present invention relates in one aspect to a drug for use in the treatment of a subject assessed to be at increased risk of developing a disease associated with cardiovascular calcification, wherein the drug comprises a TLR3 inhibitor and a medicament to simultaneously treat factors promoting cardiovascular calcification and/or factors promoting atherosclerosis. Accordingly the present invention relates in one aspect to a drug for use in the treatment of a subject assessed to be at increased risk of developing a disease associated with cardiovascular calcification, wherein the drug comprises a TLR3 inhibitor and a medicament to simultaneously treat hypertension, irradiation, chronic kidney disease, diabetes mellitus, dyslipidemia and/or consumption of tobacco products.

The drug may comprise as an active compound one or more of a thiophenecarboxamidopropionate compound, an antihistamine, a selective serotonin reuptake inhibitor (SSRI), a typical antipsychotic of the phenothiazine class, a bromodomain-containing protein inhibitor, a 4-aminoquinoline or a class III antiarrhythmic agent. The drug may preferably (R)-2-(3-Chloro-6-fluorobenzo [b] thiophene-2-carboxamido)-3-phenyl-propanoic acid, Levocetirizine, ORF 1329L, Sertraline, Fluphenazine, ZL0420, ZL0454, Quinacrine, Chloroquine and Amiodarone as an active compound.

Accordingly, the present invention relates in one aspect to a drug for use in the treatment of a subject assessed to be at increased risk of developing a disease associated with cardiovascular calcification, wherein the drug comprises one or more of a thiophenecarboxamidopropionate compound, an antihistamine, a selective serotonin reuptake inhibitor (SSRI), a typical antipsychotic of the phenothiazine class, a bromodomain-containing protein inhibitor, a 4-aminoquinoline or a class III antiarrhythmic agent as an active compound.

Accordingly, the present invention relates in one aspect to a drug for use in the treatment of a subject assessed to be at increased risk of developing a disease associated with cardiovascular calcification, wherein the drug comprises one or more of (R)-2-(3-Chloro-6-fluorobenzo [b] thiophene-2-carboxamido)-3-phenyl-propanoic acid, Levocetirizine, ORF 1329L, Sertraline, Fluphenazine, ZL0420, ZL0454, Quinacrine, Chloroquine and Amiodarone as an active compound.

Accordingly, the present invention relates in one aspect to a drug for use in the treatment of a subject assessed to be at increased risk of developing a disease associated with cardiovascular calcification, wherein the drug comprises (R)-2-(3-Chloro-6-fluorobenzo [b] thiophene-2-carboxamido)-3-phenyl-propanoic acid as an active compound.

Accordingly, the present invention relates in one aspect to a drug for use in the treatment of a subject assessed to be at increased risk of developing CAVD and/or CHD, wherein the drug comprises (R)-2-(3-Chloro-6-fluorobenzo [b] thiophene-2-carboxamido)-3-phenyl-propanoic acid as an active compound.

Accordingly, the present invention relates in one aspect to a drug for use in the treatment of a subject assessed to be at increased risk of developing CAVD and/or CHD or suffering from CAVD and/or CHD, wherein the drug comprises (R)-2-(3-Chloro-6-fluorobenzo [b] thiophene-2-carboxamido)-3-phenyl-propanoic acid as an active compound.

Accordingly, the present invention relates in one aspect to a drug for use in the treatment of a subject assessed to be at increased risk of developing CAVD, wherein the drug comprises (R)-2-(3-Chloro-6-fluorobenzo [b] thiophene-2-carboxamido)-3-phenyl-propanoic acid as an active compound.

Accordingly, the present invention relates in one aspect to a drug for use in the treatment of a subject assessed to be suffering from CAVD, wherein the drug comprises (R)-2-(3-Chloro-6-fluorobenzo [b] thiophene-2-carboxamido)-3-phenyl-propanoic acid as an active compound.

The present invention also relates in one aspect to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The present invention also relates to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

According to the present invention “in vivo diagnosis” relates to an early diagnosis of a disease associated with cardiovascular calcification. Cardiovascular calcification may be detected by standard imaging techniques, such as conventional transthoracic echocardiograph. However, such imaging will often diagnose the disease at a stage where successfully treatment might not be possible anymore and surgical intervention is indispensable. Thus in vivo diagnosis in the context of the present invention may be applied at a very early stage and possibly before onset of a significant pathological condition, such as aortic sclerosis and/or aortic stenosis, to prevent progression of a disease associated with cardiovascular calcification, such as CAVD and/or CHD, preferably CAVD. Further, the in vivo diagnosis may be applied at a stage where there is not yet any hemodynamic compromise of the aortic valve.

Accordingly, the present invention relates in one aspect to a substance for use in a method of in vivo diagnosis of a disease associated with cardiovascular calcification in a subject before onset of a significant pathological condition, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The present invention also relates to a substance for use in a method of in vivo diagnosis of a disease associated with cardiovascular calcification in a subject without symptoms of CAVD and/or CHD, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The present invention also relates to a substance for use in a method of in vivo diagnosis of a disease associated with cardiovascular calcification in a subject without symptoms of CAVD, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The present invention also relates to a method for diagnosing in vivo a disease associated with cardiovascular calcification in a subject before onset of a significant pathological condition, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

Accordingly, the present invention relates in one aspect to a method for diagnosing in vivo a disease associated with cardiovascular calcification in a subject without symptoms of CAVD and/or CHD, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

Accordingly, the present invention relates in one aspect to a method for diagnosing in vivo a disease associated with cardiovascular calcification in a subject without symptoms of CAVD, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The in vivo diagnosis of the present invention can be especially advantageous to diagnose a disease associated with cardiovascular calcification in subjects that have been previously identified to be at a higher risk of contracting a disease associated with cardiovascular calcification, such as CAVD and/or CHD, preferably CAVD, due to the presence of one or more genetic variants, i.e. being predisposed.

The present invention also relates to a substance for use in a method of in vivo diagnosis of a disease associated with cardiovascular calcification in a subject being predisposed to a disease associated with cardiovascular calcification, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The present invention also relates to a substance for use in a method of in vivo diagnosis of a disease associated with cardiovascular calcification in a subject being predisposed to CAVD and/or CHD, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from CAVD and/or CHD.

The present invention also relates to a substance for use in a method of in vivo diagnosis of a disease associated with cardiovascular calcification in a subject being predisposed to CAVD, the method comprising:
(i) administering the substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD.

The present invention also relates to a method for diagnosing in vivo a disease associated with cardiovascular calcification in a subject being predisposed to a disease associated with cardiovascular calcification, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification.

The present invention also relates to a method for diagnosing in vivo a disease associated with cardiovascular calcification in a subject being predisposed to CAVD and/or CHD, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from CAVD and/or CHD.

The present invention also relates to a method for diagnosing in vivo a disease associated with cardiovascular calcification in a subject being predisposed to CAVD, the method comprising,
(i) administering a substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD.

The in vivo diagnosis makes use of a substance that is able to accumulate in cardiac tissue of a subject by binding to TLR3, said accumulation may be detected by means of imaging technology. The extend of said accumulation of said substance may be indicative for a subject to develop or suffer from a disease associated with cardiovascular calcification, such as CAVD and/or CHD, preferably CAVD. Said substance may bind to TLR3. Said substance may also bind a TLR3 epitope. Said substance may be radioactively labelled to enable detection if said substance. Said substance may comprise a radioactively labelled TLR3 specific ligand, antibody or nucleic acid. The radioactively labelled TLR3 specific ligand, antibody or nucleic acid may bind TLR3. The radioactively labelled TLR3 specific ligand, antibody or nucleic acid may bind TLR3 in vivo. The TLR3 specific ligand may be radioactively labelled biglycan. The anti-TLR3 antibody may be radioactively labelled CNTO4685 and/or CNTO5429. Preferably, the anti-TLR3 antibody may be radioactively labelled CNTO5429.

The skilled person is capable of choosing suitable substances and corresponding detection methods.

Accordingly, the present invention relates in one aspect to a radioactively labelled substance that binds TLR3 for use in a method of in vivo diagnosis of a disease associated with cardiovascular calcification in a subject, the method comprising:
(i) administering the radioactively labelled substance that binds TLR3 to said subject, and
(ii) detecting if said radioactively labelled substance that binds TLR3 accumulates in cardiac tissue,
wherein the level of accumulation of said radioactively labelled substance that binds TLR3 in the cardiac tissue is indicative for said subject being at increased risk of developing or suffering from a disease associated with cardiovascular calcification.

Accordingly, the present invention relates in one aspect to a radioactively labelled TLR3 specific ligand, antibody or nucleic acid probe for use in a method of in vivo diagnosis of a disease associated with cardiovascular calcification in a subject, the method comprising:
(i) administering the radioactively labelled TLR3 specific ligand, antibody or nucleic acid probe to said subject, and
(ii) detecting if said radioactively labelled TLR3 specific ligand, antibody or nucleic acid probe accumulates in cardiac tissue,
wherein the level of accumulation of said radioactively labelled TLR3 specific ligand, antibody or nucleic acid probe in the cardiac tissue is indicative for said subject being at increased risk of developing or suffering from a disease associated with cardiovascular calcification.

The present invention also relates to a method for diagnosing in vivo a disease associated with cardiovascular calcification in a subject, the method comprising,
(i) administering a radioactively labelled substance that binds TLR3 to said subject, and
(ii) detecting if said radioactively labelled substance that binds TLR3 accumulates in cardiac tissue,
wherein the level of accumulation of said radioactively labelled substance that binds TLR3 in the cardiac tissue is indicative for said subject being at increased risk of developing or suffering from a disease associated with cardiovascular calcification.

The present invention also relates to a method for diagnosing in vivo a disease associated with cardiovascular calcification in a subject, the method comprising,
(i) administering a radioactively labelled TLR3 specific ligand, antibody or nucleic acid probe to said subject, and
(ii) detecting if said radioactively labelled TLR3 specific ligand, antibody or nucleic acid probe accumulates in cardiac tissue,
wherein the level of accumulation of said radioactively labelled TLR3 specific ligand, antibody or nucleic acid probe in the cardiac tissue is indicative for said subject being at increased risk of developing or suffering from a disease associated with cardiovascular calcification.

The accumulation of said substance may correlate with the presence of TLR3. Said accumulation may correlate with the presence of TLR3 in cardiovascular tissues such as the leaflets of the aortic valve and/or the vessels or vessel walls of the coronary arteries and/or great arteries. Said accumulation of said substance may be measured by methods known in the art. The accumulation of the radioactively labelled substance may be measured by positron emission tomography (PET). The accumulation may be measured in a subject suspected being at risk of suffering of and/or developing and/or being predisposed to a disease associated with cardiovascular calcification.

Accordingly, the present invention relates in one aspect to a substance for use in a method of in vivo diagnosis of a disease associated with cardiovascular calcification in a subject being at risk of suffering of and/or developing and/or being predisposed to CAVD and/or CHD, the method comprising:
(i) administering the substance to said subject, and
(ii) detecting if said substance accumulates in the leaflets of the aortic valve and/or the vessels or vessel walls of the coronary arteries and/or great arteries,
wherein the level of accumulation of said substance in the leaflets of the aortic valve and/or the vessels or vessel walls of the coronary arteries and/or great arteries is indicative for said subject being at increased risk of developing or suffering from CAVD and/or CHD.

Accordingly, the present invention relates in one aspect to a substance for use in a method of in vivo diagnosis of a disease associated with cardiovascular calcification in a subject being at risk of suffering of and/or developing and/or being predisposed to CAVD, the method comprising:
(i) administering the substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD.

The present invention also relates to a method for diagnosing in vivo a disease associated with cardiovascular calcification in a subject, the method comprising,
(i) administering a substance to said subject being at risk of suffering of and/or developing and/or being predisposed to CAVD and/or CHD, and
(ii) detecting if said substance accumulates in the leaflets of the aortic valve and/or the vessels or vessel walls of the coronary arteries and/or great arteries,
wherein the level of accumulation of said substance in the leaflets of the aortic valve and/or the vessels or vessel walls of the coronary arteries and/or great arteries is indicative for said subject being at increased risk of developing or suffering from CAVD and/or CHD.

Accordingly, the present invention relates in one aspect to a method for diagnosing in vivo a disease associated with cardiovascular calcification in a subject, the method comprising,
(i) administering a substance to said subject being at risk of suffering of and/or developing and/or being predisposed to CAVD, and
(ii) detecting if said substance accumulates in the leaflets of the aortic valve,
wherein the level of accumulation of said substance in the leaflets of the aortic valve is indicative for said subject being at increased risk of developing or suffering from CAVD.

The measurement of the accumulation of the substance in the cardiac tissue may be performed on said subject once and a final risk assessment of said subject is subsequently prepared by a practitioner based on the single measurement. The measurement may also be performed at least 2-times, 3-times or 4-times. The measurement results of said multiple measurements may be combined for a risk assessment of said subject by a practitioner. Time between the subsequent measurements may be at least 1 month and at most 12 months. The measurement may be performed regularly, e.g. every 12 months, 18 months, 24 months, 30 months or 36 months to monitor development of a disease associated with cardiovascular calcification in a subject suspected being at risk of suffering of and/or developing and/or being predisposed to a disease associated with cardiovascular calcification.

Accordingly, the present invention relates in one aspect to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the in vivo diagnosis is performed at least two times.

Accordingly, the present invention relates in one aspect to a substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the in vivo diagnosis is performed regularly to monitor an increased risk of developing or suffering from a disease associated with cardiovascular calcification in a subject.

Accordingly, the present invention relates in one aspect to a substance for use in a method of in vivo diagnosis of CAVD and/or CHD in a subject, the method comprising:
(i) administering the substance to said subject, and
(ii) detecting if said substance accumulates in the leaflets of the aortic valve and/or the vessels or vessel walls of the coronary arteries and/or great arteries,
wherein the level of accumulation of said substance in the leaflets of the aortic valve and/or the vessels or vessel walls of the coronary arteries and/or great arteries is indicative for said subject being at increased risk of developing or suffering from CAVD and/or CHD, wherein the in vivo diagnosis is performed regularly to monitor an increased risk of developing or suffering from CAVD and/or CHD.

Accordingly, the present invention relates in one aspect to a substance for use in a method of in vivo diagnosis of CAVD in a subject, the method comprising:
(i) administering the substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD,
wherein the in vivo diagnosis is performed regularly to monitor an increased risk of developing or suffering from CAVD.

The present invention also relates to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the in vivo diagnosis is performed at least two times.

Accordingly, the present invention relates in one aspect to a method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification, wherein the in vivo diagnosis is performed regularly to monitor an increased risk of developing or suffering from a disease associated with cardiovascular calcification in a subject.

Accordingly, the present invention relates in one aspect to a method for diagnosing in vivo CAVD and/or CHD in a subject, the method comprising,
(i) administering a substance to said subject, and
(ii) detecting if said substance accumulates in the leaflets of the aortic valve and/or the vessels or vessel walls of the coronary arteries and/or great arteries,
wherein the level of accumulation of said substance in the leaflets of the aortic valve and/or the vessels or vessel walls of the coronary arteries and/or great arteries is indicative for said subject being at increased risk of developing or suffering from CAVD and/or CHD, wherein the in vivo diagnosis is performed regularly to monitor an increased risk of developing or suffering from CAVD and/or CHD.

Accordingly, the present invention relates in one aspect to a method for diagnosing in vivo CAVD in a subject, the method comprising,
(i) administering a substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD,
wherein the in vivo diagnosis is performed regularly to monitor an increased risk of developing or suffering from CAVD.

The accumulation of the substance in a subject, which correlates with the presence of TLR3, may be compared to the amount of accumulation in a control, e.g. in one or more healthy subjects, and/or compared to a reference value (cut-off value). The amount of accumulation of said substance in one or more healthy subjects may be curated data collected from a cohort of healthy subjects, i.e. subjects suspected not being at risk of suffering of and/or developing and/or being predisposed to a disease associated with cardiovascular calcification, such as CAVD and/or CHD, preferably CAVD. Thus, the control or reference value (cut-off value), may be obtained by determining the level of accumulation in one or more healthy subjects, e.g. by using (curated) data collected from a cohort of healthy subjects. The curated data may comprise measurements of the amount of accumulation of said substance of at least 20, 30, 50, 60, 70, 80, 90, 100 or more healthy subjects. A significant increase in the amount of accumulation of said substance in a subject suspected being at risk of suffering of and/or developing and/or being predisposed to a disease associated with cardiovascular calcification may be compared to the control, e.g. the curated dataset of healthy subjects. The result of such a comparison may be indicative for the subject to be at risk of suffering of and/or developing and/or being predisposed to a disease associated with cardiovascular calcification, such as CAVD and/or CHD, preferably CAVD. An increase in the amount of accumulation of said substance in a subject suspected being at risk of suffering of and/or developing and/or being predisposed to a disease associated with cardiovascular calcification, such as CAVD and/or CHD, preferably CAVD, may be indicative for the subject to be at risk of suffering of and/or developing and/or being predisposed to a disease associated with cardiovascular calcification, such as CAVD and/or CHD, preferably CAVD.

The substance may be administered to a subject suspected being at risk of suffering of and/or developing and/or being predisposed to a disease associated with cardiovascular calcification. The substance may be administered intravenously. It is envisioned that the intravenous administration of exposes the substance that binds to TLR3 in vivo to cardiac tissues such as the leaflets of the aortic valve and/or the vessels or vessel walls of the coronary arteries and/or great arteries thereby allowing detection and measurement of the accumulation of said substance in said tissues.

Accumulation of the substance may be detected in any tissue of the cardiovascular system. The accumulation of the substance may preferably be detected the leaflets of the aortic valve, coronary arteries and/or great arteries. Accumulation of the substance may be indicative for a subject to develop and/or suffer from aortic sclerosis and/or aortic stenosis and/or coronary heart disease (CHD) and/or calcific aortic valve disease (CAVD), preferably CAVD. The accumulation may preferably be detected in the leaflets of the aortic valve where an accumulation is indicative for a subject to develop and/or suffer from aortic sclerosis and/or aortic stenosis. The accumulation may be preferably detected in the leaflets of the aortic valve where an accumulation is indicative for a subject to develop and/or suffer from CAVD. The accumulation may also be detected in the coronary arteries and/or great arteries is indicative for a subject to develop and/or suffer from CHD.

Accordingly, the present invention relates in one aspect to a substance for use in a method of in vivo diagnosis of CAVD in a subject, the method comprising:
(i) administering the substance to said subject, and
(ii) detecting if said substance accumulates the leaflets of the aortic valve,
wherein the level of accumulation of said substance in the leaflets of the aortic valve is indicative for said subject being at increased risk of developing or suffering from CAVD.

Accordingly, the present invention relates in one aspect to a substance for use in a method of in vivo diagnosis of CHD in a subject, the method comprising:
(i) administering the substance to said subject, and
(ii) detecting if said substance accumulates in the vessels or vessel walls of the coronary arteries and/or great arteries,
wherein the level of accumulation of said substance in the vessels or vessel walls of the coronary arteries and/or great arteries is indicative for said subject being at increased risk of developing or suffering from CHD.

The present invention also relates to a method for diagnosing in vivo CAVD in a subject, the method comprising,
(i) administering a substance to said subject, and
(ii) detecting if said substance accumulates in the leaflets of the aortic valve,
wherein the level of accumulation of said substance in the leaflets of the aortic valve is indicative for said subject being at increased risk of developing or suffering from CAVD.

Accordingly, the present invention relates in one aspect to a method for diagnosing in vivo CHD in a subject, the method comprising,
(i) administering a substance to said subject, and
(ii) detecting if said substance accumulates in the vessels or vessel walls of the coronary arteries and/or great arteries,
wherein the level of accumulation of said substance in the vessels or vessel walls of the coronary arteries and/or great arteries is indicative for said subject being at increased risk of developing or suffering from CHD.

Preferably, the accumulation of a radioactively labelled TLR3 specific ligand, antibody or nucleic acid probe, that is able to bind TLR3 in vivo, is measured in the leaflets of the aortic valve of a subject suspected being at risk of suffering of and/or developing and/or being predisposed to a disease associated with cardiovascular calcification, wherein said accumulation is indicative for said subject to develop and/or suffer from CAVD.

Accordingly, the present invention relates in one aspect to a radioactively labelled TLR3 specific ligand, antibody or nucleic acid probe, that is able to bind TLR3 in vivo for use in a method of in vivo diagnosis of CAVD in a subject, the method comprising:
(i) administering the radioactively labelled TLR3 specific ligand, antibody or nucleic acid probe, that is able to bind TLR3 in vivo to said subject, and
(ii) detecting if said radioactively labelled TLR3 specific ligand, antibody or nucleic acid probe, that is able to bind TLR3 in vivo accumulates in the leaflets of the aortic valve,
wherein the level of accumulation of said radioactively labelled TLR3 specific ligand, antibody or nucleic acid probe, that is able to bind TLR3 in vivo in the leaflets of the aortic valve is indicative for said subject being at increased risk of developing or suffering from CAVD.

The present invention also relates to a method for diagnosing in vivo CAVD in a subject, the method comprising,
(i) administering a radioactively labelled TLR3 specific ligand, antibody or nucleic acid probe, that is able to bind TLR3 in vivo to said subject, and
(ii) detecting if said radioactively labelled TLR3 specific ligand, antibody or nucleic acid probe, that is able to bind TLR3 in vivo accumulates in the leaflets of the aortic valve,
wherein the level of accumulation of said radioactively labelled TLR3 specific ligand, antibody or nucleic acid probe, that is able to bind TLR3 in vivo in the leaflets of the aortic valve is indicative for said subject being at increased risk of developing or suffering from CAVD.

Any of the TLR3 inhibitors disclosed herein may be radioactively labelled for use as a substance or in a substance in a method of in vivo diagnosis of a disease associated with cardiovascular calcification in a subject.

Any of the TLR3 inhibitors disclosed herein may be radioactively labelled for diagnosing in vivo a disease associated with cardiovascular calcification in a subject.

The present invention also relates to a kit comprising the binding molecule, the nucleic acid, the nucleic acid probe, the primer, the primer pair, the biotinylated primer, the SNP microarray, the single-stranded oligonucleotide probe or the substance according to any one of the products, uses or methods described herein.

The present invention also relates to a use of the kit as described herein for carrying out the methods or uses described herein.

A kit in the sense of the present invention may comprise any substance and/or composition useful to carry out the methods, uses or purpose-limited uses of the present invention. A kit may comprise any substance and/or composition disclosed in any of the herein disclosed embodiments. A kit may comprise a substance and/or a composition useful for assessing whether a subject is at increased risk of developing a disease associated with cardiovascular calcification. A kit may comprise one or more of a binding molecule, a nucleic acid, a nucleic acid probe, a primer, a primer pair, a biotinylated primer, a SNP microarray, a single-stranded oligonucleotide probe useful for any of the methods, uses or purpose-limited uses of the present invention. A kit may comprise a substance useful for in vivo diagnosis of a disease associated with cardiovascular calcification, such as CAVD and/or CHD, preferably CAVD in a subject.

A kit may preferably comprise a substance that is radioactively labelled and binds TLR3. A kit may preferably comprise a TLR3 specific, radioactively labelled ligand, antibody and/or nucleic acid probe. A kit may comprise one or more of a binding molecule, a nucleic acid, a nucleic acid probe, a primer, a primer pair, a biotinylated primer, a SNP microarray and/or a single-stranded oligonucleotide probe useful for carrying out the methods and/or purpose-limited uses of the present invention.

Accordingly, the present invention relates in one aspect to a kit comprising a substance that is radioactively labelled and binds TLR3.

Accordingly, the present invention relates in one aspect to a kit comprising a TLR3 specific, radioactively labelled ligand, antibody and/or nucleic acid probe.

Accordingly, the present invention relates in one aspect to a kit comprising a TLR3 inhibitor.

Accordingly, the present invention relates in one aspect to a kit comprising (R)-2-(3-Chloro-6-fluorobenzo [b] thiophene-2-carboxamido)-3-phenyl-propanoic acid.

Accordingly, the present invention relates in one aspect to a kit comprising an anti-TLR3 antibody CNTO4685 and/or CNTO5429.

A kit may also comprise comparative data to validate measurements in a subject suspected being at risk of suffering of and/or developing and/or being predisposed to a disease associated with cardiovascular calcification, such as CAVD and/or CHD, preferably CAVD. The comparative data may be a control sample obtained from a subject or a cohort of subjects that are not suspected being at risk of suffering of and/or developing and/or being predisposed to a disease associated with cardiovascular calcification, such as CAVD and/or CHD, preferably CAVD, and that are not diagnosed with said disease. The control sample may comprise nucleic acid sequences. The control sample may also comprise reference data that indicate a risk for a subject of developing and/or suffering from a disease associated with cardiovascular calcification that corresponds with a level of accumulation of a substance in cardiac tissue. The reference data are obtained by measuring physical samples correlating to different levels of accumulation comprised in the control sample prior or after the measurement of accumulation in a subject suspected being at risk of suffering of and/or developing and/or being predisposed to a disease associated with cardiovascular calcification.

Accordingly, the present invention relates in one aspect to a kit comprising comparative data to validate measurements in a subject suspected being at risk of suffering of and/or developing and/or being predisposed to a disease associated with cardiovascular calcification, such as CAVD and/or CHD, preferably CAVD.

Accordingly, the present invention relates in one aspect to a kit comprising comparative data to validate measurements in a subject suspected being at risk of suffering of and/or developing and/or being predisposed to a disease associated with cardiovascular calcification, such as CAVD and/or CHD, preferably CAVD, wherein the comparative data is a control sample obtained from a subject or a cohort of subjects that are not suspected being at risk of suffering of and/or developing and/or being predisposed to a disease associated with cardiovascular calcification, such as CAVD and/or CHD, preferably CAVD, and that are not diagnosed with said disease.

Accordingly, the present invention relates in one aspect to a kit comprising one or more control samples comprising reference data that indicate a risk for a subject of developing and/or suffering from a disease associated with cardiovascular calcification, wherein the risk corresponds with the reference data and the reference data is indicative for a level of accumulation of a substance in cardiac tissue.

Accordingly, the present invention relates in one aspect to a kit comprising one or more control samples comprising reference data that indicate a risk for a subject of developing and/or suffering from a disease associated with cardiovascular calcification, wherein the risk corresponds with the reference data and the reference data is indicative for a level of accumulation of a substance in cardiac tissue,
wherein reference data are obtained by measuring physical samples correlating to different levels of accumulation comprised in the control sample prior or after the measurement of accumulation in a subject suspected being at risk of suffering of and/or developing and/or being predisposed to a disease associated with cardiovascular calcification, such as CAVD and/or CHD, preferably CAVD.

In addition to above-mentioned components, a kit may further include instructions for using the components of the kit to practice the methods, uses and/or purpose-limited uses of the present invention. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. The instructions may be present as an electronic storage data file present on a suitable computer readable storage medium, e.g., CD-ROM, diskette, flash drive, etc. The actual instructions may not be present in the kit, but means for obtaining the instructions from a remote source, e.g., via the internet, are provided. An example of this is a kit that includes a web address where the instructions may be viewed and/or from which the instructions may be downloaded. As with the instructions, this means for obtaining the instructions is recorded on a suitable substrate.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

Thus, the terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means that specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

The term “a” as used herein refers to “one or more”, unless indicated otherwise herein.

As used herein the term “about” refers to +10%.

The terms “TLR3 inhibitor” and “inhibitor of TLR3” are used herein interchangeably.

As used herein the term “healthy” refers to the common understanding of the term in the art, i.e. a physical status of a subject being absent of sickness and symptoms thereof. The term “healthy” thus comprises the physical status of a subject being absent of major conditions or symptoms or sickness that affect to a degree that the regular daily life of said subject is significantly impaired.

As used herein, the term “early stage” refers in general to the early stage of a disease, such as a disease associated with cardiovascular calcification. Accordingly, early stage may refer to the stage of a disease before symptoms develop. Early stage can also refer to the stage of a disease when symptoms, e.g. mild symptoms, start to develop. Early stage can also refer to the stage of a disease in which diagnosis is possible, such as starting from the timepoint of the earliest possible diagnosis and thereafter. Early stage may also refer to a stage of a disease where a risk of developing or suffering from said disease can be assessed, wherein a prophylactic treatment may be desired. For example, a situation where a subject has an increased risk of developing a disease associated with cardiovascular calcification may be referred to as early stage of said disease. In the context of products, treatments, methods, or uses provided herein, a disease may preferably be at an early stage. A non-limiting example of an early stage disease in the sense of the present invention is (aortic) sclerosis (particularly in CAVD), which when untreated can develop to aortic stenosis. An early stage disease in the context of the present invention is preferably early stage CAVD and/or CHD.

### BRIEF DESCRIPTION OF THE FIGURES

FIG. 1 shows that aged Tlr3−/− mice are protected from CAVD. (a) Murine ascending aortas, descending aortas, subclavian arteries, carotid arteries and femoral arteries analyzed for Tlr3 expression. Healthy human aortic valves (upper panel) and valvular interstitial cells from human aortic valves (lower panel) analyzed for TLR3 (( )stained TLR3 (red) and nuclear DAPI staining (blue), scale=50 μm). n=3 per group. (b) Immunoblot analysis of human aortic valve specimen from CAVD patients and healthy controls for TLR3 expression. n=3 per group. (c) IFN-β expression measured via RT-PCR in CAVD samples and controls. n=9-10 per group. (d) TLR3 expression with increasing passage of VICs analyzed via immunoblot. n=2 independent western blot experiments. (e) Representative images from murine aortic valves from newborn, juvenile (3 weeks), adult (12 weeks) and aged (18 months) (stained TLR3 (red), and nuclear DAPI staining (blue, IF-scale=50 μm, H.E.-scale=250 μm) analyzed for (f) Tlr3 expression (n=5-6 animals per group) and (g) leaflet area (n=5-6 animals per group) and (h) leaflet thickness (n=5-6 animals per group). (i) Aortic valves from adult wild type mice (12 weeks), aged wild type mice (18 months) and aged Tlr3^{-/-} (18 months) analyzed morphologically via histological stainings and micro CT (scale=500 μm). (j) Aortic valve leaflet thickness measured in H.E. sections (n=6 animals per group). (k) Calcified area of aortic valve leaflets analyzed using von Kossa staining (n=8-12 animals per group). (l) Aortic valve leaflet thickness measured via micro CT (n=6-12 leaflets per group). (m) Echocardiographic assessment of aortic valves (AoV) from adult wild type mice, aged wild type mice and aged Tlr3^{-/-} measuring (n) leaflet thickness (n=5-10 animals per group) (o) mean pressure gradients (n=5-10 animals per group) and (p) transvalvular velocities (n=5-10 animals per group).

All data are presented as mean±SEM. Statistical comparisons: (C) 2-tailed unpaired t-test, ** P<0.01. (F-P) One-way ANOVA with Tukey post hoc test: * P<0.05, ** P<0.01, *** P<0.001, **** P<0.0001).

FIG. 2 shows that Tlr3-deficiency protects mice from hyperlipidemia-induced CAVD. (a) Aortic valves from 12-week-old wild-type mice of ApoE^{-/-} mice fed with HFD for 3 months stained for TLR3 expression (stained TLR3 (red), and nuclear DAPI staining (blue); scale=40 μm) and (b) Quantification of TLR3-expression on murine aortic valves (n=4-5 animals per group). (c) 12-week-old ApoE^{-/-} and ApoE^{-/-}/Tlr3^{-/-} (n=6-22 animals per group) double knockout mice fed with HFD for 3 months and analyzed for serum cholesterol, (d) serum triglyceride and (e) weight. (f) Aortic valves of 12-week-old ApoE^{-/-} and ApoE^{-/-}/Tlr3^{-/-} double knockout mice fed with HFD for 3 months analyzed morphologically. (g) Murine aortic valves analyzed for leaflet thickness in H.E. staining (scale=250 μm; n=6 animals per group). (h) Calcified area of murine aortic valves measured using von Kossa staining (scale=250 μm; n=3 animals per group). (i) Oilred O positive area within the aortic valve (scale=250 μm; 4-6 animals per group). (j) Aortic valves of 12-week-old ApoE^{-/-} and ApoE^{-/-}/Tlr3^{-/-} double knockout mice fed with HFD for 3 months analyzed for hemodynamics via transthoracic echocardiography assessing (k) leaflet thickness (n=11-20 animals per group) (l) aortic valve diameter (n=12-23 animals per group) (m) aortic valve opening (n=12-21 animals per group).

Statistical comparisons: (b,c,d,e) 2-tailed unpaired t-test, ** P<0.01. (g,h,i,k,l,m) One-way ANOVA with Tukey post hoc test: * P<0.05, ** P<0.01, *** P<0.001, **** P<0.0001).

FIG. 3 shows that biglycan serves as an endogenous ligand of TLR3. (a) TLR3 expression analysis of hVICs subjected to stretch (15%) for either 6 h or 24 h (n=3 samples per group, measured in duplicates)

(b) TLR3, TRIF and TRAF6 expression with increasing passage of VICs analyzed via immunoblot in n=2 independent western blot experiments.

(c) HEK293 hTLR3 reporter cells treated with either Poly(I:C) (20 μg/ml) or supernatant from stretched VICs for 24 h.

(d) HEK293 hTLR3 reporter cells treated with Poly(I:C) (20 μg/ml) and increasing concentrations of biglycan (5 μg/ml, 10 μg/ml, 20 μg/ml), (n=3 samples per group, measured in duplicates).

(e) HEK293 hTLR3 reporter cells treated with Poly(I:C) or biglycan (both 5 μg/ml) for 6 h in the presence of (R)-2-(3-Chloro-6-fluorobenzo [b] thiophene-2-carboxamido)-3-phenylpropanoic acid, a dsRNA/TLR3 complex inhibitor (n=3 samples per group, measured in duplicates).

(f) Binding experiments with the recombinant human TLR3 ectodomain and biglycan (size-exclusion chromatography and subsequent immunoblot analysis).

(g) After a Co-Immunoprecipitation of purified TLR3 ectodomain and purified BGN both proteins were detected on immunoblots (n=2).

(h) Illustration of the dimer binding mode. The top view (left panel) shows the location of the 2-fold axes perpendicular to the projection plane with dyad symbols, and the right panel shows the assembly of the model maintaining the 2-fold symmetry (vertical line (red)). The docked, refined, and optimized model (i) is presented as ribbons surrounded by a macro shape which includes the forests core residues of all glycan decorations, none of which cause any steric clashes.

(j) HEK293 hTLR3 reporter cells treated with biglycan in combination with endocytosis inhibitor dynasore (n=3 samples per group, measured in duplicates).

(k) HEK293 hTLR3 reporter cells treated with full-length biglycan or biglycan core protein (rh) (n=3 samples per group, measured in duplicates).

(l) Immunoblot analysis of VICs extracted from human aortic valves of CAVD patients and healthy controls for BGN and XYLT1 expression (n=3 per group)

(m) HEK293 hTLR3 reporter cells treated with supernatants from stretched VICs with prior siRNA knockdown od XYLT1 or control (scRNA) (n=3 samples per group, measured in duplicates).

Statistical comparisons: (b,c,g,h,j) One-way ANOVA with Bonferroni post hoc test: * P<0.05, ** P<0.01, **** P<0.0001)

FIG. 4 shows that TLR3 induces calcification via IFN I signaling.

(a) VICs treated with Poly(I:C) for 2, 4, 6, 24 and 48 h and subsequent RT-qPCR analysis of TLR3 (n=3 samples per group) and

(b) IFN-β (n=3 samples per group measured in duplicates). (c) RT-PCR analysis of human wild-type dermal fibroblasts, Tlr3^{-/-} CRISPR fibroblasts or empty vector control fibroblasts (CTR) treated with Poly(I:C) or BGN (both 5 μg/ml) for 6 h and the analyzed for TLR3 expression. (n=3 samples per group measured in duplicates).

(d) RT-PCR analysis of human wild-type dermal fibroblasts, Tlr3^{-/-} CRISPR fibroblasts or empty vector control fibroblasts (CTR) treated with Poly(I:C) or BGN (both 5 μg/ml) for 6 h and then analyzed for IFN-β expression. (n=3 samples per group measured in duplicates).

(e) Immunoblot analysis of human wild-type dermal fibroblasts and Tlr3^{-/-} CRISPR fibroblasts treated with Poly(I:C) or BGN (both 5 μg/ml) for 6 h.

(f) Schematic illustration of the involved TLR3-RUNX2 pathway.

(g) Expression of the osteoblastic transcription factor RUNX2 after Poly(I:C) treatment (20 μg/mL) in the presence of either LY294002 (10 μM), an IRF3 inhibitor, or a specific IFNAR1 blocking antibody (4 μg/ml). GAPDH served as loading control (n=2 independent western blot experiments).

(h) Histological evaluation of aortic valves from wild-type, Tlr3^{-/-}, Bgn^{-/-} or Ifnar1^{-/-} animals subjected to high-fat diet for 4 weeks analyzed via H.E. staining and von Kossa staining.

(i) Gain of weight after 4 months of high-fat diet of wild-type, Tlr3^{-/-}, Bgn^{-/-} or Ifnar1^{-/-} animals (n=5-8 animals per group).

(j) Serum triglyceride levels after 4 months of high-fat diet of wild-type, Tlr3^{-/-}, Bgn^{-/-} or Ifnar1^{-/-} animals (n=5-8 animals per group).

(k) Analysis of aortic valve leaflet thickness in wild-type, Tlr3^{-/-}, Bgn^{-/-} or Ifnar1^{-/-} animals after 4 months of high-fat diet (n=5-8 animals per group).

(l) Quantification of aortic valve calcification in wild-type, Tlr3^{-/-}, Bgn^{-/-} or Ifnar1^{-/-} animals after 4 months of high-fat diet (n=5-8 animals per group).

(m) Assessment of aortic valve function after 4 months of high-fat diet in wild-type, Tlr3^{-/-}, Bgn^{-/-} or Ifnar1^{-/-} animals via transthoracic echocardiography (n=5-8 animals per group).

(n) Analysis of aortic valve leaflet thickness in wild-type, Tlr3^{-/-}, Bgn^{-/-} or Ifnar1^{-/-} animals after 4 months of high-fat diet (n=5-8 animals per group).

(o) Aortic valve opening in wild-type, Tlr3^{-/-}, Bgn^{-/-} or Ifnar1^{-/-} animals after 4 months of high-fat diet (n=5-8 animals per group).

(p) Mean transvalvular pressure gradient in wild-type, Tlr3^{-/-}, Bgn^{-/-} or Ifnar1^{-/-} animals after 4 months of high-fat diet (n=5-8 animals per group).

(q) Transvalvular peak velocities in wild-type, Tlr3^{-/-}, Bgn^{-/-} or Ifnar1^{-/-} animals after 4 months of high-fat diet (n=5-8 animals per group).

All data are presented as mean±SEM.

Statistical comparisons: (a), (b), (c), (d), (j), (k), (l), (n), (o), (p), (q) One-way ANOVA with Tukey post hoc test: * P<0.05, ** P<0.01, *** P<0.05, **** P<0.0001)

FIG. 5 shows that TLR3 promotes calcification and bone development in vivo

(a) Numbers of the at least four-fold change differentially expressed genes in human VICs with or without Poly(I:C) treatment (20 μg/mL) for 72 h and human osteoblasts.

(b) Expression profile of genes involved in calcification.

(c) Gene enrichment set analysis of VICs treated with Poly(I:C) (20 μg/mL) for 72 h evaluating regulation of genes involved in calcification.

(d) VICs cultured with osteoblastic medium in the presence of TLR3 agonist Poly(I:C) (20 μg/mL) or inhibitor (26 μM), fixated and stained with Alizarin Red S (scale=40 μm).

(e) Quantification of calcific nodules (n=3 samples per group, measured in triplicates).

(f) HEK293 hTLR3 reporter cells treated with TLR3 inhibitor (26 μM) or Poly(I:C) (20 μg/ml) (n=3 samples per group, measured in duplicates)

(g) Zebrafish treated with VitD3 and VitD3 in the presence of TLR3 inhibitor with subsequent quantification of calcification of

(h) the opercle (n=10-19 animals per group, scale=100 μm)

(i) all bone structures (n=6 animals per group, scale=100 μm).

(j) Femurs from 12-week-old wild-type, Tlr3^{-/-}, Bgn^{-/-} or Ifnar1^{-/-} mice (n=5-8 animals per group) analyzed morphologically via micro CT for

(k) bone volume,

(l) bone density,

(m) trabecular distance,

(n) trabecular networks

(o) number of trabecles/volume and

(p) trabecular thickness (n=5-8 animals per group).

All data are presented as mean±SEM.

Statistical comparisons: (e), (f), (h), (i), (k), (l), (m), (n), (o), (p) One-way ANOVA with Tukey post hoc test: * P<0.05, ** P<0.01, *** P<0.001, **** P<0.0001)

FIG. 6 shows the association of TLR3 pathway variants in patients with aortic stenosis. (a) Association with aortic stenosis in a meta-analysis of UK Biobank and GERA cohorts of variants within 50 kb of 8 autosomal genes. Variants are independent (r²<0.01) and have an association of 1) p≤1×10⁻³ or 2) p≤0.05 and odds ratio ≥2. FIG. 12 includes associations irrespective of linkage disequilibrium. (b) Mendelian randomization for the association with aortic stenosis of the six variant genetic instruments for XYLT1 expression in the aorta. Points represent the association of each variant with aortic stenosis versus its association with XYLT1 expression. The trendline and shaded region are the inverse-variance weighted estimate and 95% confidence interval, and accounts for genetic correlation between variants in the instrument (r²≥0.23). (c) P values for the association of variants in the XYLT1 expression instrument with gene expression in the aorta and epigenetic modification. P values for epigenetic modification include associations of variants in high linkage disequilibrium (r²≥0.9 in European populations), with the colour of each cell representing the most significant association.

Abbreviations: GERA, Genetic Epidemiology Research on Adult Health and Aging.

FIG. 7 shows TLR3 reporter assays for ligand identification. (a) Immunoblot for TLR3 expression of hVICs treated putative TLR3 ligands biglycan (20 μg/ml), oxLDL (20 μg/ml) and Lp(a) (20 μg/ml) for either 24 h or 48 h (n=2)

HEK293 hTLR3 reporter cells treated with Poly(I:C) (20 μg/ml) and the putative TLR3 ligands (b) Lp(a), (c) oxLDL, (d) oxPAPC, (e) POVPc, (f) ALDOPc, (g) PAEZEPc, (h) PGPc, (i) decorin, (j) aggrecan, and (k) fibromodulin at increasing concentrations for 24 h (n=3 samples per group measured in duplicates). All data are presented as mean±SEM.

FIG. 8 BGN characterization, in silico modeling of BGN and TLR3. (a) Prediction of potential binding regions on BGN and (b) TLR3-ECD by the ODA method, indicated by the (red-colored) arrows in the CPK model. The spheres (blue) show the expected location of the N-terminal chondroitin sulfate modifications of BGN.

(c) Comparison of BGN and recombinant rhBGN treated with chondroitinase ABC and PNGase analyzed by SDS-PAGE analysis and subsequent staining with Coomassie Brilliant Blue.

(d) Human VICs transfected with XYLT siRNA or scrRNA for 24 h followed by RT-PCR analysis for XYLT1, (e) TLR3 and (f) IFNß (n=3 samples per group, measured in duplicates).

(g) HEK293 hTLR3 reporter cells subjected to stretch with prior knockdown of XYLT1 (n=3 samples per group measured in duplicates).

All data are presented as mean±SEM.

Statistical comparisons: (d), (e), (f), (g) 2-tailed unpaired t-test, **** P<0.0001. (g) One-way ANOVA with Tukey post hoc test: **** P<0.0001

FIG. 9 shows RNA Sequencing comparing the expression profiles of hVICs, human dermal fibroblasts and human osteoblasts.

For the analysis hVICs and human dermal fibroblasts (hdFB) were treated with Poly(I:C) (20 μl/ml) for 72 h. Treated samples were compared to untreated controls and human osteoblasts (samples were measured in triplicates).

(a) Principal component analysis of analyzed samples.

(b) Comparison of the genes that were up- or downregulated between all groups and intersection of treated and untreated hVICs and hdFB.

(c) Heat map with all groups of genes that were at least four-fold differentially expressed (red=upregulated, blue=downreulated, black=not regulated).

(d) Intersection plot to show the intersections of the genes in between the indicated groups.

(e) Volcano plot and gene set enrichments of treated hVICs compared to untreated hVICs and treated hdFB compared to untreated hdFB.

FIG. 10 shows Validation of specificity and linearity of antibodies.

Immunoblots of human dermal fibroblasts transfected with scr RNA or (a) STAT3, (b) JAK1, (c) BGN, (d) RUNX2 and (e) TLR3 siRNA for 24 h. Detection followed with the corresponding antibodies respectively.

FIG. 11 shows that TLR3 induces calcific gene expression.

(a) Gene enrichment set analysis of hVICs treated with Poly(I:C) for (20 μg/ml) for 72 h evaluating the regulation of genes involved in calcification in CAVD.

(b) Numbers and expression profiles of at least four-fold change differentially expressed genes in hVICs with or without Poly(I:C) (20 μg/ml) treatment for 72 h and human osteoblasts. Significantly over-expressed gene ontology terms and pathway among the commonly upregulated genes were indicated (red=upregulated, blue=downreulated, black=not regulated).

FIG. 12 shows Associations* with aortic stenosis in a meta-analysis of UK Biobank and GERA cohorts of variants within 50 kb of 8 autosomal genes. Abbreviations: GERA, Genetic Epidemiology Research on Adult Health and Aging; UKB, UK Biobank. 1 In linkage disequilibrium with rs569915578 (r²=1). 2 In linkage disequilibrium with rs62033189 (r²≥0.098). * Associations with either 1) p≤1×10⁻³ or 2) p≤0.05 and odds ratio ≥2 are provided.

FIG. 13 shows mendelian randomization for aortic stenosis of XYLT1 expression in the aorta.

FIG. 14 shows Associations with XYLT1 expression in the aorta and with aortic stenosis of variants in the Mendelian randomization. Estimates for aortic stenosis were from the meta-analysis of the GERA and UK Biobank cohorts.

Abbreviations: AS, aortic stenosis; GERA, Genetic Epidemiology Research on Adult Health and Aging.

FIG. 15 shows schematics of TLR activation via BGN, of an association study to identify genetic predispositions and of an evolutionary conserved function of TLR3 in osteogenesis.

### EXAMPLES

### Methods

### Patient Samples

Ethical permission for the use of human material has been obtained from the Medical University of Innsbruck (AN2014-026 340/4.34). After informed consent, valve tissue was obtained from (a) patients undergoing heart valve replacement because of CAVD and (b) patients undergoing heart transplantation (HTX) for other reasons than valve pathology (controls). Tissue was obtained in the operating room under sterile conditions and placed in sterile M199 culture medium (Life Technologies, Carlsbad, CA) supplemented with 10% fetal calve serum (FCS), penicillin, streptomycin and L-Glutamine. Samples were transported to the laboratory on ice and processed immediately thereafter.

### Cell Isolation and Culture

Valvular interstitial cells from patients undergoing aortic valve replacement or cardiac transplantation were isolated via collagen digestion as described previously⁶. Valves were rinsed in EBSS and subsequently subjected to collagenase digestion (Sigma, St. Louis, MO; 2.5 mg/mL in M199) for 30 minutes at 37° C. Thereafter, tissue samples were vortexed and supernatant containing endothelial cells was removed. Again, collagenase was added to the remaining valve tissue (1 mg/mL) for 3 h at 37° C. For mechanical disintegration of the tissue, vortexing and repeated aspiration via a pipette was performed. The supernatant was transferred into a new tube and subjected to centrifugation (300 g for 8 minutes at 4° C.). Subsequently, cells were resuspended in M199 medium containing 10% fetal calve serum, penicillin, streptomycin, L-Glutamine and amphotericin B (2.5 mg/L).

SV40 immortalized mouse embryonic fibroblasts (MEFs) were cultured in DMEM with 4.5 g/L glucose (Lonza, Basel, Switzerland), 10% FCS (Sigma, St. Louis, MO), penicillin, streptomycin, L-Glutamine and amphotericin B (2.5 mg/L).

Human osteoblasts were purchased (Promocell, Heidelberg, Germany) and cultivated in DMEM with 4.5 g/L glucose (Lonza, Basel, Switzerland), 10% FCS (Sigma, St. Louis, MO), penicillin, streptomycin, L-Glutamine and amphotericin B (2.5 mg/L).

Human dermal fibroblasts WT and Tlr3^{-/-} were kindly provided by the Jean-Laurent Casanova group and cultivated in DMEM with 4.5 g/L glucose (Lonza, Basel, Switzerland), 10% FCS (Sigma, St. Louis, MO), penicillin, streptomycin and L-Glutamine. Human dermal fibroblasts CTR cells were provided by the Villunger group and cultivated in DMEM with 4.5 g/L glucose (Lonza, Basel, Switzerland), 10% FCS (Sigma, St. Louis, MO), penicillin, streptomycin and L-Glutamine.

All cells were cultivated under standard conditions (37° C., supplied with 5% CO2). Passaging of the cells was performed at 70-90% confluency, and cells were only used until passage 7 as described previously⁶. Polyinosinic: polycytidylic acid (Poly(I:C); Invivogen, San Diego, CA) was used to stimulate cells at a concentration of either 20 μg/mL or 5 μg/ml, depending on the assay. For blockade of TLR3, the commercially available dsRNA/TLR3 inhibitor ((R)-2-(3-Chloro-6-fluorobenzo [b] thiophene-2-carboxamido)-3-phenyl-propanoic acid) was used (Merck, Darmstadt, Germany) at a concentration of 1 μg/ml as described previously^{1,7}. LY294002 (Invivogen, San Diego, CA), a PI3K inhibitor, was used at a concentration of 10 μM. For blockade of IFNAR1, a specific blocking antibody (Invitrogen, Carlsbad, CA) was used at a concentration of 4 μg/ml. A stable HEK (human embryonic kidney) reporter cell line (TLR3/ISRE LUCPorter; Imgenex, USA) was purchased for luminometer assay experiments. Cells were cultivated according to the manufacturer's protocol in DMEM with 4.5 g/L glucose (Lonza, Switzerland), 10% FCS (Sigma, St. Louis, MO), penicillin/streptomycin, and 4 mM L-glutamine and 3 mg/mL puromycin (Gibco, USA) as selection agent. Reporter experiments were performed as described previously⁸. For the protein expression SF21 cells were purchased at ThermoFisher (#11497013) and cultivated in Gibco™ Sf-900™ SFM supplemented with penicillin, streptomycin and L-Glutamine. In vitro stretch was applied using a Flexcell bioreactor (Flexcell International Corporation, Burlington, NC) applying 15% cyclic strain as described previously⁹.

### Transfection

The siRNA knock down was performed using the DharmaFECT1 Transfection Reagent (horizon, #T-2005-01). The transfection was performed according to the guidelines provided by Horizon™. The siRNAs that were used were purchased through Santa Cruz Biotechnology.

| | |
|---|---|
| XylT-I siRNA (h) | sc-61817, Lot # K2907 |
| RUNX2 siRNA (h) | sc-37145, Lot # E1515 |
| Biglycan siRNA (h) | sc-43633, Lot # B2806 |
| TLR3 siRNA (h) | sc-36685, Lot # B2718 |
| Stat3 siRNA (h) | Sc-29493, Lot # H0917 |

As a negative control AllStars Neg. Control siRNA (20 nmol) (Quiagen, #1027281) was used at the same conditions.

### Calcification Assay

For functional evaluation of osteoblastic activity, VICs were subjected to a calcification assay. Cells were cultured in osteogenic medium including 10 mmol/L beta-glycerophosphate, 10 nmol/L vitamin D3, and 10 nmol/L dexamethasone as described previously⁴. The medium was changed every three days. Subsequently, supernatant was analyzed for alkaline phosphatase activity using the commercially available alkaline phosphatase assay kit (Abcam, Cambridge, UK). After careful rinsing with PBS, cells were fixed with 4% paraformaldehyde, washed with distilled water and Alizarin Red S staining solution (Alfa Aesar, Haverhill, MA) was added upon the fixed cells for 20 minutes at room temperature (RT). Imaging of the staining was performed using a Zeiss Axioplan 2 microscope (Zeiss, Oberkochen, Germany). Images were processed using Adobe Photoshop CS5.1 for Mac (Adobe Systems Inc., San Jose, CA, USA) and analyzed with ImageJ software (National Institutes of Health, Bethesda, MA).

### RNA Sequencing Analysis

Total-RNA was isolated from five samples (VICs, cultured in calcification media and treated either 72 h with 20 μg/ml Poly(I:C), untreated controls, as well as human osteoblasts) and submitted to transcriptome analysis for the purpose of gene-expression profiling. For the RNA sequencing analysis in FIG. 5 the 2100 Bioanalyzer and RNA 6000 Nano LabChip kits (Agilent Technologies) was used to check for RNA integrity (all RINs >9). The TruSeq® Stranded mRNA HT technology was used for library preparation according to manufacturer's protocol and Illumina NextSeq® 500 1×75 bp single end sequencing was performed at the IMGM® laboratories (Martinsried, Germany). The RNA sequencing analysis in Figure S3 was performed at Novogene (UK) Co. using the NovaSeq 600 PE150.

All primary sequencing analyses were performed using CLC Genomics Workbench (9.5.3) including sequence QC and mapping to the human reference genome (GRCh38.p7). Differentially expression analyses were performed on raw count data and using an exact test based on negative binomial distribution (edgeR version 3.4.0) 10. P-values were adjusted according to the Benjamini-Hochberg method based on the false discovery rate (FDR). Only genes with at least four-fold change were considered. Over representation analysis for gene ontology and pathways on differentially expressed genes were performed using DAVID (6.8) 11 and ConsensusPathDB¹². Gene set enrichment analysis on log 2-fold change pre-ranked data was performed using GSEA software¹³ and gene sets for biological hallmark processes and pathways (MSigDB), calcification signature as derived from respective literature, and signatures of differentially expressed genes for calcific aortic valve stenosis versus control¹⁴ (reanalyzed data from GSE12644 using R package limma¹⁵ including genes with more than two-fold change and FDR <0.05). Heat maps were generated using Genesis version 1.8.116 for log 2-fold change data.

### RT-PCR

RT-PCR was performed as described previously¹⁷. Total RNA was extracted from homogenized tissue or cell lysates using the Total RNA Miniprep Kit (New England BioLabs, Frankfurt, Germany) according to the manufacturer's instructions. We performed the PCR reaction in a final volume of 12.5 μL containing 2.5 μL of cDNA, 6.25 μL of Master Mix, 1 μL of fluorogenic hybridization probe, 10 μM of primer and 2.75 μL of distilled water. Amplification was performed in a two-step PCR (40 cycles; 15-s denaturation step 1 at 95° C. for 1 min annealing/extension step at 60° C. for 30 sec). We normalized specific gene expression to the housekeeping gene GAPDH given by the formula 2−ΔCt. The result for the relative gene expression was calculated by the 2-DDCt method. Mean Ct values were calculated from double determinations and samples were considered negative if the Ct values exceeded 4017. Primers were designed using the Primer3Plus Software and obtained by Microsynth AG (Balgach, Switzerland). Primer sequences are listed in Table 1.

TABLE 1
Primer sequences

| | |
|---|---|
| human TLR3 | FW: 5′-AGGAAAGGCTAGCAGTCATCC-3′ (SEQ ID NO: 1) |
| | RV: 5′-TAACAGTGCACTTGGTGGTG-3′ (SEQ ID NO: 2) |
| human IFNb | FW: 5′-TGAGAACCTCCTGGCTAATGTC-3′ (SEQ ID NO: 3) |
| | RV: 5′-TTTTCAGGTGCAGACTGCTC-3′ (SEQ ID NO: 4) |
| human XYLT1 | FW: 5′-GCTGCTGATGCCTGAGAAGGT-3′ (SEQ ID NO: 5) |
| | RV: 5′-GACAAAGGCGATTCTGACCGG-3′ (SEQ ID NO: 6) |
| mouse TLR3 | FW: 5′-TTGTCTTCTGCACGAACCTG-3′ (SEQ ID NO: 7) |
| | RV: 5′-CCGTTCCCAACTTTGTAGATG-3′ (SEQ ID NO: 8) |

### Lysate Preparation and Immunoblotting

Protein from tissue or cell lysates was extracted using a Radioimmunoprecipitation Assay Lysis Puffer (150 mM NaCl, 1% NP-40, 0.5% Sodium Deoxycholate, 0.1% SDS and 50 mM Tris (pH 8,0)). For the extraction of nuclear or cytosolic proteins the NE-PER™ Nuclear and Cytoplasmic Extraction Reagents Kit (ThermoScientific, #78835) was used.

Protein was thereafter separated on SDS-polyacrylamide gels of different percentages and transferred to nitrocellulose membranes as described previously¹⁸. After blocking with either 5% BSA in TBS 0.1% Tween, 5% milk in TBS 0.1% Tween or StartingBlock™ (TBS) Blocking buffer (ThermoScientific, #37542) membranes were incubated with primary antibodies listed in Table 2.

TABLE 2
Antibodies used for Western Blot analysis.

| | |
|---|---|
| TLR3 | Cell Signaling Technologies, Danvers, MA; #6961 |
| TRIF | Cell Signaling Technologies, Danvers, MA; #4596 |
| TRAF6 | Abcam, Cambridge, UK; ab137452 |
| IRF3 | Cell Signaling Technologies, Danvers, MA; #4302 |
| pIRF3 | Cell Signaling Technologies, Danvers, MA; #29047 |
| IFNAR1 | GeneTex, #N1N3 |
| JAK1 | Cell Signaling Technologies, Danvers, MA; #3332 |
| pJAK1 | Cell Signaling Technologies, Danvers, MA; #3331 |
| STAT3 | Cell Signaling Technologies, Danvers, MA; #9132 |
| pSTAT3 | Cell Signaling Technologies, Danvers, MA; #9145 |
| RUNX2 | Abcam, Cambridge, UK; ab23981 |
| GAPDH (6C5) | Invitrogen, Carlsbad, CA; #AM4300 |
| TBP | Cell Signaling Technologies, Danvers, MA; #8515 |
| Actin | Sigma, St. Louis, MO; A2066 |
| XYLT1 | Novusbio, #NBP1-91245 |
| BGN | R&D Systems, Minneapolis, #AF2667 |

After incubation with the secondary antibody, chemiluminescence was detected by either adding Clarity™ Western ECL Substrate (Bio-Rad, #170-5060) or ECL™ Prime Western Blotting Detection Reagents (Amersham™, #RPN2232).

### Histology

Tissue samples were either fixed in 4% paraformaldehyde buffered in PBS (0.1M) and subsequently embedded in paraffin or in OCT compound (TISSUE-TEK®, Sakura Finetek, Netherlands) for cryosections. 5 μm tissue sections were stained using a standard hematoxylin and eosin staining protocol (Thermo Fisher Scientific, Waltham, MA). Additional sections were stained with a silver staining kit according to von Kossa (Merck, Darmstadt, Germany) and Oil-Red-O (Sigma, St. Louis, MO).

Bone tissue was decalcified with ethylenediaminetetraacetic acid (EDTA) for three weeks at room temperature prior to tissue processing. Immunohistochemistry was performed as described previously¹⁹. Paraffin-embedded sections underwent heat mediated antigen retrieval using a sodium-citrate buffer (10 mM sodium-citrate, 0.05% Tween 20, pH 6.0). Subsequently, blocking of the sections for 30 minutes using 5% serum in PBS was performed. The following primary antibodies were used for staining (Table 3):

TABLE 3
Antibodies used for immunofluorescence staining

| | |
|---|---|
| TLR3 | abcam, Cambridge, UK; ab62566 |
| RUNX2 | abcam, Cambridge, UK; ab23981 |

Primary antibodies were incubated overnight at 4° C. Alexa Fluor 568 and Alexa Fluor 488 conjugated IgG antibodies (Life Technologies, Carlsbad, CA) served as secondary antibodies, whereas 40,6-diamidino-2-phenylindole (DAPI; Life Technologies, Carlsbad, CA) was used for nuclear counterstaining. Light microscopy was performed using a Zeiss Axioplan 2 microscope (Zeiss, Oberkochen, Germany), immunofluorescence was assessed using a Leica SP5 confocal microscope (Leica, Wetzlar, Germany). Images were analyzed using ImageJ software (National Institutes of Health) and processed with Adobe Photoshop CS5.1 for Mac (Adobe Systems Inc., San Jose, CA, USA).

### Mouse Models

Ethical permission for all animal experiments has been obtained (BMWFW 66.011/0152-WF/V/3b/2014 and BMWF-66.011/0101-V/3b/2018). We performed the experiments conformed to the ‘Guide for the Care and Use of Laboratory Animals’ published by the US National Institutes of Health (NIH Publication No. 85-23, 1996, revised 2011; available from: www.nap.edu/catalog/5140.html). All measurements and analyses were performed in a blinded fashion.

Experiments were performed in C57BL/6N mice (Charles River Laboratories, Wilmington, MA) and Tlr3^{-/-} mice (C57BL/6N background). Euthanasia was performed by cervical dislocation in anesthesia. For our hyperlipidaemic model, Tlr3^{-/-} mice were crossed with ApoE^{-/-} mice obtained from Jackson Laboratories (Bar Harbor, ME). Generated ApoE^{-/-}/Tlr3^{-/-} double-knockout mice were set on western type diet (Ssniff, Soest, Germany) for 3 months. For induction of aortic valve stenosis, a proatherogenic High Fat/High Carbohydrate (HF/HC) diet without added cholesterol (#F3282, BioServ, Frenchland, NJ) was used as described previously²⁰. C57BL/6N, Tlr3^{-/-}, Bgn^{-/-} (C3.129S4(B6)-Bgntm1Mfy/Mmmh; MMRRC) and Ifnar1^{-/-} (B6.129S2-Ifnar1tm1Agt/Mmjax; Jackson Laboratory, Bar Harbor, ME) mice were fed a HF/HC diet ad libitum for 4 months.

### Echocardiography

Transthoracic echocardiography measurements were performed as described previously^{17,21}. Mice were subjected to anaesthesia with Isoflurane 1.5% (AbbVie, Vienna, Austria) and 98.5% 02 and placed on a warming pad at 37.5° C. before and during the procedure. To conduct ultrasound examination, the Vevo 1100 imaging system and Visual Sonics Software Vevo Lab 1.7.1 (Visual Sonics, Toronto, Canada) were used. Measurements in parasternal longitudinal axis (PSLAX) were performed with a MS400 (18-38 MHz) transducer. Aortic valve opening diameter and aortic valve leaflet thickness were determined in M-Mode by freehand measurement. All measurements and analyses were performed in a blinded fashion.

### Micro Computed Tomography Analysis

After fixation in 4% formaldehyde for 24 hours, hearts were incubated for 24 hours in an iodine-based contrast medium solution (Jopamiro 300 mg; BIPSO, Singen, Germany) prior to critical point drying. Femurs were fixed in 4% formaldehyde after detachment from the hip joint. Experiments were performed using a vivaCT 40 (Scanco Medical AG, Brüttisellen, Switzerland) using 1000 projections with 2048 samples and a 21.5 mm field of view (FOV) resulting in a 10.5 μm isotropic resolution per voxel. The tube settings were 45 kV voltage, 177 μA current with an integration time of 380 ms per projection.

For assessment of the thickness of the aortic wall and the aortic valve leaflets, a semi-automatic algorithm was used. Thickness was calculated as described previously²². For the assessment of bone morphology analysis was performed as described²³⁻²⁵. 3D image formation was performed using the manufacturer provided software tool.

### Zebrafish Model

Fish were maintained and raised under standard husbandry conditions. VitD3 treatments were performed as previously described²⁶ with some minor modifications. Briefly: 15 animals (5 dpf) were pooled per 6-well pate and incubated for 5 days at 28° C. in 4 ml E3 medium containing 0.1% DMSO and different compounds as indicated. Larvae were fixed in 4% PFA overnight. Final concentration of compounds was 5 μg/mL for TLR3-inhibitor (Merck, Darmstadt, Germany) and 200 ng/mL VitD3 (Sigma, St. Louis, MO). Dilutions were generated from stock solutions with 10 mg/mL TLR3-INH and 200 mg/mL VitD3 in DMSO. Solutions containing VitD3 were changed every day. All experiments were done in duplicates. Alizarin Red stains were performed as described previously²⁷ with some minor modifications. After staining larvae were transferred to 100% glycerol and imaged. Area measurements were carried out as described²⁸.

Whole area: For each larva three independent ventral view images were taken with a Leica MZ16FA using standardized stereomicroscope settings. After each image the larvae were embedded anew. Data quantification was done with ImageJ. Standard white balance was used for background subtraction. Image were converted to greyscale (8 bit) and threshold was set to 225. The stained area excluding the otoliths was measured. Data points shows mean area from the three images taker per embryo.

Opercula area: Larvae were imaged laterally from both sides using a standard microscope setting. Area measurements were performed as described above.

### Statistical Analysis

Results are presented as mean±standard error of the mean. Statistical comparisons between two groups were performed either by Student's t-test or Mann-Whitney test, whereas multiple groups were compared using one-way ANOVA with Tukey post hoc analysis for statistical significance. P-values <0.05 were considered statistically significant.

### Structure Analysis and Modelling

We selected a docking protocol that first docks the non-glycosylated proteins based on shape complementarity²⁹ or docked pre-aligned models based on symmetry considerations, followed by local refinement of the docked models³⁰. Docking results were then cross validated by adding the glycans at all consensus sites, assuring that the docked complex did allow for the presence of the N-linked glycan chains. The plausibility of the model was then assessed in view of known biological data.

huTLR3-ECD

The experimental high-resolution structure model of the TLR3 receptor ectodomain (ECD), PDB entry 1ziw³¹, was used as a template for modelling a glycosylated and completed human TLR3-ECD dimer model. All 15 experimentally verified glycosylation sites from 1ziw³², 2a0z³³, and 5gs0³⁴ were decorated with the first 8 carbohydrate core units (GlucNac₂(Fuc)Man₅) of human glycans in idealized geometry using the glycan building tools in COOT³⁵. Flexible glycan decorations are present in vivo and often only partially visible in electron density of X-ray structures, but they must be included in the model to assess where they occupy space and limit possible orientations³⁶. Loop 336-343 missing in 1ziw was completed based on humanized mouse TLR3 model 3ciy³². All TLR3 structures show identical crystallographic or non-crystallographic dimers indicating that the TLR3 receptor will likely bind biglycan as a dimer.

### BGN

The experimental structure model of the glycoprotein core of BGN, PDB entry 2ft3³⁷, was used as a template for modelling huBGN. BGN is an obligate biological dimer and shares 98% sequence identity for the mature protein. The 2ft3 structure includes one partially modelled glycan and starts at residue 24 of the mature peptide, and the N-terminal O-linked glycosaminoglycan decorations were removed by digestion with chondroitin ABC lyase and are thus absent from the crystal structure model. To produce a huBGN model for docking, following steps were taken: (i) humanize the sequence and locally minimize the mutated structure in ICMPro³⁸, and (ii) add the first residues of a human glycan (GlucNag₂(Fuc)Man₅) in idealized geometry to glycosylation sites N234 and N275 using COOT³⁵ in order to assess regions that need to be excluded from docking. No attempt was made to model the N-terminal glycosaminoglycan chains because they are not in the vicinity of predicted binding sites.

### Binding Site Prediction

Binding site predictions based on the Optimal Docking Areas (ODA) method³⁹ identifies the C-terminal of biglycan as the most likely interaction area (FIG. 4G), consistent with the fact that the, in structure models absent, N-terminal residues bearing the glycosaminoglycan decorations are not likely involved in direct protein-protein contacts (FIG. 4H). The BGN glycosylations are distal to the proposed binding region, and are likely not a hindrance for the formation of protein-protein contacts.

### Possible Binding Modes

Both TLR3 and BGN form obligate dimers which strongly suggest that they interact also as dimers in vivo and form either a (TLR3₂)(BC₂) complex between the respective dimers (binding mode A) or a (TLR3₂)(BC₂)₂ complex (mode B) where one biglycan dimer binds to each of the subunits of the TLR3 dimer. Mode A requires that collinearity of the dimer axes is maintained, thus effectively reducing the docking search space. The Euler axis for each dimer was computed from the Directional Cosine Matrix obtained by superposition of the models in COOT³⁵, with its location is given by half of the superposition translation vector. The two dimer axes were then superimposed using COOT providing pre-aligned starting models for subsequent protein-protein docking and refinement in Rosetta module Docking2³⁰. Biological evidence disfavors binding mode B because that would lead likely to an uncontrolled multimerization between many TLR3-ECDs or to steric clashes with BGN and the membrane of the endosome.

### Validation of the Model

The high scoring docking models in mode A were consistent with posterior prediction of Optimal Docking Areas³⁹. Discrimination of the tightly clustered optimized dimer models in mode A was based on interface analysis choosing the model with the largest interface area and with negative interface formation energy (ΔGᵢ).

The most probable and compelling complex model complies with multiple structural and biological restraints and provides a plausible approximation of the likely binding mode between huTLR3 and huBGN. At present it is not known whether in vivo huBGN binds to hTLR3 in chondroitinated (huBGN⁺) or unchondroitinated form. In vitro core protein interactions of the huBGN C-terminal region suffice to interact with TLR3-ECD, and the model also allows for the possibility that huBGN⁺ binds because there is enough accessible space to accommodate exposed the N-terminally located glycosaminoglycan decorations absent in the crystal structure templates. Similarly, there is sufficient room to accommodate the complex glycans without steric interference between the binding partners. After torsion adjustment of the glycans, the model was refined with the refineInterface module of ICMPro.

Protein interface analysis⁴⁰ indicates a buried surface between the complex dimers of about 800 Å² with −2.4 kcal interface energy. Interface parameters of the obligate hBGN dimer were calculated simultaneously as a cross validation (˜1300 Å² buried surface with −2.0 kcal interface energy). The buried complex interface numbers indicate a weaker interaction than expected for an obligate dimer such as huBGN, but significantly larger than typical for a crystal contact⁴¹. A weak but plausible interaction between huTLR3 and huBGN is consistent with the weak binding interaction observed in the SEC experiments.

The role of the TLR3 loop 336-343 proximal to the binding site remains unresolved. This loop gets cleaved at an unknown position in vivo⁴² and it is unknown whether any residues are lost in the process. While no attempts were made to predict possible additional binding contacts between this hTLR3-ECD loop and hBGN, the model provides enough space to accommodate the loop residues should such an interaction exist.

### Production of Recombinant TLR3 ECD:

The human TLR3-ECD (27-700) was modified with an N-terminal gb67 leader and a C-terminal 6× poly histidine tag and the sequence was produced synthetically (Geneart) and cloned into a pFastBac1 vector. The virus was generated and amplified at large scale levels using a Sf21 insect cell-line (Bac-to-Bac®). One liter Sf21 cell culture was infected and harvested at 72 hours. The culture was centrifuged at 1000×g for 30 min and the supernatant was filtered through a 0.2 μm nitrocellulose membrane. The filtrate was loaded onto a 5 ml His Trap EXCEL column (GE, 17-3712-06) using a peristaltic pump. The column was washed with 10 column volumes (CV) of buffer (20 mM HEPES pH 7.5, 150 mM NaCl) and eluted with 20 mM HEPES pH 7.5, 150 mM NaCl and 500 mM imidazole. The protein fractions were analyzed by SDS-PAGE and TLR3 containing fractions were pooled and concentrated to a final volume of ˜500 μl using a spin concentrator (Vivaspin®20) with a 10 kDa molecular weight cut-off. TLR3 was subjected to size exclusion chromatography (SEC) using Supedex200 increased 10/300 GL column (GE, 28990944) that was previously equilibrated in 20 mM HEPES pH7.5 150 mM NaCl buffer. TLR3 containing fractions were analyzed by SDS-PAGE and pooled and concentrated to 1 mg/ml. Protein concentration was estimated using a Nano Drop by measuring the absorbance at 280 nm (MW=77.4 g mol⁻¹, ε=61.9 mol⁻¹ cm⁻¹).

Production of recombinant hBGN:

The human BGN (38-368) was modified with an N-terminal Secrecon tag followed by a 6× poly histidine tag and a Strep-Tag II. The sequence was produced synthetically (Geneart) and cloned into a pFastBac 1 vector. The virus was generated and amplified at large scale levels using a Sf21 insect cell-line (Bac-to-Bac). One liter Sf21 cell culture was infected ˜ at 1 Mio cells/ml and harvested at 72 hours. The culture was centrifuged at 1000×g for 30 min and the supernatant (SN) subsequently filtered through a 0.2 μm nitrocellulose membrane. The SN was adjusted to 20 mM HEPES pH 7.5, 150 mM NaCl and 5 mM Imidazole and degassed before it was loaded onto a 5 ml His Trap EXCEL column (GE, 17-3712-06) using a peristaltic pump. The column was washed with 10 column volumes (CV) of buffer (20 mM HEPES pH 7.5, 150 mM NaCl, 5 mM imidazole) and eluted with 20 mM HEPES pH 7,5, 150 mM NaCl, 10% glycerol and 500 mM imidazole. The protein fractions were analyzed by SDS-PAGE and BGN containing fractions were pooled and concentrated to a final volume of ˜500 μl using a spin concentrator (Vivaspin®20) with a 10 kDa molecular weight cut-off. Then the protein was dialyzed for 30 min against 20 mM HEPES pH 7.5 and 150 mM NaCl (Pur-A-Lyzer™ Maxi 6000). The purity of the protein was analyzed via SDS-PAGE and the concentration was estimated using a Nano Drop by measuring the absorbance at 280 nm.

### In Vitro Binding Experiments Using Size Exclusion Chromatography:

To obtain core protein of bovine biglycan, purchased biglycan (Sigma, St. Louis, MO) was incubated with Chondroitinase ABC (Merck, Darmstadt, Germany) at 37° C. for 2 h. Digestion of chondroitin sulfate was verified by electrophoresis and subsequent Coomassie-staining. To 185 μl of digested BGN (0.5 mg/ml) 15 μl of TLR3 (1 mg/ml) was added and incubated on ice for 30 min. The sample was subjected to size exclusion chromatography (Supedex200 increased 10/300 GL column) using 20 mM HEPES pH7.5 100 mM NaCl as running buffer. Fractions were collected and analyzed by Immunoblotting as described above.

### In Vitro Binding Experiments Using Co-Immunoprecipitation:

Equal amounts of protein (TLR3 and BGN) were subjected to co-immunoprecipitation with a monoclonal rabbit anti-human BGN antibody directed against human BGN aa 260-368 (Invitrogen, JB71-31). The co-precipitated proteins were washed with CHAPS buffer (30 mM HEPES pH 7.5, 150 mM NaCl, 1% CHAPS), subjected to SDS-PAGE and then blotted onto a nitrocellulose membrane (Amersham™ Protran™ Premium 0.45 μm NC). These membranes were then probed with either an antibody against human TLR3 (Cell Signaling, D10F10) or with an antibody against human BGN (R&D Systems, AF2667). Chemoluminescence was detected using Clarity™ Western ECL Substrate (Bio-Rad).

### Results

### TLR3 is Highly Expressed and Regulated in the Aortic Valve

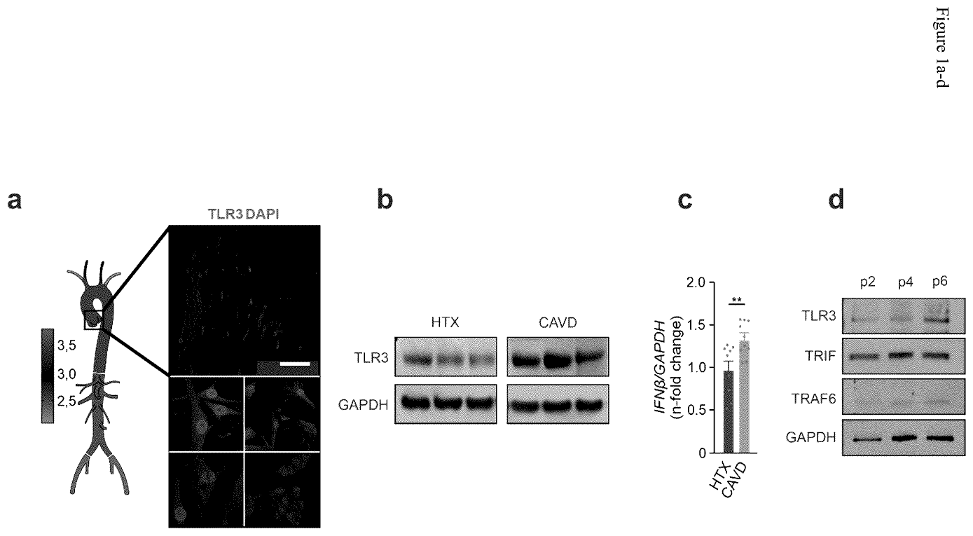

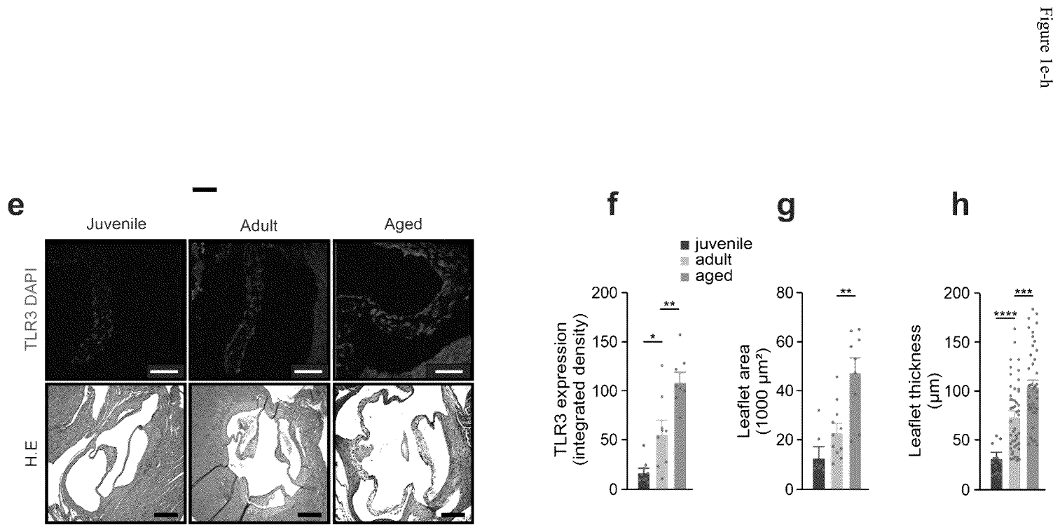

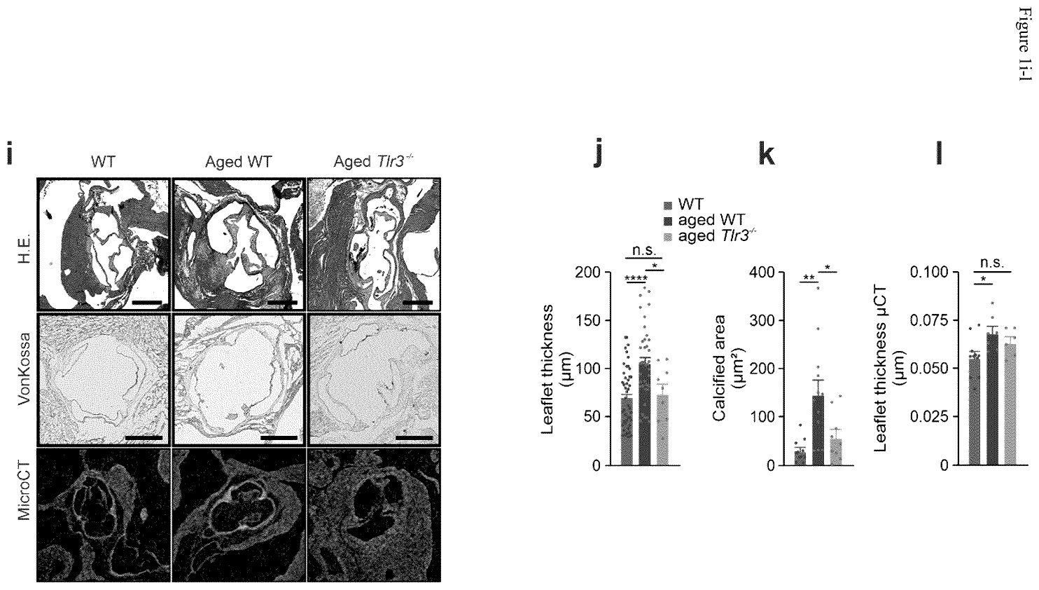

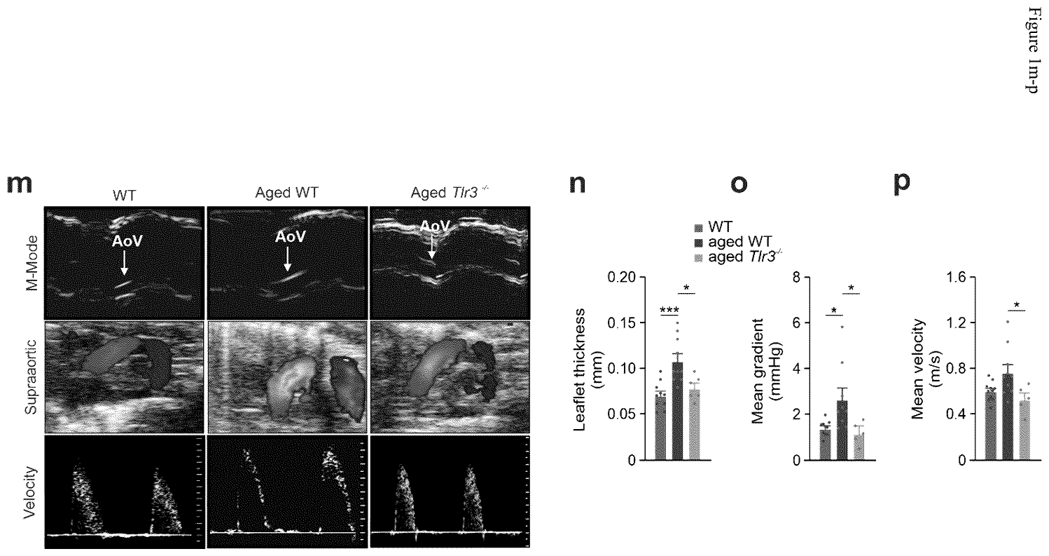

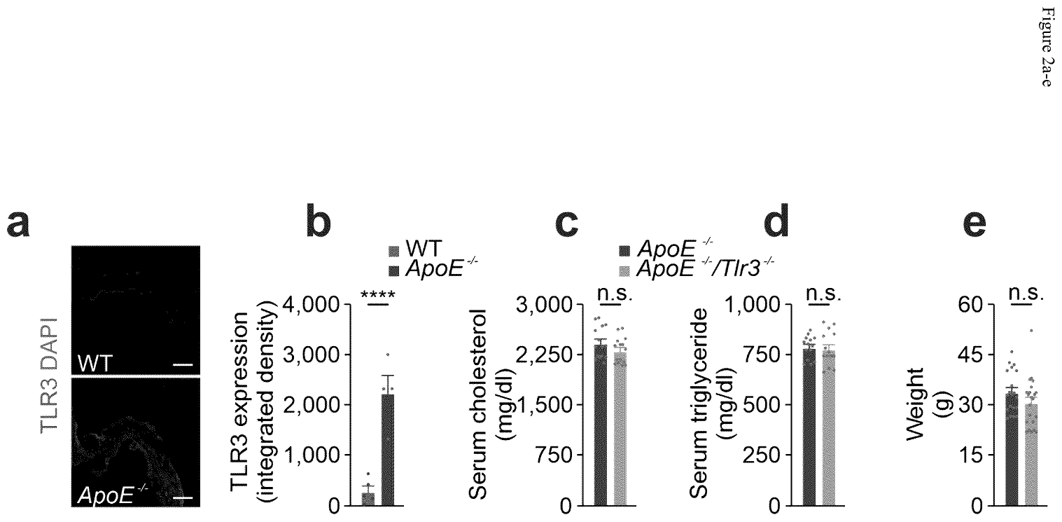

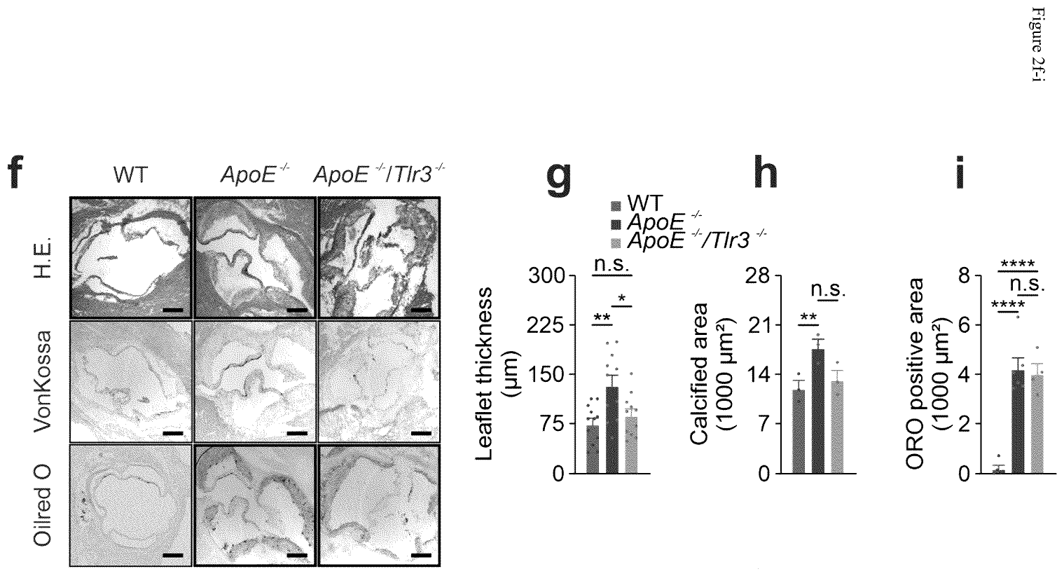

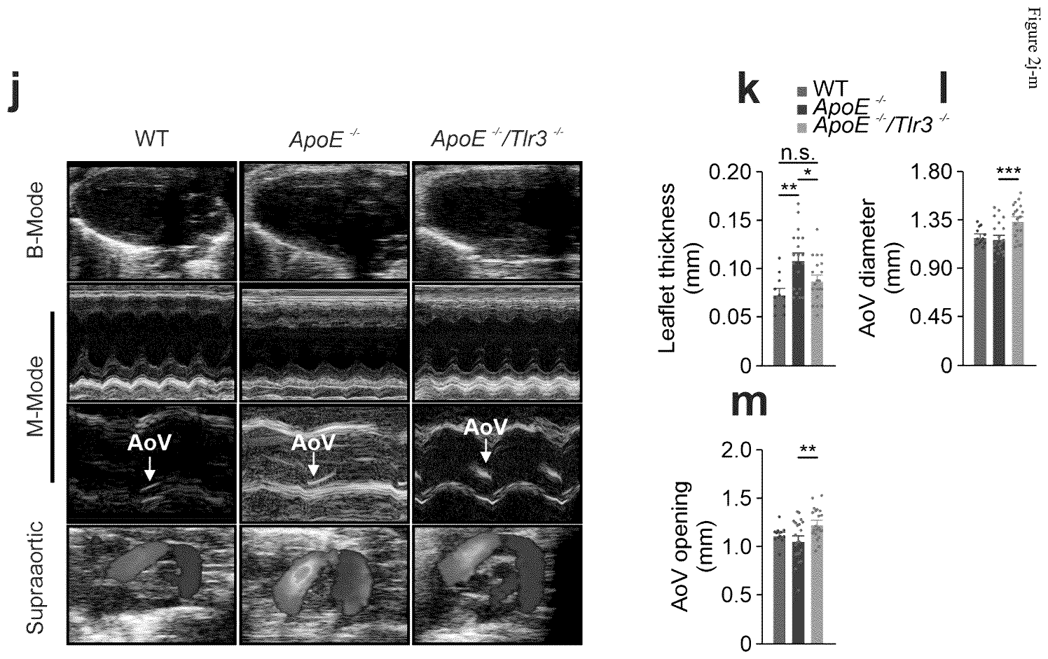

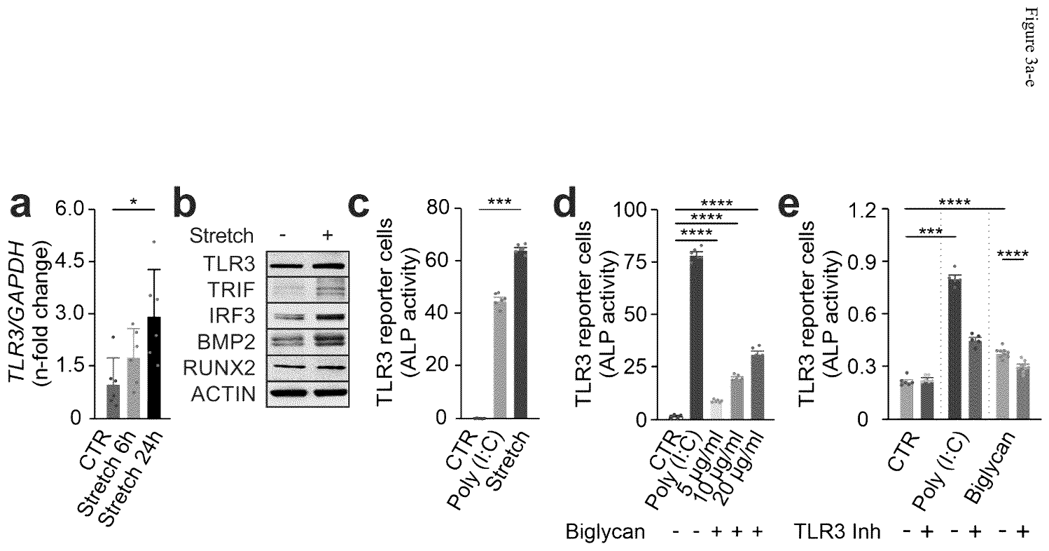

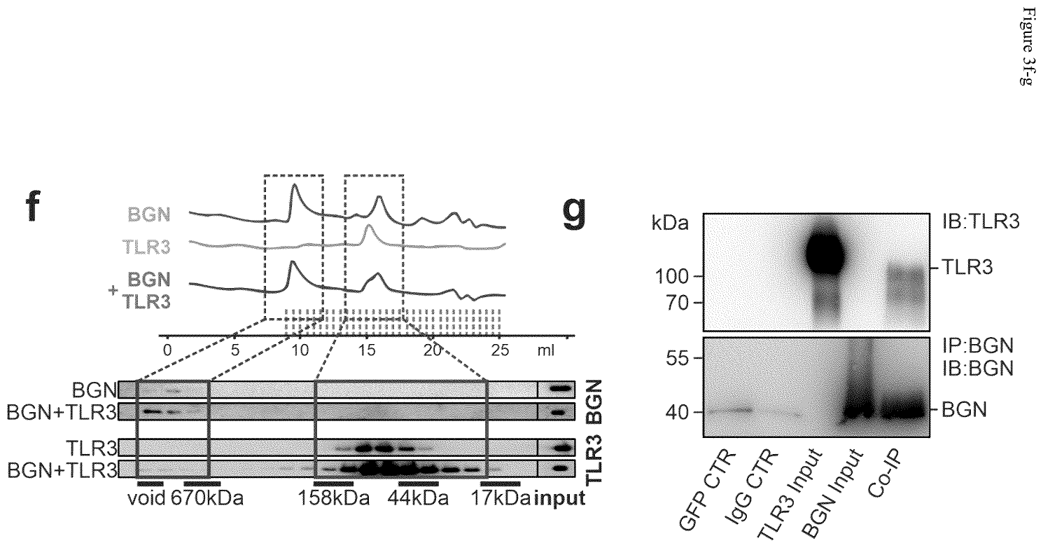

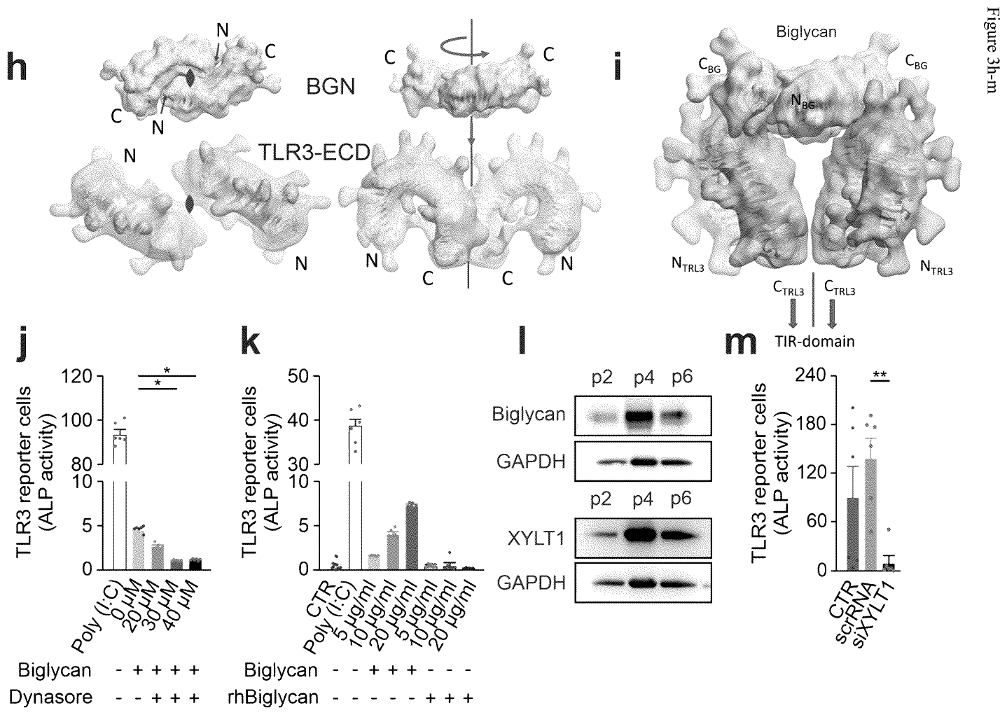

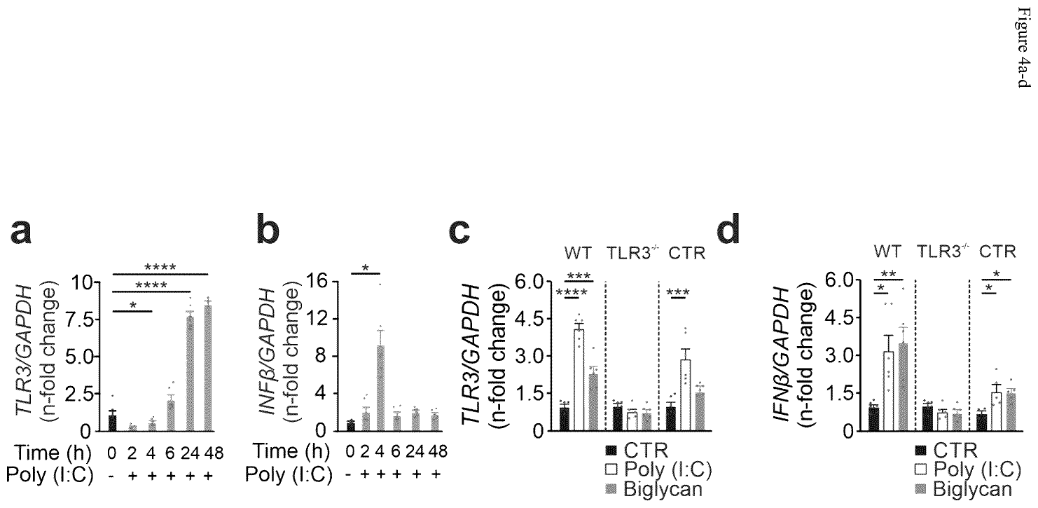

To determine whether TLR3 plays a physiologically relevant role in CAVD pathology, we first examined its expression. In humans, TLRs show vessel-specific expression within the cardiovascular system, depending on their anatomical site⁴³. In mice, Tlr3 was highly expressed in the ascending aorta and aortic valve, with a substantial decrease in the peripheral arteries (FIG. 1A). Similarly, we found abundant expression of TLR3 in human aortic valves (FIG. 1A), which share a common embryonic origin with the ascending aorta⁴⁴. Valvular interstitial cells (VICs) isolated from human aortic valves maintained high TLR3 expression in culture (FIG. 1A). Importantly, both TLR3 and IFN-β showed significantly increased expression in aortic valves from CAVD patients, when compared with healthy controls (FIGS. 1B and 1C), indicating an important role of the TLR3-IFN axis in these patients. With age being the most prominent risk factor for CAVD, with its prevalence increasing significantly in octogenarians,⁴⁵ we analyzed human TLR3 expression in VICs cultured in vitro as a proxy. Indeed, TLR3 protein expression markedly increased at higher passage numbers of cultured VICs (FIG. 1D). In wild-type mice, aortic valve leaflet thickness and area increase with age, indicating initiation of CAVD. In parallel, valvular TLR3 expression increased with age (FIGS. 1E-H). Taken together, TLR3 expression increases with age and with CAVD in the aortic valve of mice and humans.

Tlr3 Deficiency Protects from CAVD

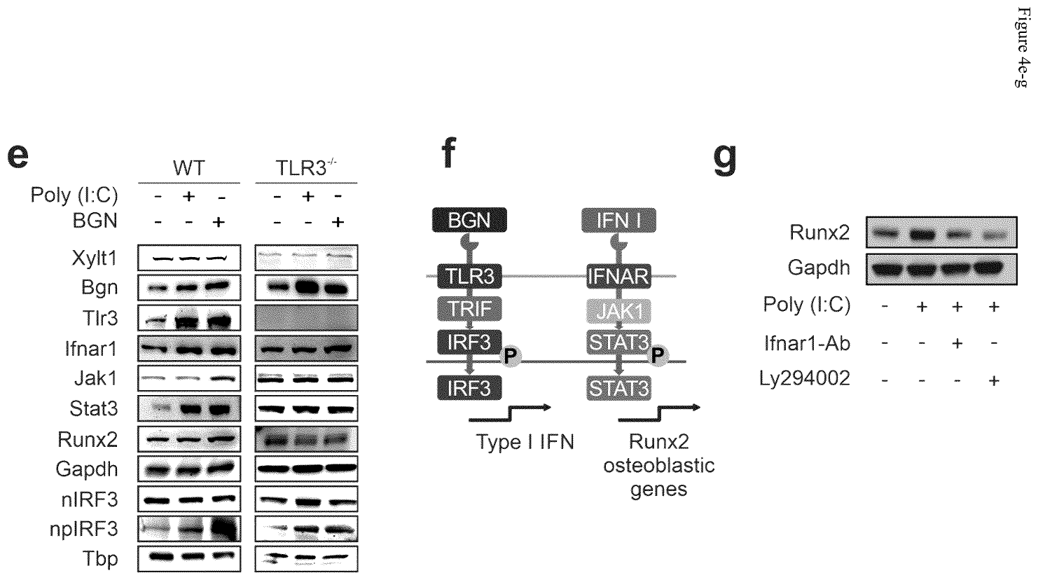

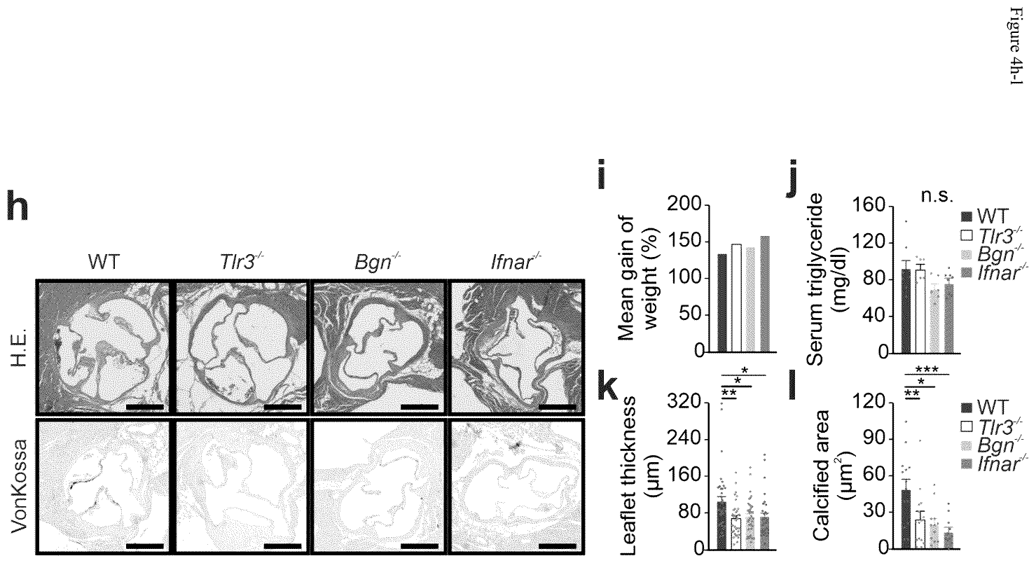

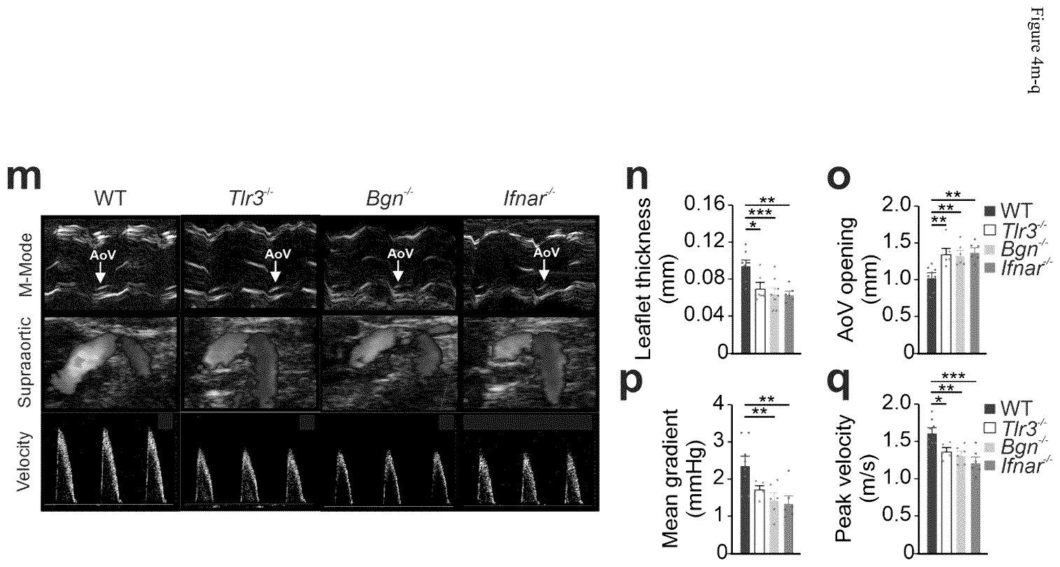

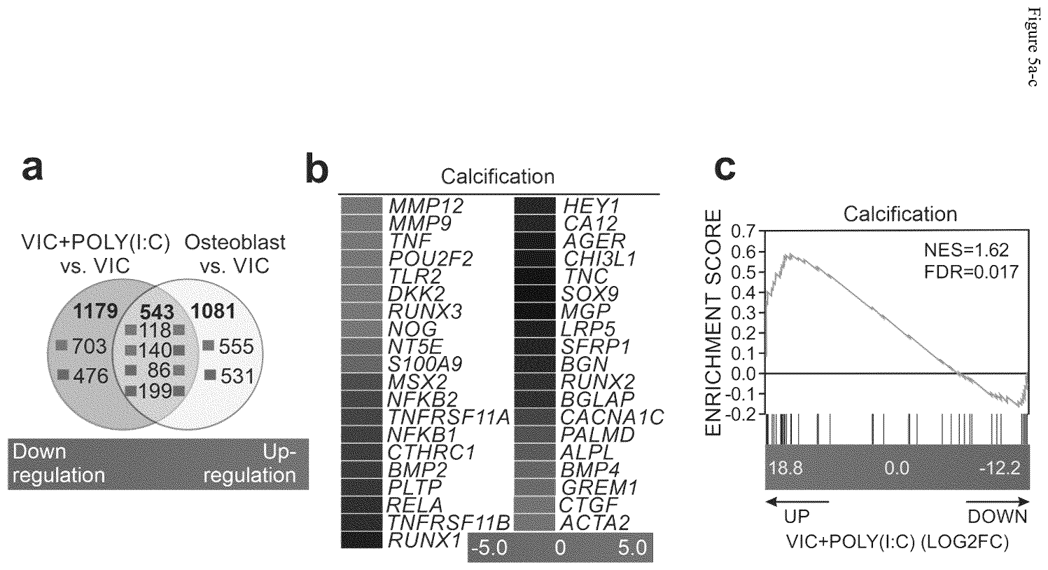

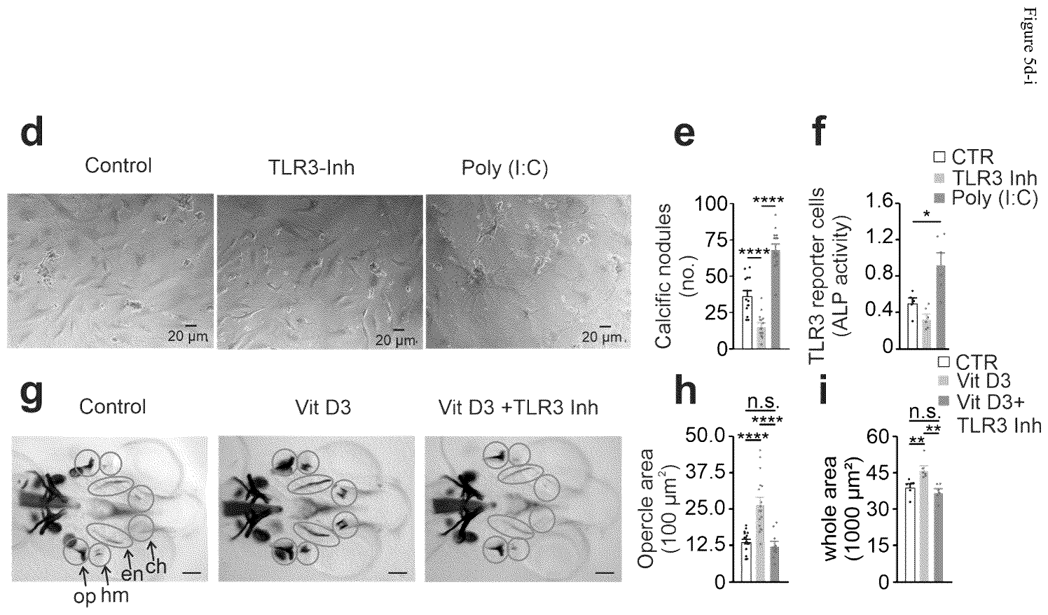

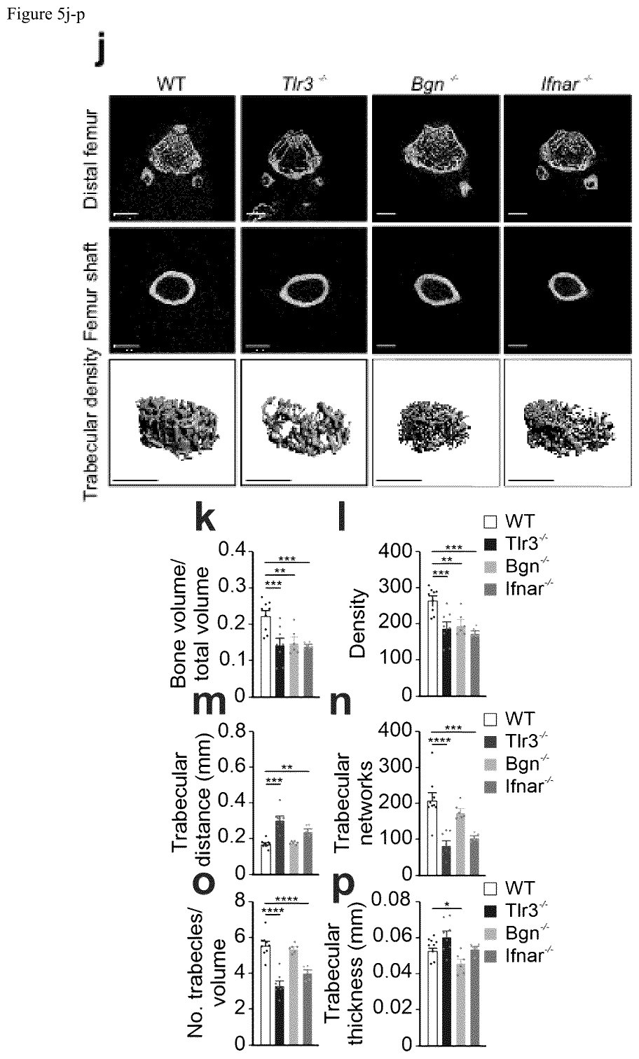

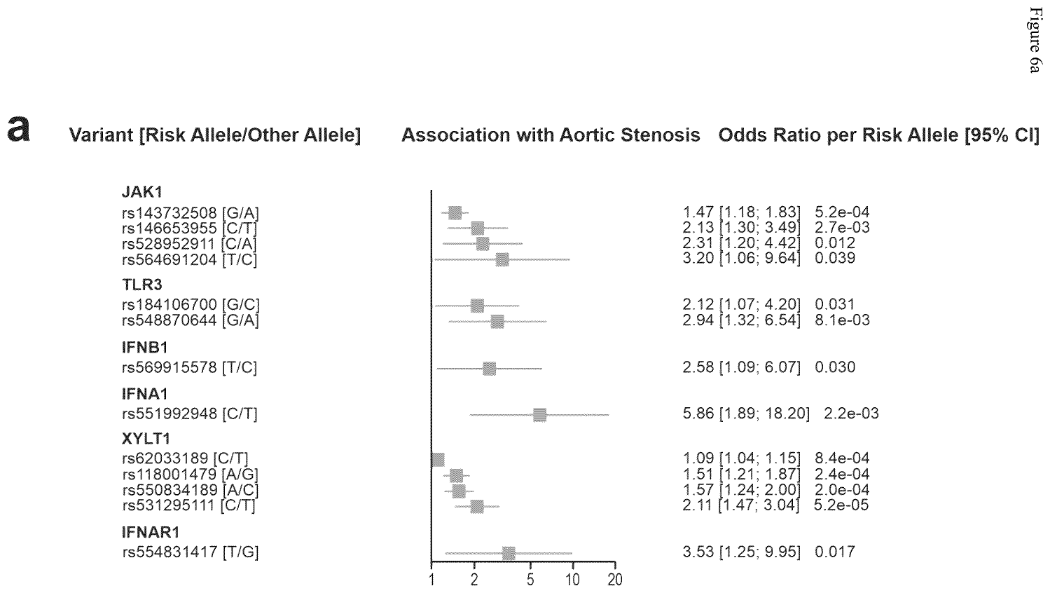

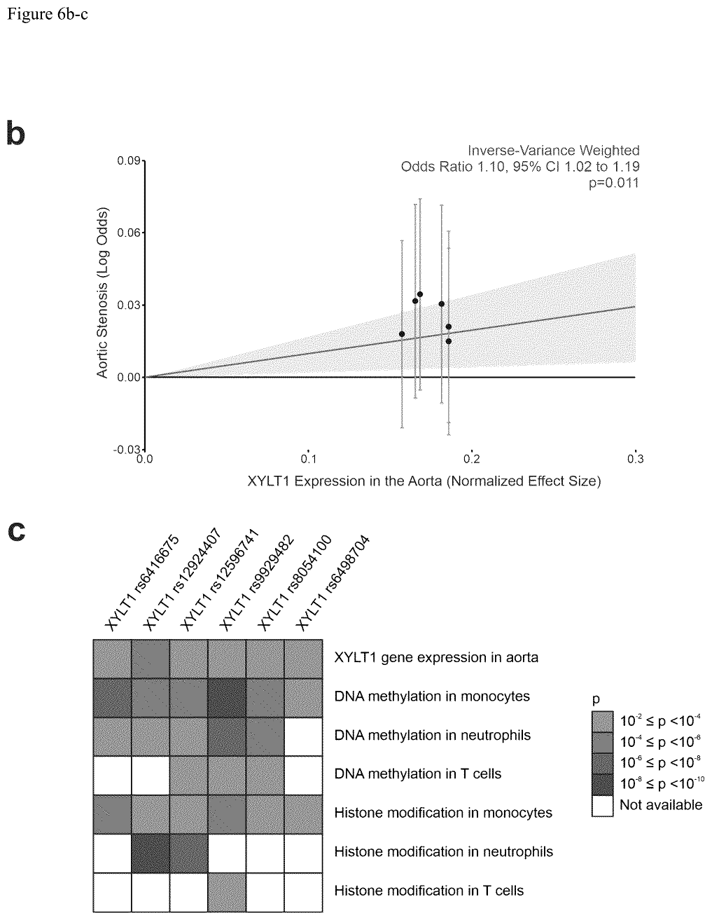

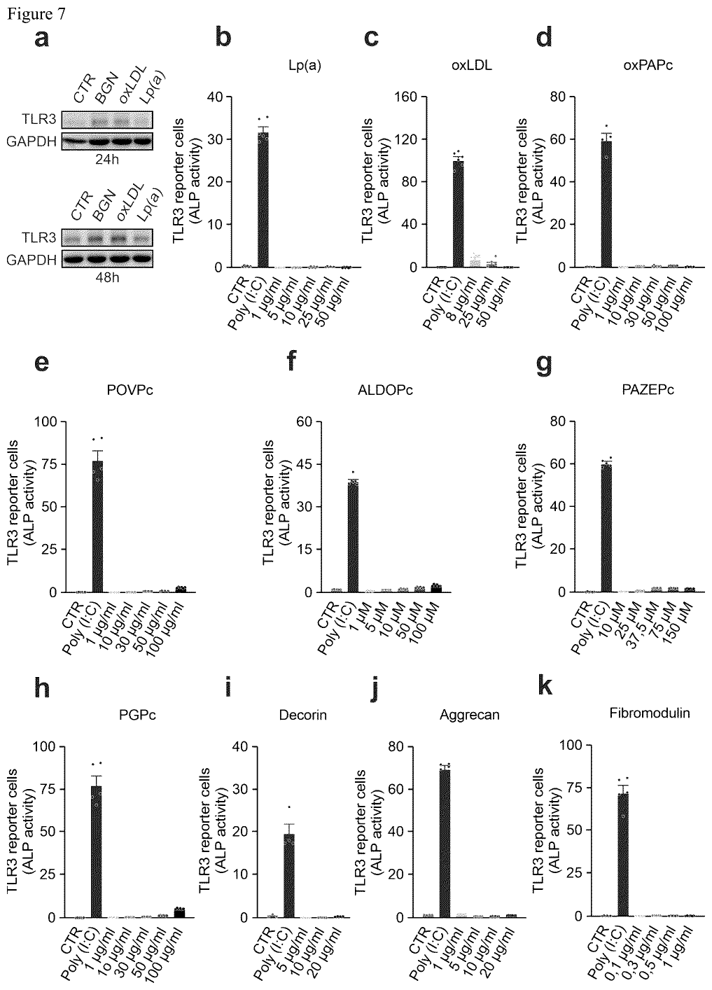

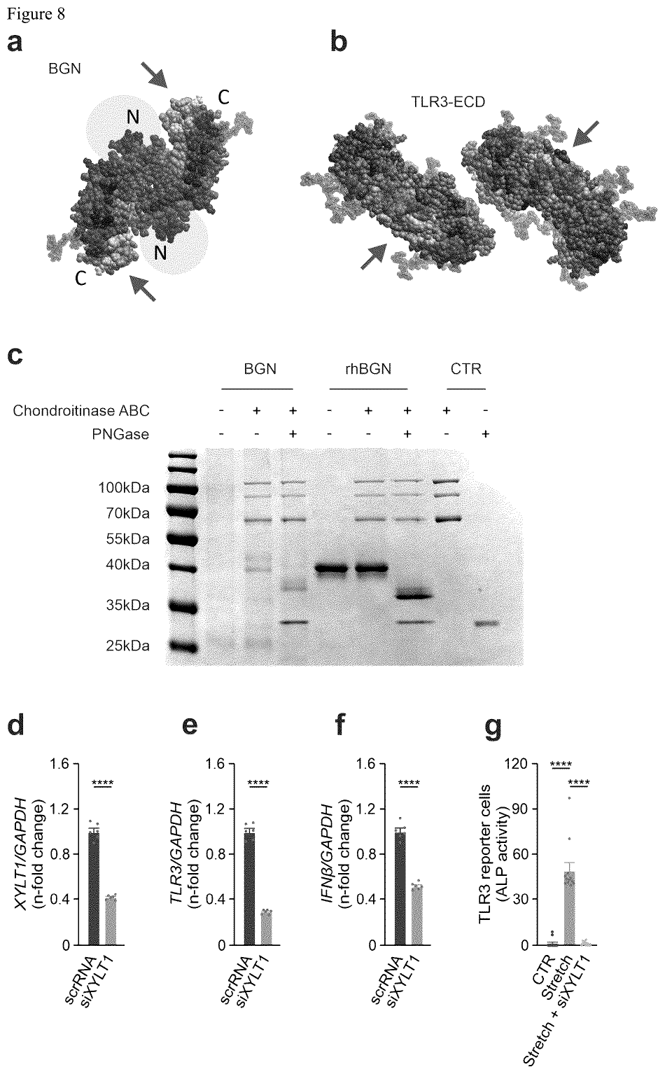

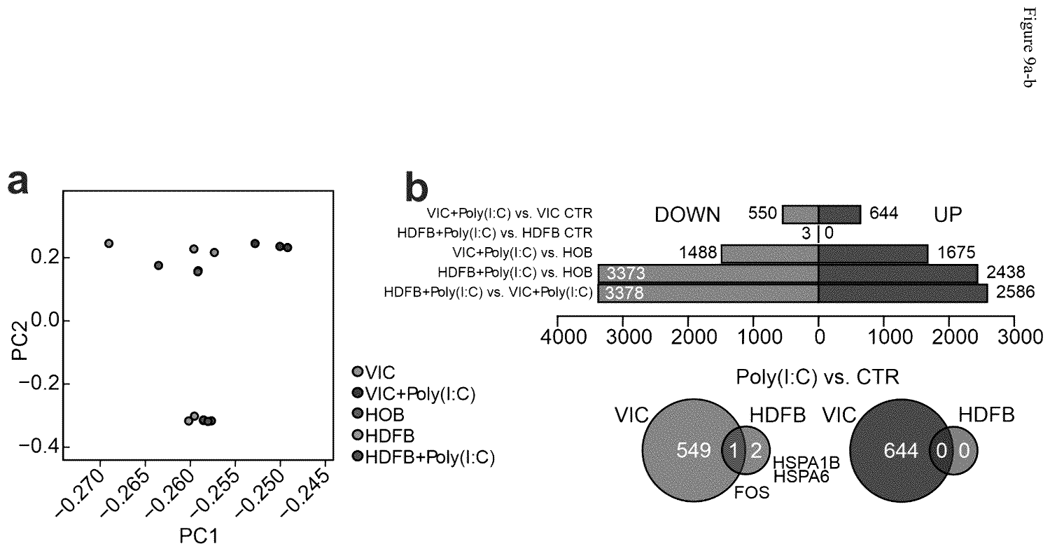

To investigate the physiological importance of Tlr3 in aortic valve pathology, we studied CAVD development in Tlr3^{-/-} mice. Intriguingly, whereas aged wild-type mice exhibited clear signs of aortic valve thickening and calcification, age-matched Tlr3^{-/-} mice were protected from these CAVD phenotypes (FIGS. 1I-L). Furthermore, transthoracic echocardiography revealed increased leaflet thickness, high pressure gradients and increased transvalvular velocities in aged wild-type animals but not aged Tlr3^{-/-} mice (FIGS. 1M-P). We also analyzed ApoE^{-/-} mice which develop CAVD when fed a high fat diet. We found that hyperlipidemia led to the upregulation of TLR3 in the aortic valves of ApoE^{-/-} mice (FIGS. 2A and 2B). Importantly, ApoE^{-/-}; Tlr3^{-/-} double-knockout mice were protected from hyperlipidemia-induced CAVD but did not show differences in serum cholesterol, triglyceride levels or weight (FIG. 2C-M). In summary, our findings provide the first evidence that Tlr3 is critically involved in CAVD development.

### Biglycan is an Endogenous TLR3 Ligand

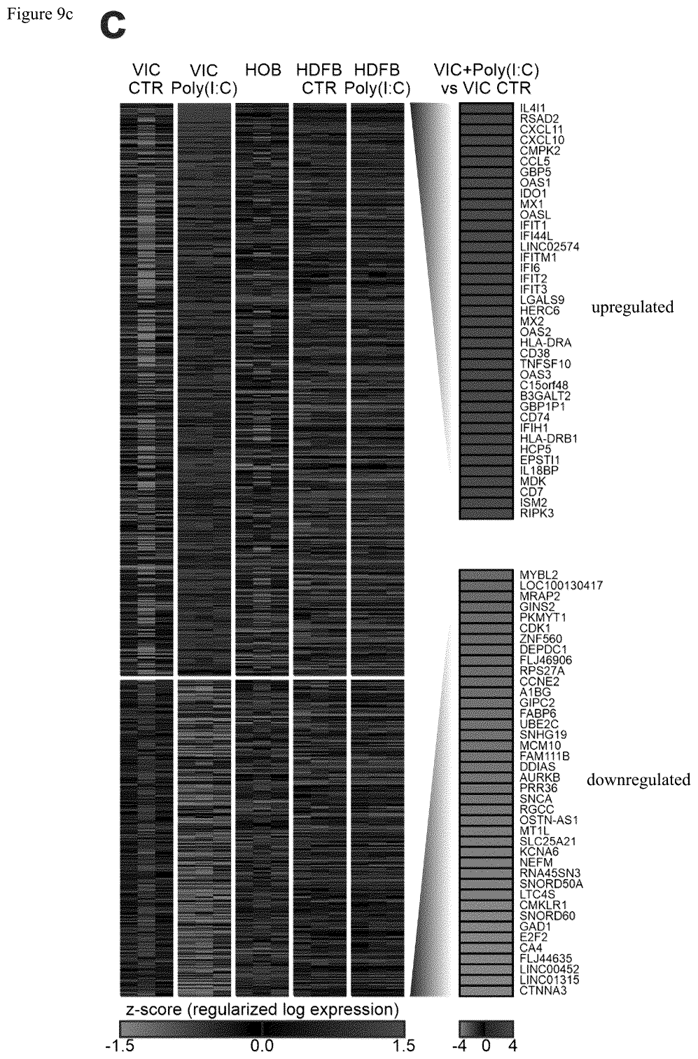

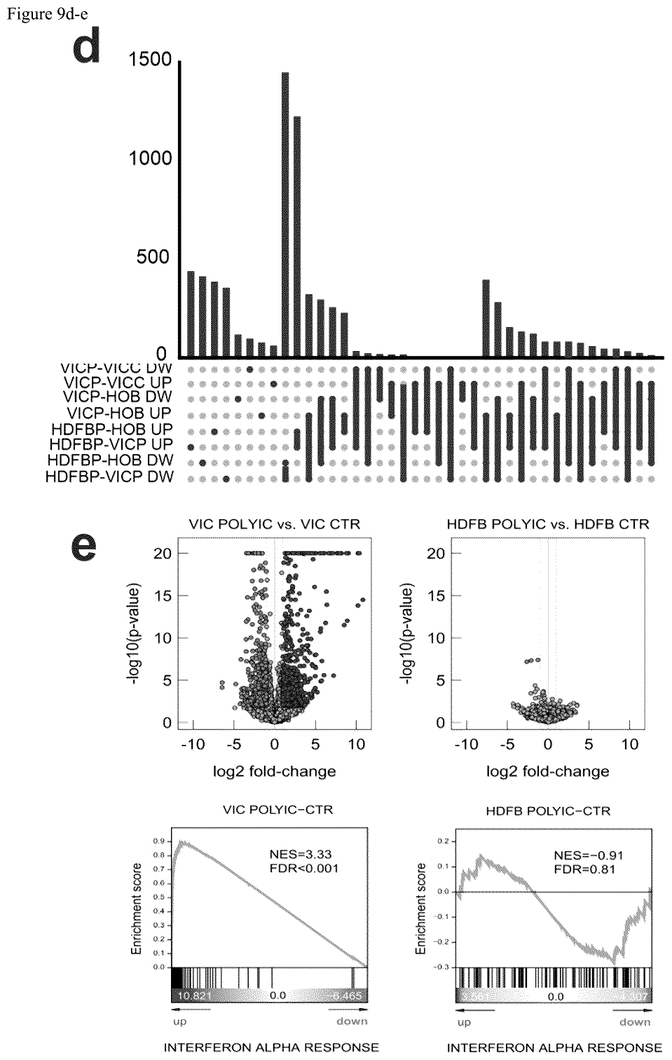

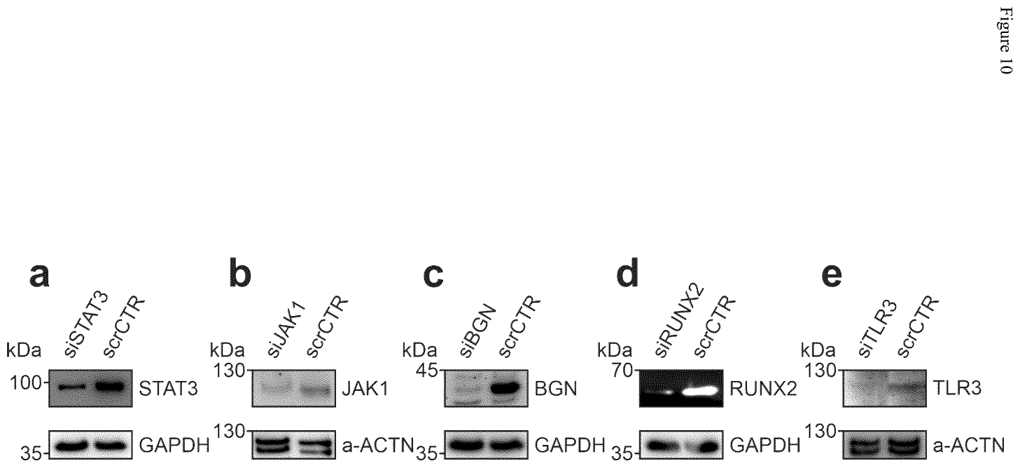

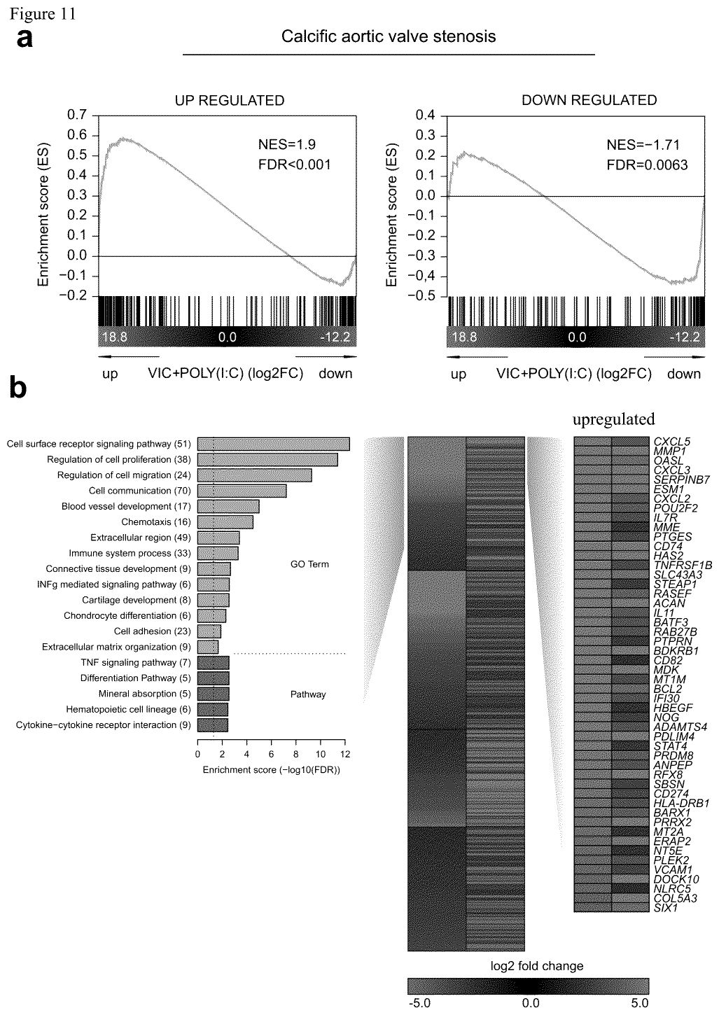

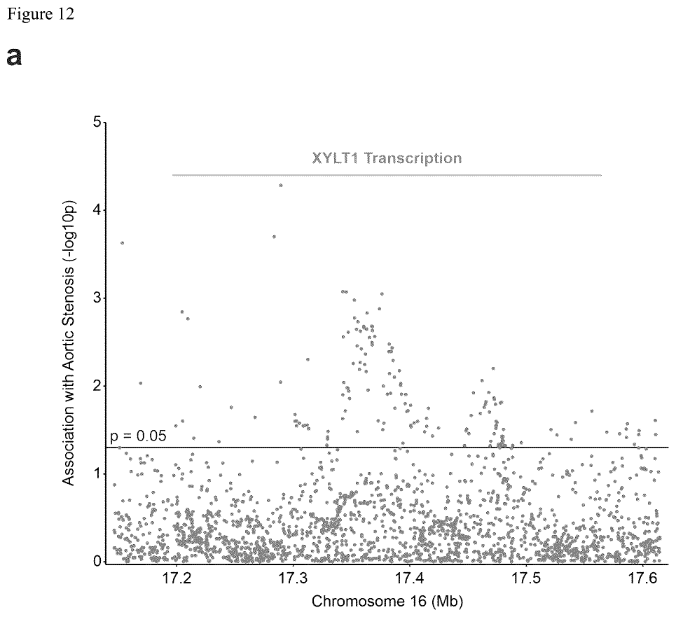

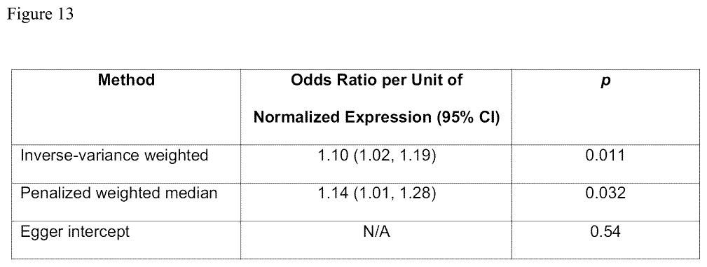

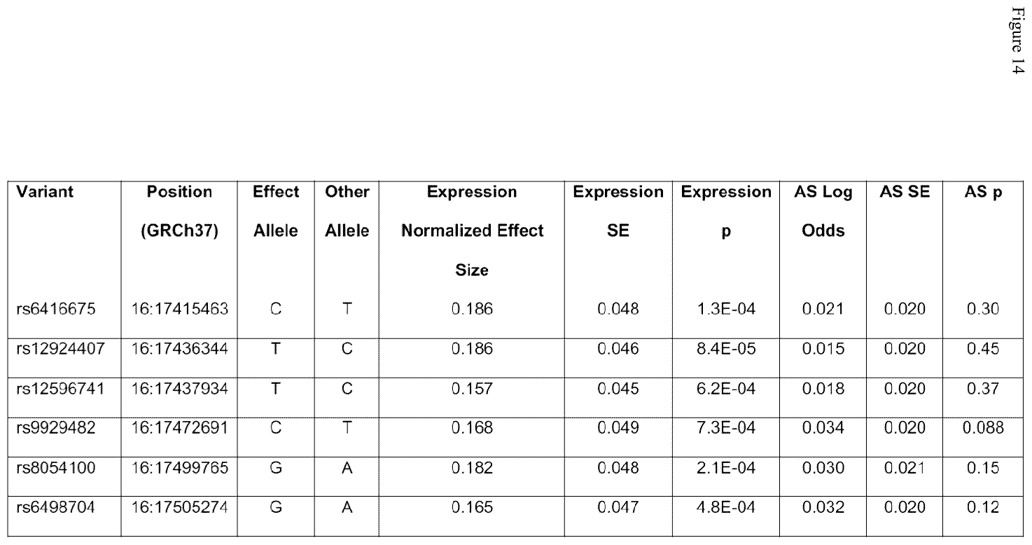

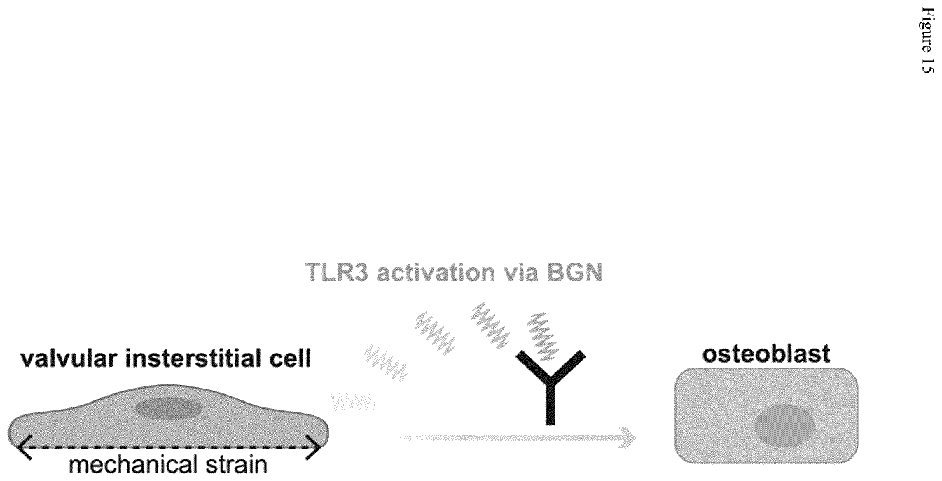

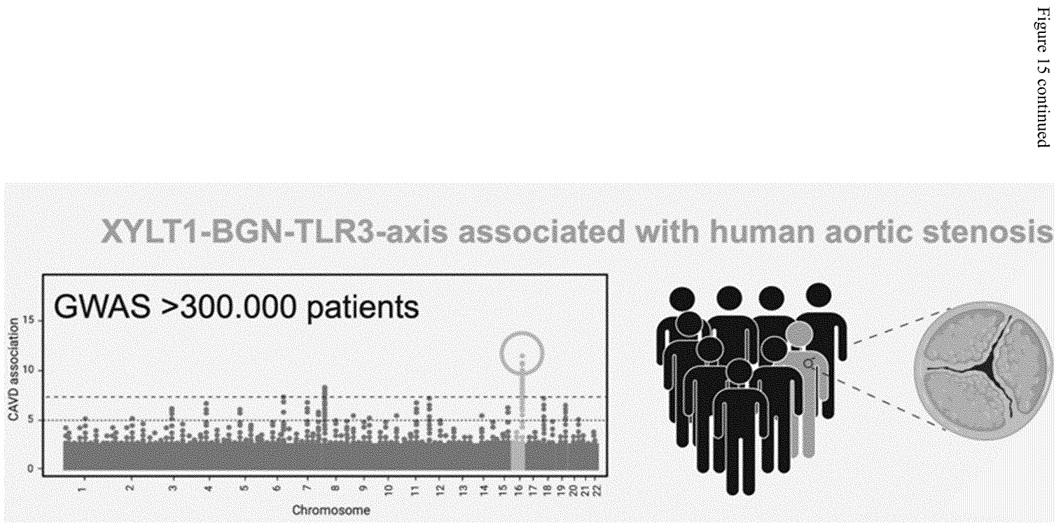

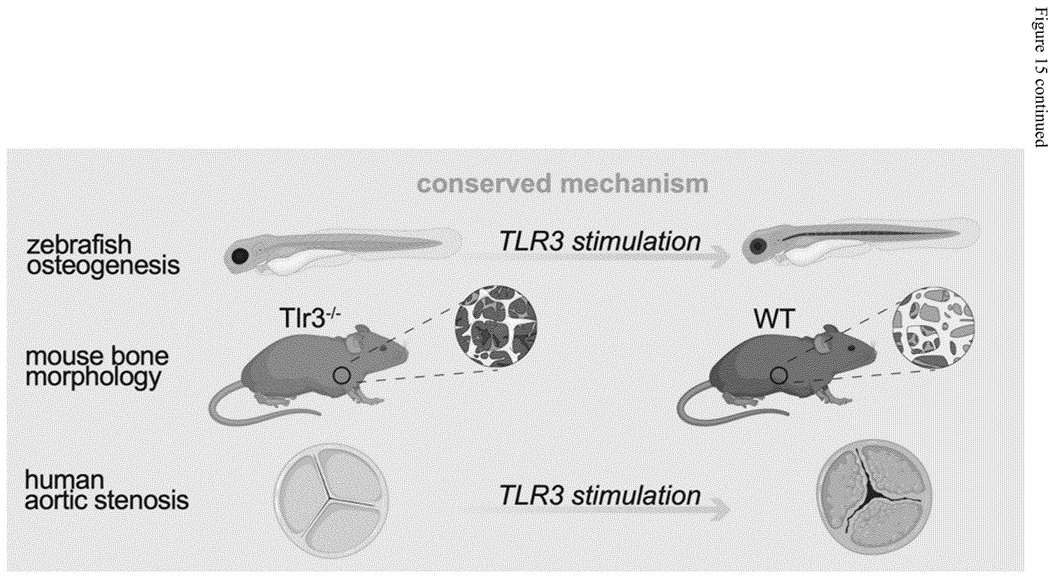

We next tested the hypothesis that high-pressure gradients in CAVD and associated mechanical strain may lead to the release of an endogenous TLR3 ligand from the ECM within VICs. To test this hypothesis, we subjected cultured VICs to mechanical strain, and indeed we observed increased expression of TLR3 as well as TRIF and IRF3 (FIGS. 3A and 3B). Moreover, we found that VICs subjected to mechanical strain showed increased expression of both BMP2 and RUNX2 (FIG. 3B). Of note, supernatants from mechanically stretched VICs activated TLR3 reporter cells, suggesting the release of an endogenous TLR3-activating ligand (FIG. 3C). We next aimed to systemically investigate potential endogenous TLR3 ligands involved in CAVD and VIC calcification. We first considered modified bioactive lipids contained in oxidized low-density lipoproteins (oxLDL) as well as lipoprotein (a) (Lp(a)), which are associated with CAVD in humans 46,47. Although oxLDL induced the expression of TLR3 in VICs (FIG. 7A), neither oxLDL or oxLDL-associated phospholipids, nor Lp(a) activated TLR3 in reporter cells (FIGS. 7B and 7C). We screened other putative ligands and found that the proteoglycan biglycan (BGN), a structural protein of the extracellular matrix (ECM), not only induced TLR3 expression in VICs but also activated TLR3 reporter cells in a dose-dependent fashion (FIG. 3D, FIGS. 7D-K). These effects were strictly dependent on TLR3, as they were abolished upon TLR3 inhibition (FIG. 3E). To demonstrate a physical interaction between BGN and TLR3, we incubated purified human TLR3 ectodomain (huTLR3-ECD) with BGN and performed size-exclusion chromatography followed by immunoblotting, which revealed co-elution (FIG. 3F). Immunoprecipitation of BGN and huTLR3-ECD confirmed this interaction (FIG. 3G).

The huTLR3-ECD forms a horseshoe structure that is connected to the transmembrane helix via its C-terminal domain. Binding of a ligand leads to huTLR3 homodimer formation and initiates the TLR3 signaling cascade 48,49. The BGN core protein forms obligate dimers 37, suggesting that TLR3 dimerization may be induced upon binding a BGN dimer (FIG. 3H).

To illustrate the most plausible interaction between huTLR3 and huBGN, we modelled the complex maintaining the collinearity of the dimer axes as a restraint in subsequent protein-protein docking and local refinement^{30,38} (FIG. 3I). Binding site analysis and the absence of steric interference with glycan decorations support the proposed interaction model (FIGS. 8A and 8B). These data further support a direct interaction between BGN and TLR3. TLR3 is expressed mainly on endosomes⁵⁰. Accordingly, we observed that BGN-mediated activation of TLR3 reporter cells was abolished upon inhibition of endocytosis (FIG. 3J).

### Maturation of BGN Via XYLT1 is Crucial for TLR3 Activation

Biological activity of BGN is primarily dependent on its chondroitin sulfate chains⁵¹. Accordingly, BGN lacking chondroitin sulfate chains (rhBGN) (FIG. 8C) did not activate TLR3 in reporter cells (FIG. 3K). Xylosyltransferases (XYLT) add chondroitin sulfate chains to BGN, representing an important step in BGN maturation. Both BGN and XYLT1 protein expression were upregulated in aged VICs (FIG. 3L). Moreover, supernatant obtained from XYLT1-deficient VICs did not activate TLR3 reporter cells (FIG. 3M) nor IFN-β transcription (FIGS. 8D-F), irrespective of mechanical stretching, in contrast to supernatant obtained from wild-type VICs (FIG. 8G). Altogether, our data suggest that XYLT1-mediated addition of chondroitin sulfate chains for maturation of BGN is critically required for TLR3 activation. Intriguingly, mutations in XYLT1 are associated with impaired calcification 52, corroborating the implication of the XYLT1-BGN axis in calcification.

### The BGN-TLR3-IFNAR1 Axis Induces Calcification

Binding of IFN-β to its receptor IFNAR was previously shown to promote expression of Bone morphogenic protein 2 (BMP2) and the pivotal osteoblastic transcription factor RUNX2 in murine osteoblasts⁵³. To determine if activation of the TLR3-IFN axis is sufficient to trigger this pathway, we treated VICs with the well-established synthetic TLR3 agonist Poly(I:C). We found that VICs treated with Poly(I:C) had increased expression of IFN-β and TLR3 (FIGS. 4A and B). To test whether the observed effects were TLR3 specific, we repeated the experiments in human dermal fibroblasts lacking TLR3. Although expression profiles of human dermal fibroblasts and VICs are not identical upon TLR3 stimulation (FIGS. 9A-E), both BGN and poly(I:C) induced transcription of TLR3 and IFN-β in wild type and empty vector control cells, whereas there was no response upon stimulation in Tlr3^{-/-} cells (FIGS. 4C and D). Both poly(I:C) and BGN treatment led to phosphorylation of IRF3, upregulation of IFNAR1 and associated phosphorylation of JAK1/STAT3, resulting in increased levels of the essential osteoblastic transcription factor RUNX2 in wild type and empty vector control cells. Again, there was no activation of the TLR3-IFN axis nor upregulation of Runx2 in Tlr3^{-/-} cells upon treatment (FIG. 4E). Specificity of antibodies was tested beforehand (FIG. 10A-E).

To corroborate the newly identified TLR3-RUNX2 pathway (FIG. 4F), we abolished IFN-β synthesis with the PI3K inhibitor LY294002⁵⁴ or blocked IFNAR1 activity with a specific antibody. Pre-treatment of VICs with LY294002 or the specific IFNAR1 blocking antibody prevented RUNX2 upregulation upon Poly(I:C) treatment (FIG. 4G).

### Role of the BGN-TLR3-IFNAR1 Axis in CAVD In Vivo

To finally proof the physiological role of the identified BGN-TLR3-IFNAR1 axis in vivo, the hypercholesterinemia-induced model of CAVD was induced in Tlr3^{-/-}, Bgn^{-/-} and Ifnar1^{-/-} mice. Animals were fed a high fat diet for 4 months and analyzed thereafter for their valvular phenotype²⁰ (FIG. 4H). Mean gain of weight and serum triglyceride levels were consistent between the groups (FIGS. 41 and 4J). Aortic valve leaflet thickness was increased in wild-type mice, whereas deficiency of Tlr3, Bgn or Ifnar1 protected from valvular thickening (FIG. 4K). Hypercholesterinemia induced aortic valve calcification in wild-type but not in Tlr3^{-/-}, Bgn^{-/-} and Ifnar1^{-/-} mice (FIG. 4L). Functional analysis of the aortic valves via transthoracic echocardiography confirmed that deficiency of Tlr3, Bgn or Ifnar1 protected from valvular thickening and hemodynamic consequences of CAVD, as animals showed no changes in aortic valve opening, mean transvalvular gradient or peak velocity in contrast to wild-type animals (FIG. 4M-Q). These results strongly support the significance of the BGN-TLR3-IFNAR1 axis in the development of CAVD.

### TLR3 Activation of VICs Induces an Osteoblast-Like Geno- and Phenotype

Next, we performed RNA-seq analyses to comprehensively investigate whether the mechanism of TLR3-RUNX2 pathway activated in Poly(I:C)-treated VICs also occurs in osteoblasts. We found that 118 genes were upregulated and 199 genes were downregulated in both Poly(I:C)-treated VICs and in human osteoblasts compared to untreated VICs (FIGS. 11A and 11B). Gene set enrichment analysis of the Poly(I:C)-treated VICs revealed that numerous of the upregulated genes are involved in calcification and CAVD (FIGS. 5C, 11A and 11B). Thus, Poly(I:C) treatment of VICs induces an osteoblastic transcriptome. Finally, Poly(I:C) treatment of VICs enhanced the production of calcific nodules and increased the activity of the osteoblastic enzyme alkaline phosphatase (ALP)⁵⁵, whereas TLR3 inhibition using a dsRNA/TLR3 complex inhibitor reduced calcification and ALP activity (FIGS. 5D-F). Overall, these data heavily suggest that TLR3 stimulation in VICs activates an osteoblast-related pathway to promote calcification. This finding goes in line with the established concept of osteoblasts being sophisticated fibroblasts⁵⁶.

### TLR3 Activation Promotes Calcification and Bone Development In Vivo

We finally tested the hypothesis that TLR3 may be involved in the maturation of fibroblasts into osteoblasts beyond the aortic valve. TLRs are highly conserved among species and drive dorso-ventral polarity in Drosophila melanogaster⁵⁷. The TLR3 analog tollo is of central importance for axial development in Drosophila melanogaster by activating the transcription factor runt, the analog of vertebrate RUNX2⁵⁷. To test whether TLR3 plays an evolutionary conserved role in morphogenesis, we targeted Tlr3 in zebrafish (Danio rerio). Zebrafish are a well-established model organism to study calcification and bone formation. The opercle and the branchiostegal rays develop in the zebrafish embryo at 8-10 days post fertilization (dpf), and premature calcification of these hyomandibular bones can be induced by supplementation with vitamin D3 (VitD3) 26. VitD3-treated larvae displayed early and strong calcification of landmark bones, which was abolished by co-incubation with a TLR3 inhibitor (FIGS. 5G-I). These findings suggest that TLR3 activation promotes cellular calcification during bone development in zebrafish. It is known that Ifnb^{-/-} and Stat3^{-/-} mice exhibit a distinctive bone phenotype with impaired bone formation and osteoporosis 58,59. To analyze whether the BGN-TLR3-IFNAR1 axis has an impact on bone structure, we performed micro-computed tomography of femurs from Tlr3^{-/-}, Bgn^{-/-} and Ifnar1^{-/-} mice. All knock-out animals revealed a distinct osteoporotic phenotype with decreased bone volume and bone density compared to WT animals (FIGS. 5J-L). Moreover, trabecular architecture was altered upon in the knockouts (FIGS. 5M-P). Together, these findings demonstrate that Tlr3 plays a role in bone development and calcification in both zebrafish and mice.

Genetic Association of TLR3 Signaling with CAVD

To determine whether genetic variation at genes implicated in this TLR3 signaling pathway are associated with diagnosed aortic stenosis in humans, we examined two large-scale cohorts (Genetic Epidemiology on Adult Health and Aging (GERA), n=55,192 with 3,469 aortic stenosis cases; UK Biobank, n=257,231 with 2,213 aortic stenosis cases). We discovered 307 variants that were nominally significant (p≤0.05) for aortic stenosis in a meta-analysis of the UK Biobank and GERA. Notably, 16 variants in the JAK1, TLR3, IFNB1, IFNA1, XYLT1, and IFNAR1 loci, representing 13 independent signals, demonstrated strong associations (p≤1×10⁻³) and/or two-fold or greater (up to 5.86-fold) odds of aortic stenosis (FIG. 6A and FIG. 12). These variants were rare (minor allele frequency <0.01), except for three variants at the XYLT1 locus. Variants throughout the XYLT1 locus demonstrated significant associations with aortic stenosis in the meta-analysis (FIG. 6 and FIG. 12) Mendelian randomization indicated that genetically-elevated XYLT1 expression in the aorta was associated with greater odds of aortic stenosis, providing evidence for a causal effect (odds ratio per unit of normalized expression, 1.10; 95% CI 1.02 to 1.19; p=0.011) (FIG. 6B and FIG. 13). All six variants in the genetic instrument for aorta expression were also associated with DNA methylation and histone modification in several inflammatory cell types (FIG. 6C). Thus, genetic variation at loci relevant to the TLR3 pathway are associated with clinically relevant aortic valve calcification. In particular, genetic evidence supports XYLT1 as a potential therapeutic target for aortic stenosis.

### Discussion

Our work identifies TLR3 as pivotal part of a conserved mechanism to promote calcification during both bone development and aortic valve calcification. We unveil that a central component of the ECM, namely BGN, induces not only the expression of TLR3 but also a dramatic phenotypic switch of VICs into bone-forming cells. BGN is a proteoglycan consisting of a core protein with two chondroitin sulfate side chains. It stabilizes the ECM and is released upon cellular stress⁶⁰, in turn recruiting macrophages and dendritic cells via TLR2/TLR4, which may contribute to inflammaging and CAVD⁶¹. We provide the first evidence that BGN directly interacts with TLR3 in the cell type primarily responsible for aortic valve calcification. Our results go in line with recent reports linking expression of TLR3 in human fibroblasts to the control of basal IFN-β levels and thus, restriction of viral infection⁶².

Our results indicate that only a mature form of BGN acts to activate TLR3, with chondroitin sulfate side chains constituting a key factor for the biological activity of this proteoglycan. Enzymes responsible for the modification of BGN side chains, including XYLT1, may thus critically determine the fate of BGN and the initiation and progression of CAVD. From an evolutionary perspective, aiming at understanding the implication of the newly identified XYLT1-BGN-TLR3-IFNAR1 pathway in the context of calcification per se, it is important to note that loss of XYLT1 results in impaired chondrocyte maturation and skeletal defects⁵². In line, we found that Tlr3^{-/-} mice as well as zebrafish treated with a Tlr3 inhibitor display a severely impaired calcification of the skeleton. Thus, we describe for the first time a unique pathway of calcification at the interface of innate immunity and bone formation. Moreover, our results suggest that type I IFN might play a pivotal role in physiological and pathological calcifications of conjunctive tissue in general, including musculoskeletal tissue and the cardiovascular system. This finding goes in line with recent reports linking interferonopathies with increased basal interferon levels to pathological calcifications in the brain and the heart⁶³.

We propose that pharmacological inhibition of TLR3 might represent a promising approach to treat CAVD. Of note, TLR3 inhibition was successfully achieved in experimental settings via small molecule inhibitors or blocking antibodies^{1,2}. However, whether targeting TLR3 signaling might come at risk of viral infection with Herpes simplex enzephalitis, influenza or COVID has yet to be determined⁶⁴⁻⁶⁶.

In conclusion, we unravel a novel mechanism driving CAVD, in which mechanically released BGN (i) increases protein expression of TLR3 in valvular interstitial cells, and (ii) mature BGN modified by XYLT1 constitutes a selective and potent endogenous TLR3 ligand, perpetuating valvular calcification via type I IFN signaling. Our data unravel the XYLT1-BGN-TLR3-IFNAR1 axis as an evolutionary conserved pathway of morphogenesis and calcification, offering novel therapeutic strategies to detect and counteract CAVD in humans.

### FIRST LIST OF REFERENCES

Abagyan, R., Totrov, M., and Kuznetsov, D. (1994). ICM: a new method for protein modeling and design. Applications to docking and structure prediction from the distorted native conformation. J Comp Chem 15, 488-506.
Aceto, J., Nourizadeh-Lillabadi, R., Maree, R., Dardenne, N., Jeanray, N., Wehenkel, L., Alestrom, P., van Loon, J. J., and Muller, M. (2015). Zebrafish Bone and General Physiology Are Differently Affected by Hormones or Changes in Gravity. PLOS One 10, e0126928.
Anderson, K. V., Bokla, L., and Nusslein-Volhard, C. (1985). Establishment of dorsal-ventral polarity in the Drosophila embryo: the induction of polarity by the Toll gene product. Cell 42, 791-798.
Babelova A, Moreth K, Tsalastra-Greul W, Zeng-Brouwers J, Eickelberg O, Young M F, et al. (2009). Biglycan, a danger signal that activates the NLRP3 inflammasome via toll-like and P2X receptors. J Biol Chem, 284(36), 24035-48.
Back, M., Gasser, T. C., Michel, J. B., and Caligiuri, G. (2013). Biomechanical factors in the biology of aortic wall and aortic valve diseases. Cardiovasc Res 99, 232-241.
Balachandran K, Bakay M A, Connolly J M, Zhang X, Yoganathan A P, Levy R J (2011). Aortic valve cyclic stretch causes increased remodeling activity and enhanced serotonin receptor responsiveness. Ann Thorac Surg, 92(1), 147-53.
Ballinger M L, Nigro J, Frontanilla K V, Dart A M, Little P J (2004). Regulation of glycosaminoglycan structure and atherogenesis. Cell Mol Life Sci, 61(11), 1296-306.
Baumgartner, H., Falk, V., Bax, J. J., De Bonis, M., Hamm, C., Holm, P. J., Iung, B., Lancellotti, P., Lansac, E., Munoz, D. R., et al. (2017). 2017 ESC/EACTS Guidelines for the management of valvular heart disease: The Task Force for the Management of Valvular Heart Disease of the European Society of Cardiology (ESC) and the European Association for Cardio-Thoracic Surgery (EACTS). Eur Heart J.
Bell, J. K., Botos, I., Hall, P.R., Askins, J., Shiloach, J., Segal, D. M., and Davies, D. R. (2005). The molecular structure of the Toll-like receptor 3 ligand-binding domain. Proc Natl Acad Sci USA 102, 10976-10980.
Bittner, V.A., Szarek, M., Aylward, P.E., Bhatt, D. L., Diaz, R., Edelberg, J. M., Fras, Z., Goodman, S. G., Halvorsen, S., Hanotin, C., et al. (2020). Effect of Alirocumab on Lipoprotein (a) and Cardiovascular Risk After Acute Coronary Syndrome. J Am Coll Cardiol 75, 133-144.
Blasius, A. L., and Beutler, B. (2010). Intracellular toll-like receptors. Immunity 32, 305-315.
Bosse, Y., Miqdad, A., Fournier, D., Pepin, A., Pibarot, P., and Mathieu, P. (2009). Refining molecular pathways leading to calcific aortic valve stenosis by studying gene expression profile of normal and calcified stenotic human aortic valves. Circ Cardiovasc Genet 2, 489-498.
Bouxsein, M. L., Boyd, S. K., Christiansen, B. A., Guldberg, R. E., Jepsen, K. J., and Muller, R. (2010). Guidelines for assessment of bone microstructure in rodents using micro-computed tomography. J Bone Miner Res 25, 1468-1486.
Bouxsein, M. L., Devlin, M. J., Glatt, V., Dhillon, H., Pierroz, D. D., and Ferrari, S. L. (2009). Mice lacking beta-adrenergic receptors have increased bone mass but are not protected from deleterious skeletal effects of ovariectomy. Endocrinology 150, 144-152.
Bouxsein, M. L., Myers, K. S., Shultz, K. L., Donahue, L. R., Rosen, C. J., and Beamer, W. G. (2005). Ovariectomy-induced bone loss varies among inbred strains of mice. J Bone Miner Res 20, 1085-1092.
Bunting, R. A., Duffy, K. E., Lamb, R. J., San Mateo, L. R., Smalley, K., Raymond, H., Liu, X., Petley, T., Fisher, J., Beck, H., et al. (2011). Novel antagonist antibody to TLR3 blocks poly(I:C)-induced inflammation in vivo and in vitro. Cell Immunol 267, 9-16.
Cavassani, K. A., Ishii, M., Wen, H., Schaller, M. A., Lincoln, P. M., Lukacs, N. W., Hogaboam, C. M., and Kunkel, S. L. (2008). TLR3 is an endogenous sensor of tissue necrosis during acute inflammatory events. J Exp Med 205, 2609-2621.
Chen, G. Y., and Nunez, G. (2010). Sterile inflammation: sensing and reacting to damage. Nat Rev Immunol 10, 826-837.
Chen, H. Y., Dufresne, L., Burr, H., Ambikkumar, A., Yasui, N., Luk, K., Ranatunga, D. K., Whitmer, R. A., Lathrop, M., Engert, J. C., et al. (2018). Association of LPA Variants With Aortic Stenosis: A Large-Scale Study Using Diagnostic and Procedural Codes From Electronic Health Records. JAMA Cardiol 3, 18-23.
Cheng, K., Wang, X., and Yin, H. (2011). Small-molecule inhibitors of the TLR3/dsRNA complex. J Am Chem Soc 133, 3764-3767.
Choe, J., Kelker, M. S., and Wilson, I. A. (2005). Crystal structure of human toll-like receptor 3 (TLR3) ectodomain. Science 309, 581-585.
Connell P S, Han R I, Grande-Allen K J (2012). Differentiating the aging of the mitral valve from human and canine myxomatous degeneration. J Vet Cardiol, 14(1), 31-45.
Consortium, G. T. (2013). The Genotype-Tissue Expression (GTEx) project. Nat Genet 45, 580-585.
Crow, Y. J. & Manel, N. Aicardi-Goutières syndrome and the type I interferonopathies. Nature Reviews Immunology 15, 429-440, doi:10.1038/nri3850 (2015).
Danielsen, R., Aspelund, T., Harris, T. B., and Gudnason, V. (2014). The prevalence of aortic stenosis in the elderly in Iceland and predictions for the coming decades: the AGES-Reykjavik study. Int J Cardiol 176, 916-922.
Derbali H, Bosse Y, Cote N, Pibarot P, Audet A, Pepin A, et al. (2010). Increased biglycan in aortic valve stenosis leads to the overexpression of phospholipid transfer protein via Toll-like receptor 2. Am J Pathol, 176(6), 2638-45.
Drolet, M. C., Roussel, E., Deshaies, Y., Couet, J. & Arsenault, M. A high fat/high carbohydrate diet induces aortic valve disease in C57BL/6J mice. J. Am. Coll. Cardiol. 47, 850-855, doi:10.1016/j.jacc.2005.09.049 (2006).
Ducy, P., Schinke, T. & Karsenty, G. The osteoblast: a sophisticated fibroblast under central surveillance. Science 289, 1501-1504, doi:10.1126/science.289.5484.1501 (2000).
Duque, G., Huang, D. C., Dion, N., Macoritto, M., Rivas, D., Li, W., Yang, X. F., Li, J., Lian, J., Marino, F. T., et al. (2011). Interferon-gamma plays a role in bone formation in vivo and rescues osteoporosis in ovariectomized mice. J Bone Miner Res 26, 1472-1483.
Emsley, P., Lohkamp, B., Scott, W. G., and Cowtan, K. (2010). Features and development of Coot. Acta Crystallogr D66, 486-501.
Eveborn, G. W., Schirmer, H., Heggelund, G., Lunde, P., and Rasmussen, K. (2013). The evolving epidemiology of valvular aortic stenosis. the Tromso study. Heart 99, 396-400.
Fernandez-Recio, J., Totrov, M., Skorodumov, C., and Abagyan, R. (2005). Optimal docking area: A new method for predicting protein-protein interaction sites. Proteins: Structure, Function, and Bioinformatics 58, 134-143.
Fleming, A., Sato, M., and Goldsmith, P. (2005). High-throughput in vivo screening for bone anabolic compounds with zebrafish. Journal of biomolecular screening 10, 823-831.
Franceschi, C., Garagnani, P., Parini, P., Giuliani, C., and Santoro, A. (2018). Inflammaging: a new immune-metabolic viewpoint for age-related diseases. Nat Rev Endocrinol 14, 576-590.
Frantz, S., Kobzik, L., Kim, Y. D., Fukazawa, R., Medzhitov, R., Lee, R. T., and Kelly, R. A. (1999). Toll4 (TLR4) expression in cardiac myocytes in normal and failing myocardium. J Clin Invest 104, 271-280.
Furukawa H, Chikada M, K. Yokoyama M, Arito M, S. Kurokawa M, Sato T, et al. (2016). Characterization of small leucine-rich proteoglycans in aortic valves of patients with aortic valve stenosis. Integrative Molecular Medicine, 3(5).
Gao, D. et al. TLR3 controls constitutive IFN-β antiviral immunity in human fibroblasts and cortical neurons. The Journal of Clinical Investigation 131, doi:10.1172/JCI134529 (2021).
Garcia-Cattaneo, A., Gobert, F. X., Muller, M., Toscano, F., Flores, M., Lescure, A., Del Nery, E., and Benaroch, P. (2012). Cleavage of Toll-like receptor 3 by cathepsins B and H is essential for signaling. Proc Natl Acad Sci USA 109, 9053-9058.
Garcia-Rodriguez, C., Parra-Izquierdo, I., Castanos-Mollor, I., Lopez, J., San Roman, J. A., and Sanchez Crespo, M. (2018). Toll-Like Receptors, Inflammation, and Calcific Aortic Valve Disease. Front Physiol 9, 201.
Gollmann-Tepeköylü, C., Lobenwein, D., Theurl, M., Primessnig, U., Lener, D., Kirchmair, E., Mathes, W., Graber, M., Pölzl, L., An, A., et al. (2018). Shock Wave Therapy Improves Cardiac Function in a Model of Chronic Ischemic Heart Failure: Evidence for a Mechanism Involving VEGF Signaling and the Extracellular Matrix. Journal of the American Heart Association 7.
Gotting C, Hendig D, Adam A, Schon S, Schulz V, Szliska C, et al. (2005). Elevated xylosyltransferase I activities in pseudoxanthoma elasticum (PXE) patients as a marker of stimulated proteoglycan biosynthesis. J Mol Med (Berl), 83(12), 984-92.
Grandeallen K, Osman N, Ballinger M, Dadlani H, Marasco S, Little P (2007). Glycosaminoglycan synthesis and structure as targets for the prevention of calcific aortic valve disease. Cardiovasc Res, 76(1), 19-28.
Groth, S. Fazekas de St, and Doris Scheidegger. “Production of monoclonal antibodies: strategy and tactics.” Journal of immunological methods 35.1-2 (1980): 1-21.
Hessle, L., Johnson, K. A., Anderson, H. C., Narisawa, S., Sali, A., Goding, J. W., Terkeltaub, R., and Millan, J. L. (2002). Tissue-nonspecific alkaline phosphatase and plasma cell membrane glycoprotein-1 are central antagonistic regulators of bone mineralization. Proc Natl Acad Sci USA 99, 9445-9449.
Hildebrand, T., and Rüegsegger, P. (1997). A new method for the model-independent assessment of thickness in three-dimensional images. J Microsc 185, 67-75.
Holfeld, J., Tepekoylu, C., Reissig, C., Lobenwein, D., Scheller, B., Kirchmair, E., Kozaryn, R., Albrecht-Schgoer, K., Krapf, C., Zins, K., et al. (2016). Toll-like receptor 3 signalling mediates angiogenic response upon shock wave treatment of ischaemic muscle. Cardiovasc Res 109, 331-343.
Huang da, W., Sherman, B. T., and Lempicki, R. A. (2009). Systematic and integrative analysis of large gene lists using DAVID bioinformatics resources. Nat Protoc 4, 44-57.
Hutcheson, J. D. et al. Cadherin-11 regulates cell-cell tension necessary for calcific nodule formation by valvular myofibroblasts. Arterioscler. Thromb. Vasc. Biol. 33, 114-120, doi:10.1161/atvbaha.112.300278 (2013).
Jin C L, Oh J H, Han M, Shin M K, Yao C, Park C H, et al. (2015). UV irradiation-induced production of monoglycosylated biglycan through downregulation of xylosyltransferase 1 in cultured human dermal fibroblasts. J Dermatol Sci, 79(1), 20-9.
Kamburov, A., Pentchev, K., Galicka, H., Wierling, C., Lehrach, H., and Herwig, R. (2011). ConsensusPathDB: toward a more complete picture of cell biology. Nucleic Acids Res 39, D712-717.
Kawai, T., and Akira, S. (2010). The role of pattern-recognition receptors in innate immunity: update on Toll-like receptors. Nat Immunol 11, 373-384.
Kiechl, S., Lorenz, E., Reindl, M., Wiedermann, C. J., Oberhollenzer, F., Bonora, E., Willeit, J., and Schwartz, D. A. (2002). Toll-like receptor 4 polymorphisms and atherogenesis. N Engl J Med 347, 185-192.
Kim, J. H., Song, D. H., Youn, S. J., Kim, J. W., Cho, G., Kim, S. C., Lee, H., Jin, M. S., and Lee, J. O. (2016). Crystal structures of mono- and bi-specific diabodies and reduction of their structural flexibility by introduction of disulfide bridges at the Fv interface. Sci Rep 6, 34515.
Krissinel, E. (2010). Crystal contacts as nature's docking solutions. Journal of Computational Chemistry 31, 133-143.
Krissinel, E., and Henrick, K. (2007). Inference of Macromolecular Assemblies from Crystalline State. J Mol Biol 372, 774-797.
Lehmann S, Walther T, Kempfert J, Rastan A, Garbade J, Dhein S, et al. (2009). Mechanical strain and the aortic valve: influence on fibroblasts, extracellular matrix, and potential stenosis. Ann Thorac Surg, 88(5), 1476-83.
Leonard, J. N., Ghirlando, R., Askins, J., Bell, J. K., Margulies, D. H., Davies, D. R., and Segal, D. M. (2008). The TLR3 signaling complex forms by cooperative receptor dimerization. Proc Natl Acad Sci USA 105, 258-263.
Li, J. (2013). JAK-STAT and bone metabolism. JAKSTAT 2, e23930.
Liu, A. C., Joag, V. R., and Gotlieb, A. I. (2007). The emerging role of valve interstitial cell phenotypes in regulating heart valve pathobiology. Am J Pathol 171, 1407-1418.
Liu, L., Botos, I., Wang, Y., Leonard, J. N., Shiloach, J., Segal, D. M., and Davies, D. R. (2008). Structural basis of toll-like receptor 3 signaling with double-stranded RNA. Science 320, 379-381.
Lobenwein, D., Tepekoylu, C., Kozaryn, R., Pechriggl, E. J., Bitsche, M., Graber, M., Fritsch, H., Semsroth, S., Stefanova, N., Paulus, P., et al. (2015). Shock Wave Treatment Protects From Neuronal Degeneration via a Toll-Like Receptor 3 Dependent Mechanism: Implications of a First-Ever Causal Treatment for Ischemic Spinal Cord Injury. Journal of the American Heart Association 4, e002440.
Lyskov, S., and Gray, J. J. (2008). The RosettaDock server for local protein-protein docking. Nucleic Acids Research 36, W233-W238.
Martin-Rojas T, Mourino-Alvarez L, Alonso-Orgaz S, Rosello-Lleti E, Calvo E, Lopez-Almodovar L F, et al. (2015). iTRAQ proteomic analysis of extracellular matrix remodeling in aortic valve disease. Sci Rep, 5(1).
Meng, X., A, L., Song, Y., Babu, A., Yang, X., Wang, M., Weyant, M. J., Dinarello, C. A., Cleveland, J. C., Jr., and Fullerton, D. A. (2008). Expression of functional Toll-like receptors 2 and 4 in human aortic valve interstitial cells: potential roles in aortic valve inflammation and stenosis. Am J Physiol Cell Physiol 294, C29-35.
Mis, E. K., Liem, K. F., Kong, Y., Schwartz, N. B., Domowicz, M., and Weatherbee, S. D. (2014). Forward genetics defines Xylt1 as a key, conserved regulator of early chondrocyte maturation and skeletal length. Dev Biol 385, 67-82.
Moreth, K., Brodbeck, R., Babelova, A., Gretz, N., Spieker, T., Zeng-Brouwers, J., Pfeilschifter, J., Young, M. F., Schaefer, R. M., and Schaefer, L. (2010). The proteoglycan biglycan regulates expression of the B cell chemoattractant CXCL13 and aggravates murine lupus nephritis. J Clin Invest 120, 4251-4272.
Muller B, Prante C, Gastens M, Kuhn J, Kleesiek K, Gotting C (2008). Increased levels of xylosyltransferase I correlate with the mineralization of the extracellular matrix during osteogenic differentiation of mesenchymal stem cells. Matrix Biol, 27(2), 139-49.
Munns C F, Fahiminiya S, Poudel N, Munteanu M C, Majewski J, Sillence D O, et al. (2015). Homozygosity for frameshift mutations in XYLT2 result in a spondylo-ocular syndrome with bone fragility, cataracts, and hearing defects. Am J Hum Genet, 96(6), 971-8.
Nakashima Y, Wight T N, Sueishi K (2008). Early atherosclerosis in humans: role of diffuse intimal thickening and extracellular matrix proteoglycans. Cardiovasc Res, 79(1), 14-23.
Naschberger, A., Fürnrohr, B., Lenac Rovis, T., Malic, S., Scheffzek, K., Dieplinger, H., and Rupp, B. (2016). The N14 anti-afamin antibody Fab: a rare VL1 CDR glycosylation, crystallographic re-sequencing, molecular plasticity, and conservative versus enthusiastic modelling. Acta Crystallogr D72, 1267-1280.
Neufeld E B, Zadrozny L M, Phillips D, Aponte A, Yu Z X, Balaban R S (2014). Decorin and biglycan retain LDL in disease-prone valvular and aortic subendothelial intimal matrix. Atherosclerosis, 233(1), 113-21.
Pierce, B. G., Wiehe, K., Hwang, H., Kim, B.-H., Vreven, T., and Weng, Z. (2014). ZDOCK server: interactive docking prediction of protein-protein complexes and symmetric multimers. Bioinformatics 30, 1771-1773.
Pohar J, Pirher N, Bencina M, Mancek-Keber M and Jerala R (2014). The Ectodomain of TLR3 Receptor Is Required for Its Plasma Membrane Translocation. PLOS ONE 9(3): e92391.
Pryshchep, O., Ma-Krupa, W., Younge, B. R., Goronzy, J. J., and Weyand, C. M. (2008). Vessel-specific Toll-like receptor profiles in human medium and large arteries. Circulation 118, 1276-1284.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47.
Robinson, M. D., McCarthy, D. J., and Smyth, G. K. (2010). edgeR: a Bioconductor package for differential expression analysis of digital gene expression data. Bioinformatics 26, 139-140.
Roedig H, Nastase M V, Wygrecka M, Schaefer L. (2019). Breaking down chronic inflammatory diseases: the role of biglycan in promoting a switch between inflammation and autophagy. FEBS J. 286(15), 2965-79.
Rossebo, A. B., Pedersen, T. R., Boman, K., Brudi, P., Chambers, J. B., Egstrup, K., Gerdts, E., Gohlke-Barwolf, C., Holme, I., Kesaniemi, Y. A., et al. (2008). Intensive lipid lowering with simvastatin and ezetimibe in aortic stenosis. N Engl J Med 359, 1343-1356.
Schon S, Schulz V, Prante C, Hendig D, Szliska C, Kuhn J, et al. (2006). Polymorphisms in the xylosyltransferase genes cause higher serum XT-I activity in patients with pseudoxanthoma elasticum (PXE) and are involved in a severe disease course. J Med Genet, 43(9), 745-9.
Schreml J, Durmaz B, Cogulu O, Keupp K, Beleggia F, Pohl E, et al. (2014). The missing “link”: an autosomal recessive short stature syndrome caused by a hypofunctional XYLT1 mutation. Hum Genet, 133(1), 29-39.
Scott, P. G., Dodd, C. M., Bergmann, E. M., Sheehan, J. K., and Bishop, P. N. (2006). Crystal structure of the biglycan dimer and evidence that dimerization is essential for folding and stability of class I small leucine-rich repeat proteoglycans. J Biol Chem 281, 13324-13332.
Silkoff, P. E., Flavin, S., Gordon, R., Loza, M. J., Sterk, P. J., Lutter, R., Diamant, Z., Turner, R. B., Lipworth, B. J., Proud, D., et al. (2018). Toll-like receptor 3 blockade in rhinovirus-induced experimental asthma exacerbations: A randomized controlled study. J Allergy Clin Immunol 141, 1220-1230.
Stephens E H, Saltarrelli J G, Baggett L S, Nandi I, Kuo J J, Davis A R, et al. (2011). Differential proteoglycan and hyaluronan distribution in calcified aortic valves. Cardiovasc Pathol, 20(6), 334-42.
Sturn, A., Quackenbush, J., and Trajanoski, Z. (2002). Genesis: cluster analysis of microarray data. Bioinformatics 18, 207-208.
Song R, Fullerton D A, A L, Zheng D, Zhao K-s, Meng X. (2014). BMP-2 and TGF-β1 mediate biglycan-induced pro-osteogenic reprogramming in aortic valve interstitial cells. J Mol Med, 93(4), 403-12.
Song R, Zeng Q, A L, Yu J A, Cleveland J C, Zhao K S, et al. (2012). Biglycan induces the expression of osteogenic factors in human aortic valve interstitial cells via Toll-like receptor-2. Arterioscler Thromb Vasc Biol, 32(11), 2711-20.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Sudlow, C., Gallacher, J., Allen, N., Beral, V., Burton, P., Danesh, J., Downey, P., Elliott, P., Green, J., Landray, M., et al. (2015). UK biobank: an open access resource for identifying the causes of a wide range of complex diseases of middle and old age. PLOS Med 12, e1001779.
Tadros, T. M., Klein, M. D., and Shapira, O. M. (2009). Ascending aortic dilatation associated with bicuspid aortic valve: pathophysiology, molecular biology, and clinical implications. Circulation 119, 880-890.
Takayanagi, H., Kim, S., Matsuo, K., Suzuki, H., Suzuki, T., Sato, K., Yokochi, T., Oda, H., Nakamura, K., Ida, N., et al. (2002). RANKL maintains bone homeostasis through c-Fos-dependent induction of interferon-beta. Nature 416, 744-749.
Takemura, N., Kawasaki, T., Kunisawa, J., Sato, S., Lamichhane, A., Kobiyama, K., Aoshi, T., Ito, J., Mizuguchi, K., Karuppuchamy, T., et al. (2014). Blockade of TLR3 protects mice from lethal radiation-induced gastrointestinal syndrome. Nature communications 5, 3492.
Tepekoylu, C., Lobenwein, D., Urbschat, A., Graber, M., Pechriggl, E. J., Fritsch, H., Paulus, P., Grimm, M., and Holfeld, J. (2018). Shock wave treatment after hindlimb ischaemia results in increased perfusion and M2 macrophage presence. J Tissue Eng Regen Med 12, e486-e494.
Thanassoulis, G., Campbell, C. Y., Owens, D. S., Smith, J. G., Smith, A. V., Peloso, G. M., Kerr, K. F., Pechlivanis, S., Budoff, M. J., Harris, T. B., et al. (2013). Genetic associations with valvular calcification and aortic stenosis. N Engl J Med 368, 503-512.
Tufvesson, E., Malmstrom, J., Marko-Varga, G., and Westergren-Thorsson, G. (2002). Biglycan isoforms with differences in polysaccharide substitution and core protein in human lung fibroblasts. Eur J Biochem 269, 3688-3696.
Wagner, G. P., and Zhang, J. (2011). The pleiotropic structure of the genotype-phenotype map: the evolvability of complex organisms. Nat Rev Genet 12, 204-213.
Walker, M. B., and Kimmel, C. B. (2007). A two-color acid-free cartilage and bone stain for zebrafish larvae. Biotech Histochem 82, 23-28.
Wang, Y., Liu, L., Davies, D. R., and Segal, D. M. (2010). Dimerization of Toll-like receptor 3 (TLR3) is required for ligand binding. J Biol Chem 285, 36836-36841.
Weiss, R. M., Ohashi, M., Miller, J. D., Young, S. G., and Heistad, D. D. (2006). Calcific aortic valve stenosis in old hypercholesterolemic mice. Circulation 114, 2065-2069.
Yang, X., Fullerton, D. A., Su, X., A, L., Cleveland, J. C., Jr., and Meng, X. (2009). Pro-osteogenic phenotype of human aortic valve interstitial cells is associated with higher levels of Toll-like receptors 2 and 4 and enhanced expression of bone morphogenetic protein 2. J Am Coll Cardiol 53, 491-500.
Yauy K, Tran Mau-Them F, Willems M, Coubes C, Blanchet P, Herlin C, et al. (2018). B3GAT3-related disorder with craniosynostosis and bone fragility due to a unique mutation. Genet Med, 20(2), 269-74.
Yavorska, O. O., and Burgess, S. (2017). MendelianRandomization: an R package for performing Mendelian randomization analyses using summarized data. Int J Epidemiol 46, 1734-1739.
Zeng, Q., Song, R., A, L., Weyant, M. J., Lee, J., Xu, D., Fullerton, D. A., and Meng, X. (2013). Notch1 promotes the pro-osteogenic response of human aortic valve interstitial cells via modulation of ERK1/2 and nuclear factor-kappaB activation. Arterioscler Thromb Vasc Biol 33, 1580-1590.
Zhan, Q., Song, R., Zeng, Q., Yao, Q., A, L., Xu, D., Fullerton, D. A., and Meng, X. (2015). Activation of TLR3 induces osteogenic responses in human aortic valve interstitial cells through the NF-kappaB and ERK1/2 pathways. Int J Biol Sci 11, 482-493.
Zhang, S. Y. et al. TLR3 deficiency in patients with herpes simplex encephalitis. Science 317, 1522-1527, doi:10.1126/science.1139522 (2007).
Zhang, S. Y. et al. Inborn errors of interferon (IFN)-mediated immunity in humans: insights into the respective roles of IFN-alpha/beta, IFN-gamma, and IFN-lambda in host defense. Immunol. Rev. 226, 29-40, doi:10.1111/j.1600-065X.2008.00698.x (2008).
Zhang, Q. et al. Inborn errors of type I IFN immunity in patients with life-threatening COVID-19. Science 370, eabd4570, doi:10.1126/science.abd4570 (2020).
Zhao, W., Qi, J., Wang, L., Zhang, M., Wang, P., and Gao, C. (2012). LY294002 inhibits TLR3/4-mediated IFN-beta production via inhibition of IRF3 activation with a PI3K-independent mechanism. FEBS Lett 586, 705-710.
Zheng, K. H., Tsimikas, S., Pawade, T., Kroon, J., Jenkins, W. S. A., Doris, M. K., White, A. C., Timmers, N., Hjortnaes, J., Rogers, M. A., et al. (2019). Lipoprotein (a) and Oxidized Phospholipids Promote Valve Calcification in Patients With Aortic Stenosis. J Am Coll Cardiol 73, 2150-2162.
Zhu J, Smith K., Hsieh P N, Mburu Y K, Chattopadhy S, Sen G C, Sarkar S N, (2010). A high throughput screening for TLR3-IRF3 signaling pathway modulators identifies several antipsychotic drugs as TLR inhibitors. J Immunol. 184, 5768-5776.
Rutsch, F. et al. A Specific IFIH1 Gain-of-Function Mutation Causes Singleton-Merten Syndrome. The American Journal of Human Genetics 96, 275-282, doi:10.1016/j.ajhg.2014.12.014 (2015).

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. An inhibitor of toll-like receptor 3 (TLR3) for use in the treatment of a disease associated with cardiovascular calcification, preferably, wherein the disease is in an early stage.

2. A method of treating a disease associated with cardiovascular calcification, the method comprising administering an inhibitor of toll-like receptor 3 (TLR3), preferably, wherein the disease is in an early stage.

3. The inhibitor for use of claim 1, or the method of claim 2, wherein the disease is associated with calcification of the aortic valve and/or calcification of the coronary arteries and/or calcification of the great arteries,
preferably, wherein the disease is associated with calcification of the aortic valve.

4. The inhibitor for use of claim 1 or 3, or the method of claim 2 or 3, wherein the disease is calcific aortic valve disease (CAVD) and/or coronary heart disease (CHD),
preferably, wherein the disease is CAVD,
optionally, wherein the disease is early stage CAVD and/or early stage CHD.

5. The inhibitor for use of claim 4, or the method of claim 4, wherein the CAVD manifests in aortic sclerosis and/or aortic stenosis.

6. The inhibitor for use of any one of claims 1, 3 to 5, or the method of any one of claims 2 to 5, wherein the cardiovascular calcification is induced by mechanical stress, irradiation, chronic kidney disease, factors promoting atherosclerosis, genetic predisposition or a combination thereof,
preferably, wherein the cardiovascular calcification is induced by a combination of mechanical stress or factors promoting atherosclerosis,
further preferably, wherein the cardiovascular calcification is induced by mechanical stress,
further preferably, wherein the mechanical stress is hemodynamic stress.

7. The inhibitor for use of any one of claims 1, 3 to 6, or the method of any one of claims 2 to 6, wherein the inhibitor is one or more of (R)-2-(3-Chloro-6-fluorobenzo [b] thiophene-2-carboxamido)-3-phenyl-propanoic acid], Levocetirizine, ORF 1329L, Sertraline, Fluphenazine, ZL0420, ZL0454, Quinacrine, Chloroquine and Amiodarone.

8. A method for assessing whether a subject is at increased risk of developing a disease associated with cardiovascular calcification, said method comprising,
(i) determining in a sample from said subject whether one or more genomic sequences in or near one or more gene comprises one or more genetic variant; and
(ii) assessing that said subject is at increased risk of developing a disease associated with cardiovascular calcification when the one or more genetic variant in the one or more genomic sequences in or near said one or more gene is present,
preferably, wherein the disease is at an early stage.

9. The method of claim 8, wherein the one or more gene is one or more of JAK1, TLR3, IFNB1, IFNA1, XYLT1 or IFNAR1.

10. The method of claim 8 or 9, wherein the risk of developing a disease associated with cardiovascular calcification is increased at least 1.5-fold.

11. The method of any one of claims 8 to 10, wherein the genetic variant is a single-nucleotide polymorphism (SNP).

12. The method of any one of items 8 to 11, wherein the genetic variant is one or more of
(a) a genomic sequence in or near the human JAK1 gene comprising one or more of:
(i) variant rs143732508 at position 1:65335640 comprising the nucleobase G;
(ii) variant rs564691204 at position 1:65342993 comprising the nucleobase T;
(iii) variant rs528952911 at position 1:65347527 comprising the nucleobase C; and/or
(iv) variant rs146653955 at position 1:65380580 comprising the nucleobase C;
(b) a genomic sequence in or near the human TLR3 gene comprising one or more of:
(i) variant rs548870644 at position 4:186953463 comprising the nucleobase G; and/or
(ii) variant rs184106700 at position 4:187028029 comprising the nucleobase G;
(c) a genomic sequence in or near the human IFNB1 gene and comprises one or more of:
(i) variant rs569915578 at position 9:21119979 comprising the nucleobase T;
(ii) variant rs755535058 at position 9:21120058 comprising the nucleobase T; and/or
(d) a genomic sequence in or near the human IFNA1 gene comprising the variant rs551992948 at position 9:21457591 comprising the nucleobase C;
(e) a genomic sequence in or near the human XYLT1 gene comprising one or more of:
(i) variant rs118001479 at position 16:17153381 comprising the nucleobase A;
(ii) variant rs550834189 at position 16:17283730 comprising the nucleobase A;
(iii) variant rs531295111 at position 16:17289368 comprising the nucleobase C;
(iv) variant rs62033189 at position 16:17342509 comprising the nucleobase C;
(v) variant rs34588333 at position 16:17345488 comprising the nucleobase A; and/or
(vi) variant rs936346 at position 16:17376126 comprising the nucleobase C;
(f) a genomic sequence in or near the human IFNAR1 gene comprising the variant rs554831417 at position 21:34683984 comprising the nucleobase T.

13. The method of any one of claims 8 to 12, wherein the disease is associated with calcification of the aortic valve and/or calcification of the coronary arteries and/or calcification of the great arteries
preferably, wherein the disease is associated with calcification of the aortic valve.

14. The method of any one of claims 8 to 13, wherein the disease is calcific aortic valve disease (CAVD) and/or coronary heart disease (CHD),
preferably, wherein the disease is CAVD,
optionally, wherein the disease is early stage CAVD and/or early stage CHD.

15. The method of any one of claims 8 to 14, wherein the cardiovascular calcification is induced by mechanical stress, irradiation, chronic kidney disease, factors promoting atherosclerosis or a combination thereof
preferably, wherein the cardiovascular calcification is induced by a combination of mechanical stress and factors promoting atherosclerosis,
further preferably, wherein the cardiovascular calcification is induced by mechanical stress,
further preferably, wherein the mechanical stress is hemodynamic stress.

16. Use of a binding molecule, a nucleic acid, a nucleic acid probe, a primer, a primer pair, a biotinylated primer, a SNP microarray, a single-stranded oligonucleotide probe specific for one or more genetic variant of the one or more genomic sequences in or near a gene as defined in any one of claims 8 to 15 for assessing whether a subject is at increased risk of developing a disease associated with cardiovascular calcification.

17. A drug for use in the treatment of a subject assessed to be at increased risk of developing a disease associated with cardiovascular calcification according to any one of claims 8 to 16.

18. The drug of claim 17, wherein the drug comprises an inhibitor of toll-like receptor 3 (TLR3).

19. A substance for use in a method of in vivo diagnosis of a disease associated with cardiovascular calcification in a subject, the method comprising:
(i) administering the substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification,
preferably, wherein the disease is at an early stage.

20. A method for diagnosing in vivo a disease associated with cardiovascular calcification in a subject, the method comprising,
(i) administering a substance to said subject, and
(ii) detecting if said substance accumulates in cardiac tissue,
wherein the level of accumulation of said substance in the cardiac tissue is indicative for said subject being at increased risk of developing or suffering from a disease associated with cardiovascular calcification,
preferably, wherein the disease is at an early stage.

21. The substance for use of claim 19, or the method of claim 20, wherein the substance is radioactively labelled and binds TLR3,
preferably, wherein the substance comprises a TLR3 specific ligand, antibody or nucleic acid probe.

22. The substance for use of claim 19 or 21, or the method of claim 20 or 21, wherein the substance is administered to the subject intravenously and an accumulation of said substance is detected by positron emission tomography (PET).

23. The substance for use of any one of claims 19 to 22, or the method of claims 20 to 22, wherein the cardiac tissue is the aortic valve and the accumulation is indicative for the subject being at increased risk of developing or suffering from CAVD, and/or
wherein the cardiac tissue is the coronary arteries and the accumulation is indicative for the subject being at increased risk of developing or suffering from CHD, and/or
wherein the cardiac tissue is the great arteries and the accumulation is indicative for the subject being at increased risk of developing or suffering from CHD,
preferably, wherein the cardiac tissue is the aortic valve and the accumulation is indicative for the subject being at increased risk of developing or suffering from CAVD.

24. A kit comprising the binding molecule, the nucleic acid, the nucleic acid probe, the primer, the primer pair, the biotinylated primer, the SNP microarray, the single-stranded oligonucleotide probe of claim 16 or the substance of any one of claims 19 to 23.

25. Use of the kit as defined in claim 24 for carrying out the method or use as defined in any one of claims 8 to 16 and 19 to 23.

26. The substance of any one of claims 19 to 23.

27. The substance of any one of claims 19 to 23 for use in in vivo diagnostics.


SEQUENCE LISTING (9 sequences)

SEQ ID NO: 1 - DNA, 21 bp, Homo sapiens
  source 1..21: mol_type = other DNA
aggaaaggctagcagtcatcc

SEQ ID NO: 2 - DNA, 20 bp, Homo sapiens
  source 1..20: mol_type = other DNA
taacagtgcacttggtggtg

SEQ ID NO: 3 - DNA, 22 bp, Homo sapiens
  source 1..22: mol_type = other DNA
tgagaacctcctggctaatgtc

SEQ ID NO: 4 - DNA, 20 bp, Homo sapiens
  source 1..20: mol_type = other DNA
ttttcaggtgcagactgctc

SEQ ID NO: 5 - DNA, 21 bp, Homo sapiens
  source 1..21: mol_type = other DNA
gctgctgatgcctgagaaggt

SEQ ID NO: 6 - DNA, 21 bp, Homo sapiens
  source 1..21: mol_type = other DNA
gacaaaggcgattctgaccgg

SEQ ID NO: 7 - DNA, 20 bp, Mus musculus
  source 1..20: mol_type = other DNA
ttgtcttctgcacgaacctg

SEQ ID NO: 8 - DNA, 21 bp, Mus musculus
  source 1..21: mol_type = other DNA
ccgttcccaactttgtagatg

SEQ ID NO: 9 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH